(19) United States
(12) Patent Application Publication
(10) Pub. No.: US 2008/0021217 A1
(43) Pub. Date: Jan. 24, 2008

(54) HETEROCYCLIC INHIBITORS OF RHO KINASE

(75) Inventors: Allen J. Borchardt, San Diego, CA (US); Mehmet Kahraman, San Diego, CA (US); Travis G. Cook, La Jolla, CA (US); Robert L. Davis, Carlsbad, CA (US); Elizabeth M. M. Gardiner, San Diego, CA (US); James W. Malecha, San Diego, CA (US); Stewart A. Noble, San Diego, CA (US); Thomas J. Prins, San Diego, CA (US); Michael Sertic, San Diego, CA (US); Dana L. Siegel, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignees: Allen J. Borchardt, San Diego, CA (US); Mehmet Kahraman, San Diego, CA (US); Travis G. Cook, La Jolla, CA (US); Robert L. Davis, Carlsbad, CA (US); Elizabeth M. M. Gardiner, San Diego, CA (US); James W. Malecha, San Diego, CA (US); Stewart A. Noble, San Diego, CA (US); Thomas J. Prins, San Diego, CA (US); Michael Sertic, Euclid, OH (US); Dana L. Siegel, San Diego, CA (US)

(21) Appl. No.: 11/780,735

(22) Filed: Jul. 20, 2007

Related U.S. Application Data
(60) Provisional application No. 60/832,634, filed on Jul. 20, 2006.
(60) Provisional application No. 60/915,772, filed on May 3, 2007.

(51) Int. Cl. A61K 31/44; A61K 31/495; A61K 31/519; C07D 237/26; C07D 401/14; C07D 413/00; C07D 413/14; C07D 417/04; C07D 487/00

(52) U.S. Cl. 544/235; 514/249; 514/258.1; 514/299; 514/341; 514/342; 544/236; 546/112; 546/269.4; 546/270.4; 546/271.4; 546/272.4; 546/274.1; 546/275.4; 546/275.7

(57) ABSTRACT

The present invention relates to compounds and methods which may be useful as inhibitors of Rho kinase for the treatment or prevention of disease.

This application claims the benefit of priority of U.S. provisional application Ser. No. 60/832,634, filed Jul. 20, 2006 and U.S. provisional application Ser. No. 60/915,772, filed May 3, 2007, the disclosures of which is hereby incorporated by reference as if written herein in its entirety.

The present invention is directed to new heteroaryl compounds and compositions and their application as pharmaceuticals for the treatment of disease. Methods of inhibition of Rho kinase activity in a human or animal subject are also provided for the treatment of diseases such as ophthalmologic diseases.

Many cell signaling events activate one or more members of the small monomeric GTPase superfamily. The Rho subfamily of GTPases (consisting of RhoA, RhoB, and RhoC) transmits signals, frequently from cell surface receptors, to effectors that play critical roles in control of cytoskeletal dynamics and gene regulation [Ridley, A. J., 2001, Trends Cell Biol. 11:471-477; Jaffe, A. B. and Hall, A., 2005, Annu Rev Cell Dev Biol. 21:247-269]. In particular, Rho-mediated effects on the cytoskeleton influence non-muscle cell shape, smooth muscle cell contraction, cell-cell and cell-matrix adhesion, intracellular vesicle transport, axonal and dendrite growth, vascular architecture, immune and inflammatory cell migration, and cleavage furrow formation and function during cell division [Bussey, H., 1996, Science. 272:224-225; Fukata, Y. et al., 2001, Trends Pharmacol Sci. 22:32-39; Luo, L., 2000, Nat Rev Neurosci. 1:173-180; Hu, E. and Lee, D., 2003, Curr Opin Investig Drugs. 4:1065-1075; Bokoch, G. M. 2005, Trends Cell Biol. 15:163-171; Wadsworth, P., 2005, Curr Biol. 15:R871-874].

Although the Rho GTPase cycle is complex, it can be briefly summarized as follows. Inactive, GDP-bound Rho, complexed with a GDP dissociation inhibitor protein (GDI), is recruited to the plasma membrane in response to signaling events, such as ligand binding to cell surface receptors. The GDI is displaced, whereby the inactive GDP-bound Rho is converted to active GTP-bound Rho by membrane-localized guanine-nucleotide exchange factors. GTP-bound Rho then binds and activates a number of effectors at the plasma membrane. Many proteins controlled by Rho activity have been identified, including a variety of protein and lipid kinases [Kaibuchi, K. et al., 1999, Annu Rev Biochem. 68:459-486; Bishop, A. L. and Hall, A., 2000, Biochem J. 348:241-255]. The intrinsic GTPase activity of Rho, stimulated by GTPase activating proteins, converts Rho back to the inactive, GDP-bound form, whereupon GDP-bound Rho can be extracted from the plasma membrane by the GDI (although in some instances, the GDI may extract GTP-bound Rho to extinguish a signal, or redirect GTP-bound Rho to a different compartment) [Sasaki T., and Takai Y., 1998, Biochem Biophys Res Commun. 245:641-645; Olofsson, B., 1999, Cell Signal. 11:545-554; Schmidt, A. and Hall, A., 2002, Genes Dev. 16:1587-1609; Moon, S. Y. and Zheng, Y., 2003, Trends Cell Biol. 13:13-22].

Of identified Rho effectors, the Rho-associated coiled-coil containing kinases, here referred to as Rho kinases, have been the subject of intense investigation in molecular and cell biological studies, and as pharmaceutical targets in multiple therapeutic areas. Rho kinases are serine-threonine protein kinases of approximately 160 kD molecular weight that contain an amino-terminal kinase catalytic domain, a long amphipathic alpha helical (coiled-coil) domain, an activated Rho binding domain, and a carboxy-terminal pleckstrin-homology domain (promoting binding to plasma membrane phosphoinositides) that is split by a cysteine rich zinc-finger like motif [Ishizaki, T., et al., 1996, EMBO J. 15, 1885-1893; Fujisawa, K. et al., 1996, J Biol Chem. 271:23022-23028; Matsui, T. et al., 1996, EMBO J. 15:2208-2216]. There are two known isoforms of Rho kinase, although splice variants may exist. These isoforms are referred to as Rho kinase (ROK) alpha (referred to here as ROCK2), and Rho kinase (ROK) beta, also known as p160 ROCK (referred to here as ROCK1) [Leung, T. et al., 1996, Mol Cell Biol. 16:5313-5327; Nakagawa, O. et al., 1996, FEBS Lett. 392:189-193]. Many protein kinases are controlled by reversible phosphorylation events that switch them between active and inactive states. By contrast, Rho kinases switch from low, basal activity to high activity by reversible binding to GTP-bound Rho. Active Rho kinases then phosphorylate additional effectors of Rho signaling in the vicinity of the plasma membrane. Both Rho kinases are expressed in a mostly ubiquitous fashion in mammalian tissues at low to moderate levels, although expression is highly enriched in some cell types. Rho kinases share functional homology in their catalytic domains with the protein kinase A and C families, and a variety of small molecule inhibitors of Rho kinases also bind and inhibit protein kinase A in particular [Breitenlechner, C. et al., 2003, Structure. 11:1595-1607]. ROCK1 has 64% sequence identity to ROCK2 throughout the protein structure, and the kinase domains are highly conserved (90% identical).

As effectors of Rho signaling, Rho kinases are directly involved in controlling cytoskeleton dynamics, gene regulation, cell proliferation, cell division, and cell survival. Constitutively active mutants of Rho kinases can be generated by truncating carboxy-terminal regions, as far as the kinase domain, suggesting important negative regulation by the carboxy-terminal sequences. Expressed in cells, these mutants generate phenotypes consistent with hyperactive Rho kinase activity (e.g. increased stress fiber formation and cell-substrate focal adhesions). By contrast, deletion of the catalytic domain of Rho kinases results in a trans-dominant inhibitory effect in cells [Amano, M. et al., 1997, Science. 275:1308-1311; Leung, T. et al., 1996, Mol Cell Biol. 16:5313-5327; Amano, M. et al., 1999, J Biol Chem. 274:32418-32424]. There is data consistent with separable functions for ROCK1 and ROCK2 in cells, although these observations may be cell-type specific [Yoneda, A. et al., 2005, J Cell Biol. 170:443-453]. Although genetic knockout of ROCK1 leads to perinatal lethality due to omphaloceles in newborns, and genetic knockout of ROCK2 leads to a high incidence of embryonic lethality due to poor placental development, neither knockout alone is consistent with the necessity of ROCK1 or ROCK2 for most normal cell behaviors of the embryo during development [Shimizu, Y. et al., 2005, J Cell Biol. 168:941-953; Thumkeo, D. et al., 2003, Mol Cell Biol. 23:5043-5055].

Rho kinases can phosphorylate a variety of substrates to control various aspects of cytoskeletal behavior [Riento, K. and Ridley, A. J. 2003, Nat Rev Mol Cell Biol. 4:446-456]. Many studies have focused on control of the myosin light chain (MLC) regulatory subunit. Phosphorylation of the MLC regulatory subunit leads to increased actomyosin activity (e.g. smooth muscle cell contraction or increased non-muscle cell stress fibers). Rho kinases stimulate actomyosin activity by direct phosphorylation of the MLC regulatory subunit, and by inactivation of myosin light chain phosphatase through the phosphorylation of its myosin binding subunit [Amano, M. et al., 1996, J Biol Chem. 271:20246-20249; Kimura, K. et al., 1996, Science. 273:245-248; Kureishi, Y. et al., 1997, J Biol Chem. 272:12257-12260]. LIM kinase, ezrin/radixin/moesin (ERM) family proteins, and adducin are some additional substrates of Rho kinases, and the phosphorylation of these and other proteins alters various aspects of cytoskeletal function [Oshiro, N., et al., 1998, J Biol Chem. 273:34663-34666; Kimura, K., et al., 1998, J Biol Chem. 273:5542-5548; Matsui, T., et al., 1998, J Cell Biol 140:647-657; Fukata, Y., et al., 1999, J Cell Biol. 145:347-361; Kosako, H., et al., 1997, J Biol Chem. 272:10333-10336; Goto, H., et al., 1998, J Biol Chem. 273:11728-11736; Maekawa, M., et al., 1999, Science. 285:895-898; Ohashi, K., et al., 2000, J Biol Chem. 275:3577-3582].

Small molecule compounds such as Y-27632, Y-32885, Y-39983, HA-1077 (fasudil), hydroxy-fasudil, and a dimethylated analog of fasudil (H-1152P, or HMN-1152) have been demonstrated to directly inhibit Rho kinases. The Y compounds, which are more selective Rho kinase inhibitors, contain a common pyridine moiety, while fasudil and its analogs contain a common isoquinoline scaffold. Crystal structures for the kinase domain of ROCK1 complexed with Y-27632, fasudil, hydroxy-fasudil, and H-1152P have been reported (Jacobs, M. et al., 2006, J Biol Chem. 281:260-268]. All of these compounds occupy part of the ATP-binding pocket, consistent with the fact that they are reversible ATP competitive inhibitors.

These same Rho kinase inhibitors are cell permeable, and cause changes in cytoskeletal function and cell behavior consistent with loss of Rho kinase activity, similar to effects of the trans-dominant inhibitory mutants. Effects have been observed both in cultured cells in vitro and in physiologically responsive tissues in vivo [Nagumo, H. et al., 2000, Am J Physiol Cell Physiol. 278:C57-C65; Sinnett-Smith, J. et al., 2001, Exp Cell Res. 266:292-302; Chrissobolis, S. and Sobey, C. G., 2001, Circ Res. 88:774-779; Honjo, M. et al., 2001, Invest Ophthalmol Vis Sci. 42:137-144; Takahara, A. et al., 2003, Eur J Pharmacol. 460:51-57; Fournier, A. E. et al., 2003, J Neurosci. 23:1416-1423; Rikitake, Y. et al., 2005, Stroke. 36:2251-2257; Slotta, J. E. et al. 2006, Inflamm Res. 55:364-367; Ying, H. et al., 2006, Mol Cancer Ther. 5:2158-2164]. The correlation between small molecule inhibition of Rho kinases and changes in cell behavior both in vitro and in vivo (e.g., vascular smooth muscle relaxation, bronchial smooth muscle relaxation, inhibition of immune and inflammatory cell migration, inhibition of tumor cell migration, inhibition of experimentally induced fibrosis, promotion of neural regenerative activity) supports the notion that Rho kinases are significant pharmaceutical targets for a wide range of therapeutic indications. In addition, it is now more appreciated that some of the “pleiotropic” and beneficial cardiovascular effects of clinically useful HMG Coenzyme A reductase inhibitors (i.e., the “statin” drug class) are a consequence of decreased Rho, and therefore decreased Rho kinase, activity, especially in endothelial cells [Eto, M. et al., 2002, Circulation. 105:1756-1759; Rikitake, Y. and Liao, J. K., 2005, Circ Res. 97:1232-1235; Kozai, T. et al., 2005, Cardiovasc Res. 68:475-482; Girgis, R. E. et al., 2007, Am J Physiol Lung Cell Mol Physiol. 292:L1105-L110]. Interestingly, Rho kinase inhibition has been recently implicated in the enhanced survival and cloning efficiency of dissociated human embryonic stem cells, which suggests the utility of Rho kinase inhibitors for stem cell therapies [Watanabe, K. et al., 2007, Nat Biotechnol. 25:681-686].

Novel compounds and pharmaceutical compositions, certain of which have been found to inhibit Rho kinase have been discovered, together with methods of synthesizing and using the compounds including methods for the treatment of Rho kinase-mediated diseases in a patient by administering the compounds.

The present invention discloses a class of compounds, certain of which may be useful in treating Rho kinase-mediated disorders and conditions, defined by structural Formula I:

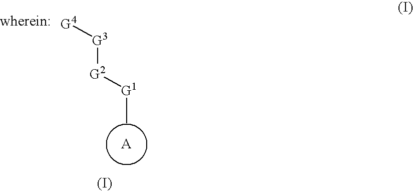

A is optionally substituted heteroaryl;

G¹ is optionally substituted 5-membered heteroaryl;

G² is selected from the group consisting of (CR^{a}R^{b})ₘZ(CR^{c}R^{d})ₚ and null;

m and p are independently 0, 1, 2, 3, or 4;

Z is selected from the group consisting of 0, N(R¹), S(O)ₙ, N(R^{e})CO, CON(R^{e}), N(R^{e})SO₂, SO₂N(R^{e}), carbonyl, optionally substituted cycloalkyl, and null;

R^{c} is selected from the group consisting of hydrogen and C₁-C₄ alkyl;

n is 0, 1 or 2;

R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;

G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;

G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and

R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted.

Certain compounds according to the present invention possess useful Rho kinase inhibiting activity, and may be used in the treatment or prophylaxis of a disease or condition in which Rho kinase plays an active role. Thus, in broad aspect, the certain embodiments of the present invention also provide pharmaceutical compositions comprising one or more compounds disclosed herein together with a pharmaceutically acceptable carrier, as well as methods of making and using the compounds and compositions. Certain embodiments of the present invention provide methods for inhibiting Rho kinase. Other embodiments of the present invention provide methods for treating a Rho kinase-mediated disorder in a patient in need of such treatment, comprising administering to said patient a therapeutically effective amount of a compound or composition according to the present invention. The present invention also contemplates the use of certain compounds disclosed herein for use in the manufacture of a medicament for the treatment of a disease or condition ameliorated by the inhibition Rho kinase.

In certain embodiments, the compounds of the present invention have structural Formula II or structural Formula III

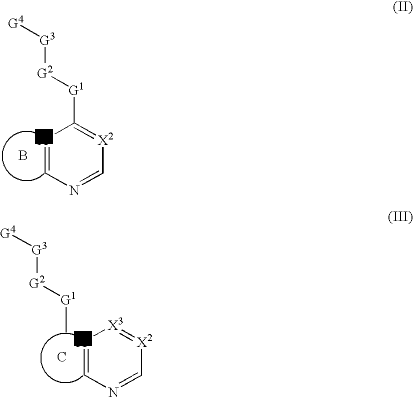

wherein:

X¹ is C(R²) or N;

X² is C(R³) or N;

X³is C(R⁴) or N;

B is selected from the group consisting of optionally substituted 5-membered heteroaryl and null;

C is optionally substituted 5-membered heteroaryl;

G¹ is optionally substituted 5-membered heteroaryl;

G² is selected from the group consisting of (CR^{a}R^{b})ₘZ(CR^{c}R^{d})ₚ and null;

m is 1, 2, 3, or 4;

p is 0, 1, 2, 3, or 4;

Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;

R^{e} is selected from the group consisting of hydrogen and C₁-C₄ alkyl;

n is 0, 1 or 2;

R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;

G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;

G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and

R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted; and

R², R³, and R⁴ are each independently selected from the group consisting of acyl, lower alkenyl, alkynyl, lower alkoxy, lower alkoxyalkyl, lower alkyl, alkylthio, amido, amino, aminoalkyl, aminocarbonyl, aralkyl, arylcarbonyl, arylsulfonyl, cycloalkyl, cycloalkylalkyl, carboxyl, cycloalkenyl, halo, haloalkyl, hydroxyalkyl, heteroaryl, heteroaralkyl, heterocycloalkyl, heterocycloalkylalkyl and hydrogen, any of which may be optionally substituted.

In further embodiments, the compounds of the present invention have structural Formula IV or structural Formula V

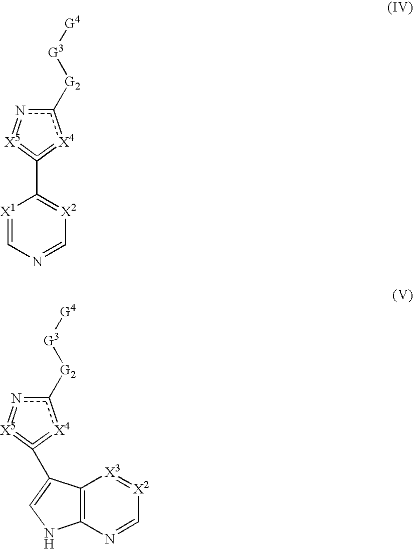

wherein:

X¹ is C(R²) or N;

X² is C(R³) or N;

X³is C(R⁴) or N;

X⁴ is selected from the group consisting of C(R⁵), O, N, and S;

X⁵is selected from the group consisting of C(R⁶) and N;

G² is selected from the group consisting of (CR^{a}R^{b})ₘZ(CR^{c}R^{d})ₚ and null;

m is 1, 2, 3, or 4;

p is 0, 1, 2, 3, or 4;

Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;

R^{e} is selected from the group consisting of hydrogen and C₁-C₄ alkyl;

n is 0, 1 or 2;

R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;

G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;

G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and

R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted;

R², R³, and R⁴ are each independently selected from the group consisting of acyl, lower alkenyl, alkynyl, lower alkoxy, lower alkoxyalkyl, lower alkyl, alkylthio, amido, amino, aminoalkyl, aminocarbonyl, aralkyl, arylcarbonyl, arylsulfonyl, cycloalkyl, cycloalkylalkyl, carboxyl, cycloalkenyl, halo, haloalkyl, hydroxyalkyl, heteroaryl, heteroaralkyl, heterocycloalkyl, heterocycloalkylalkyl and hydrogen, any of which may be optionally substituted; and

R⁵ and R⁶ are each independently selected from the group consisting of lower alkenyl, alkynyl, lower alkoxy, lower alkoxyalkyl, lower alkyl, alkylthio, amino, aminoalkyl, aminocarbonyl, arylcarbonyl, cycloalkyl, carboxy, haloalkyl, hydroxyalkyl, heterocycloalkyl and hydrogen, any of which may be optionally substituted.

In yet further embodiments,

G² is (CR^{a}R^{b})ₘZ(R^{c}R^{d})ₚ;

p is 0;

m is 1;

Z is N(R^{e})CO; and

R^{e} is hydrogen.

In yet further embodiments,

R^{a} is selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl; and

R^{b} is hydrogen.

In yet further embodiments,

R^{a} is selected from the group consisting of hydrogen, lower alkyl, aminoalkyl, amidoalkyl, carboxylalkyl, heterocycloalkylalkyl, and heteroarylalkyl.

In yet further embodiments,

X⁴ is selected from the group consisting of C(R⁵) and N;

G³ is selected from the group consisting of aryl and heteroaryl, any of which may be optionally substituted; and

R⁵ and R⁶ are hydrogen.

In yet further embodiments,

X¹ is C(R²);

X² is C(R³);

X³ is C(R⁴); and

R², R³, and R⁴ are hydrogen.

In yet further embodiments, G³ has a formula selected from the group consisting of:

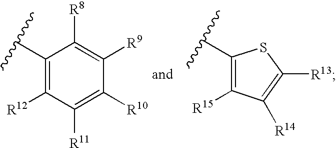

R⁸, R⁹, R¹¹, R¹², R¹³, R¹⁴, and R¹⁵ are independently selected from the group consisting of hydrogen, and at least one substituent selected from the group consisting of acyl, O-acyl, amino, amido, aryl, arylalkoxy, lower alkyl, lower alkoxy, carboxyl, cycloalkyl, hydroxy, halo, lower haloalkyl, lower haloalkoxy, heterocycloalkyl, heteroaryl, nitro, phenoxy and sulfonyl; and

R¹⁰ is selected from the group consisting of acyl, O-acyl, amino, amido, lower alkyl, C₂-C₆ alkoxy, carboxyl, cycloalkyl, fluorine, bromine, iodine, lower haloalkyl, lower haloalkoxy, heterocycloalkyl, heteroaryl, hydrogen, hydroxy, phenoxy, nitro and sulfonyl.

In certain embodiments, compounds of structural Formulas I-V may find use in the inhibition of Rho kinase for the treatment of disease.

In certain embodiments, compounds of structural Formulas I-V may be administered in combination with at least one other therapeutic agent.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—N(R)₂ group with R as defined herein. The term “N-amido” as used herein, alone or in combination, refers to a RC(═O)N(R′)— group, with R and R′ as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —N(R)(R′) or —N⁺(R)(R′)(R″), wherein R, R′ and R″ are independently selected from the group consisting of hydrogen, alkyl, acyl, heteroalkyl, aryl, cycloalkyl, heteroaryl, and heterocycloalkyl, any of which may themselves be optionally substituted.

The term “amino acid,” as used herein, alone or in combination, means a substituent of the form —NRCH(R′)C(O)OH, wherein R is typically hydrogen, but may be cyclized with N (for example, as in the case of the amino acid proline), and R′ is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogues.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxyl,” as used herein, refers to —C(O)OH, O-carboxy, C-carboxy, or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxyl group bridging two moieties linked at carbon atoms.

The term “ether,” as used herein, alone or in combination, typically refers to an oxy group bridging two moieties linked at carbon atoms. “Ether” may also include polyethers, such as, for example, —RO(CH₂)₂O(CH₂)₂O(CH₂)₂OR′, —RO(CH₂)₂O(CH₂)₂OR′, —RO(CH₂)₂OR′, and —RO(CH₂)₂OH.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃. The term heteroalkyl may include ethers.

The term “heteroaryl,” as used herein, alone or in combination, refers to 3 to 7 membered unsaturated heteromonocyclic rings, or fused polycyclic rings in which at least one of the fused rings is unsaturated, wherein at least one atom is selected from the group consisting of O, S, and N. In certain embodiments, said heteroaryl will comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one heteroatom as ring members, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sugars, sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycloalkyl groups may be optionally substituted unless specifically prohibited.

The term “hydrazinyl” as used herein, alone or in combination, refers to two amino groups joined by a single bond, i.e., —N—N—.

The term “hydroxamic acid” as used herein, refers to —C(O)ON(R)O(R′), wherein R and R′ are as defined herein, or the corresponding “hydroxamate” anion, including any corresponding hydroxamic acid salt. Hydroxamate also includes reverse hydroxamates of the form —ON(R)O(O)CR′.

The term “hydroxy,” or, equivalently, “hydroxyl,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(O)O—] in which one or more of the hydroxyl groups has been replaced by nitrogen, amino, or amido.

The term “phosphonate” as used herein, alone or in combination, refers to a group of the form ROP(OR═)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refers to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. A “null” group occurring between two other groups may also be understood to be a collapsing of flanking groups. For example, if in —(CH₂)ₛG¹G²G³, the element G were null, said group would become —(CH₂)ₛG¹G³.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, hydroxyl, halogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers, including keto-enol tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“Rho kinase inhibitor” is used herein to refer to a compound that exhibits an IC₅₀ with respect to Rho kinase activity of no more than about 100 μM and more typically not more than about 50 μM, as measured in the Rho kinase assay described generally hereinbelow. “IC₅₀” is that concentration of inhibitor which reduces the activity of an enzyme (e.g., Rho kinase) to half-maximal level. Certain representative compounds of the present invention have been discovered to exhibit inhibition against Rho kinase. In certain embodiments, compounds will exhibit an IC₅₀ with respect to Rho kinase of no more than about 10 μM; in further embodiments, compounds will exhibit an IC₅₀ with respect to Rho kinase of no more than about 5 μM; in yet further embodiments, compounds will exhibit an IC₅₀ with respect to Rho kinase of not more than about 1 μM, as measured in the Rho kinase assay described herein. In yet further embodiments, compounds will exhibit an IC₅₀ with respect to Rho kinase of not more than about 200 nM.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain of the present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds of the present invention, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating Rho kinase-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of Rho kinase-mediated disorders.

Compounds of the subject invention may be useful in treating Rho kinase-mediated disease, disorders and conditions. In certain embodiments, said compounds may find use in treating acute and chronic pain and inflammation. The compounds of the present invention may be useful to treat patients with neuropathy, neuropathic pain, or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds may also be useful in the treatment of pain associated with acute herpes zoster (shingles), postherpetic neuralgia (PHN), and associated pain syndromes such as ocular pain. The compounds may further be useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds may also be useful for the treatment of pain-related disorders such as tactile allodynia and hyperalgesia. The pain may be somatogenic (either nociceptive or neuropathic), acute and/or chronic. The Rho kinase inhibitors of the subject invention may also be useful in conditions where NSAIDs, morphine or fentanyl opiates and/or other opioid analgesics would traditionally be administered.

Furthermore, compounds of the subject invention may be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods of the present invention may be useful in the treatment or prevention of drug withdrawal symptoms, for example treatment or prevention of symptoms of withdrawal from opiate, alcohol, or tobacco addiction.

In addition, compounds of the subject invention may be used to treat insulin resistance and other metabolic disorders such as atherosclerosis that are typically associated with an exaggerated inflammatory signaling.

The present invention encompasses therapeutic methods using novel selective Rho kinase inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; asthma-related diseases such as airway hyperreactivity and small airway disease; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchiolitis, bronchioectasis, cystic fibrosis, pigeon fancier's disease, farmer's lung, acute respiratory distress syndrome, pneumonia, pneumonitis, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus, hypoxia, dyspnea, hypercapnea, hyperinflation, hypoxemia, and cough. Further, compounds disclosed herein would find use in the treatment of allergic disorders such as delayed type hypersensitivity reaction, allergic contact dermatitis, allergic rhinitis, and chronic sinusitis.

Other disorders or conditions which may be treated by the compounds of the present invention include inflammation and related disorders. The compounds of the present invention may be useful as anti-inflammatory agents with the additional benefit of having significantly less harmful side effects. The compounds may be useful to treat arthritis, including but not limited to rheumatoid arthritis, spondyloarthropathies, gouty arthritis, osteoarthritis, juvenile arthritis, acute rheumatic arthritis, enteropathic arthritis, neuropathic arthritis, psoriatic arthritis, reactive arthritis (Reiter's syndrome), and pyogenic arthritis, and autoimmune diseases, including systemic lupus erythematosus, hemolytic syndromes, autoimmune hepatitis, autoimmune neuropathy, vitiglio (autoimmune thyroiditis), Hashimoto's thyroiditis, anemias, myositis including polymyositis, alopecia greata, Goodpasture's syndrome, hypophytis, and pulmonary fibrosis.

The compounds may also be useful in treating osteoporosis and other related bone disorders.

These compounds may also be used to treat gastrointestinal conditions such as reflux esophagitis, diarrhea, inflammatory bowel disease, Crohn's disease, gastritis, irritable bowel syndrome, Graves' disease (hyperthyroidism), necrotizing enterocolitis,and ulcerative colitis. The compounds may also be used in the treatment of pulmonary inflammation, such as that associated with viral infections and cystic fibrosis.

In addition, compounds of invention may also be useful in organ transplant patients either alone or in combination with conventional immunomodulators. Examples of conditions to be treated in said patients include graft vs. host reaction (i.e., graft vs. host disease), allograft rejections (e.g., acute allograft rejection, and chronic allograft rejection), transplant reperfusion injury, and early transplantation rejection (e.g., acute allograft rejection).

Yet further, the compounds of the invention may be useful in the treatment of pruritis and vitaligo.

The compounds of the present invention may also be useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Langerhans' cell histiocytosis, glomerulonephritis, reperfusion injury, pancreatitis, interstitial cystitis, Behcet's syndrome, polymyositis, gingivitis, periodontis, hypersensitivity, swelling occurring after injury, ischemias including myocardial ischemia, cardiovascular ischemia, and ischemia secondary to cardiac arrest, cirrhosis, septic shock, endotoxic shock, gram negative sepsis, toxic shock syndrome, stroke, ischemia reperfusion injury, multi-organ dysfunction, restenosis including restenosis following coronary bypass surgery, and the like.

The compounds of the subject invention may also be useful for the treatment of certain diseases and disorders of the nervous system. Central nervous system disorders in which Rho kinase inhibition may be useful include cortical dementias including Alzheimer's disease and mild cognitive impairment (MCI), central nervous system damage resulting from stroke, ischemias including cerebral ischemia (both focal ischemia, thrombotic stroke and global ischemia (for example, secondary to cardiac arrest), and trauma. Neurodegenerative disorders in which Rho kinase inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia (e.g. pre-senile dementia), and AIDS-related dementia, cachexia, Sydenham's chorea, Huntington's disease, Parkinson's Disease, amyotrophic lateral sclerosis (ALS), multiple sclerosis, Korsakoff's syndrome, and imbecility relating to a cerebral vessel disorder. Further disorders in which Rho kinase inhibition might prove useful include neuropathies of the central and peripheral nervous system (including, for example, IgA neuropathy, membranous neuropathy and idiopathic neuropathy), chronic inflammatory demyelinating polyneuropathy, transverse myelitis, Gullain-Barre disease, encephalitis, and cancers of the nervous system. Disorders of CNS function in which Rho kinase inhibitors may find use include sleeping disorders, schizophrenia, depression, depression or other symptoms associated with Premenstrual Syndrome (PMS), and anxiety.

Furthermore, the compounds of the present invention may also be useful in inhibiting Rho kinase activity for the amelioration of systemic disorders including septic and/or toxic hemorrhagic shock induced by a wide variety of agents; as a therapy with cytokines such as TNF, IL-1 and IL-2; and as an adjuvant to short term immunosuppression in transplant therapy.

Still other disorders or conditions which may be treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, leukemia, lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods may also be used to treat the fibrosis which occurs with radiation therapy. The present compounds and methods may be used to treat subjects having adenomatous polyps, including those with familial adenomatous polyposis (FAP). Additionally, the present compounds and methods may be used to prevent polyps from forming in patients at risk of FAP.

The compounds of the subject invention may be used in the treatment of ophthalmic diseases, such as dry eye, glaucoma, corneal neovascularization, optic neuritis, Sjogren's syndrome, retinal ganglion degeneration, ocular ischemia, retinitis, retinopathies, uveitis, ocular photophobia, and of inflammation and pain associated with acute injury to the eye tissue. Specifically, the compounds may be used to treat glaucomatous retinopathy and/or diabetic retinopathy. The compounds may also be used to treat post-operative inflammation or pain as from ophthalmic surgery such as cataract surgery and refractive surgery.

The compounds of the subject invention may be used in the treatment of menstrual cramps, dysmenorrhea, premature labor, endometriosis, tendonitis, bursitis, skin-related conditions such as psoriasis, eczema, burns, sunburn, dermatitis, pancreatitis, hepatitis, lichen planus, scleritis, scleroderma, dermatomyositis, and the like. Other conditions in which the compounds of the subject invention may be used include diabetes (type I or type II), myocarditis, pathological angiogenesis, and aortic aneurysm.

Moreover, compounds of the subject invention may be used in the treatment of cardiovascular disease, such as angina, coronary artery vasospasm, myocardial infarction, coronary ischemia, congestive heart failure, cardiac allograft vasculopathy, vein graft disease and vascular restenosis, ischemic reperfusion injury, cerebral artery vasospasm, stroke, cerebral ischemia, essential hypertension, pulmonary hypertension, renal hypertension and other secondary hypertensive disorders, atherosclerosis and erectile dysfunction.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Differentiated cells produced from hES cells may be useful for treating degenerative diseases whose symptoms are caused by loss of a few particular cell types. Specific types of neurons have been generated from mouse ES (mES) cells, and similar selective differentiation methods have been applied to hES cells. However, hES cells have been technically much harder to culture than mES cells, showing problematic properties such as slow growth and insensitivity to the trophic substance leukemia inhibitory factor (LIF). In addition, hES cells are vulnerable to apoptosis upon cellular detachment and dissociation. They undergo massive cell death particularly after complete dissociation, and the cloning efficiency of dissociated hES cells is generally ≦1%. Thus, hES cells are difficult, if not impossible, to use in dissociation culture, which is important for such procedures as clonal isolation following gene transfer and differentiation induction. Poor survival of human embryonic stem (hES) cells after cell dissociation is an obstacle to research, hindering manipulations such as subcloning.

Many methods exist for the production or derivation of hES cells. For example, histocompatible parthenogenetic human embryonic stem cells (phESC) may be derived from human parthenogenetic blastocysts. The utility of Rho kinase inhibitors disclosed above, and the methods below, would be expected to be applicable to any hES cells demonstrating typcal hES cell morphology and/or properties, regardless of origin.

Recent evidence suggests that addition of selective inhibitors of Rho kinase may enable hES cells to grow and differentiate as mES cells do under unfavorable culture conditions such as dissociation and suspension. Rho kinase inhibition has been shown to markedly diminish dissociation-induced apoptosis, increase cloning efficiency (from 1% to -27%) and facilitate subcloning after gene transfer in hES cells. The improvement in cloning efficiency conferred Rho kinase inhibition may be particularly advantageous for isolating relatively rare clones (e.g., those for homologous recombination) and also for recloning hES cells to obtain a uniform cell quality. Furthermore, dissociated hES cells treated with selective inhibitors of Rho kinase are protected from apoptosis even in serum-free suspension (SFEB) culture, form floating aggregates, and survive and differentiate, as do SFEB-cultured mouse ES cells.

Accordingly, the invention contemplates the use of certain compounds and compositions disclosed herein: for reduction of apoptosis of human embryonic stem cells; for increasing survival of human embryonic stem cells; for increasing cloning efficiency of human embryonic stem cells after gene transfer; and for enhancing differentiation of cultured human embryonic stem cells. In further embodiments, said prevention of apoptosis of human embryonic stem cells and/or said increasing of survival of human embryonic stem cells occurs in dissociated culture, such as, for example, serum-free suspension (SFEB) culture.

Besides being useful for human treatment, the compounds and formulations of the present invention are also useful for veterinary treatment of companion animals, exotic animals and farm animals, including mammals, rodents, and the like. More preferred animals include horses, dogs, and cats.

### General Synthetic Methods for Preparing Compounds

The following schemes can be used to practice the present invention. 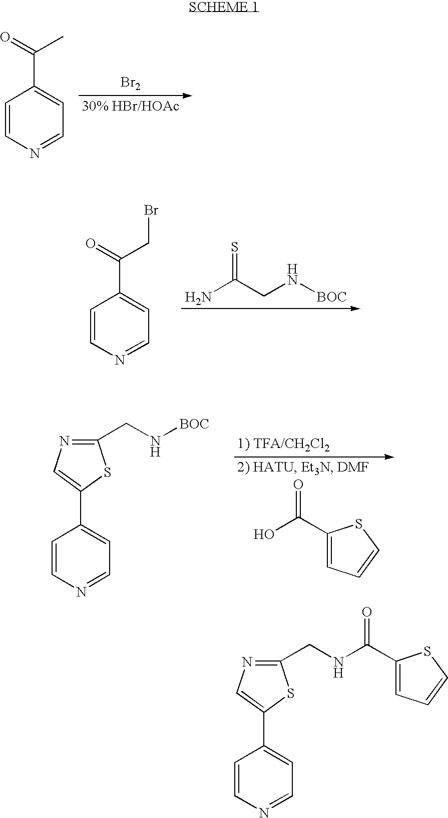
Example 1 can be synthesized using the following general synthetic procedure set forth in Scheme 1. 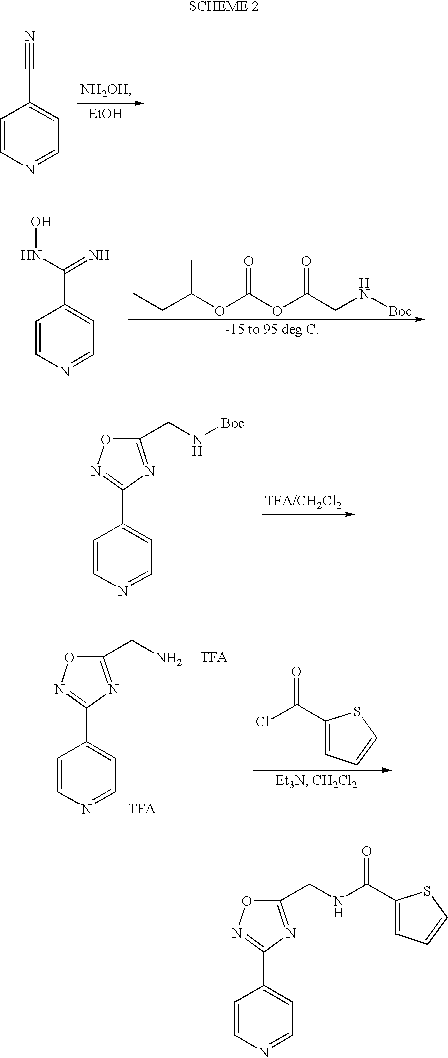
Examples 2-4 can be synthesized using the following general synthetic procedure set forth in Scheme 2. 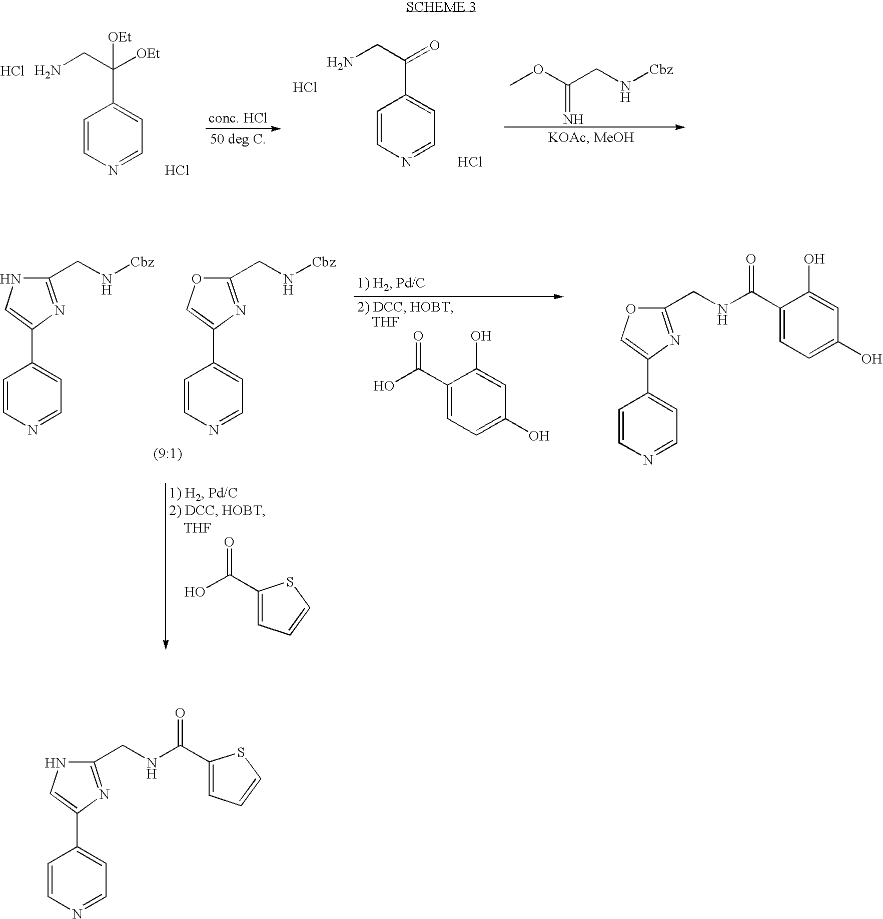
Examples 5-8 can be synthesized using the following general synthetic procedure set forth in Scheme 3. 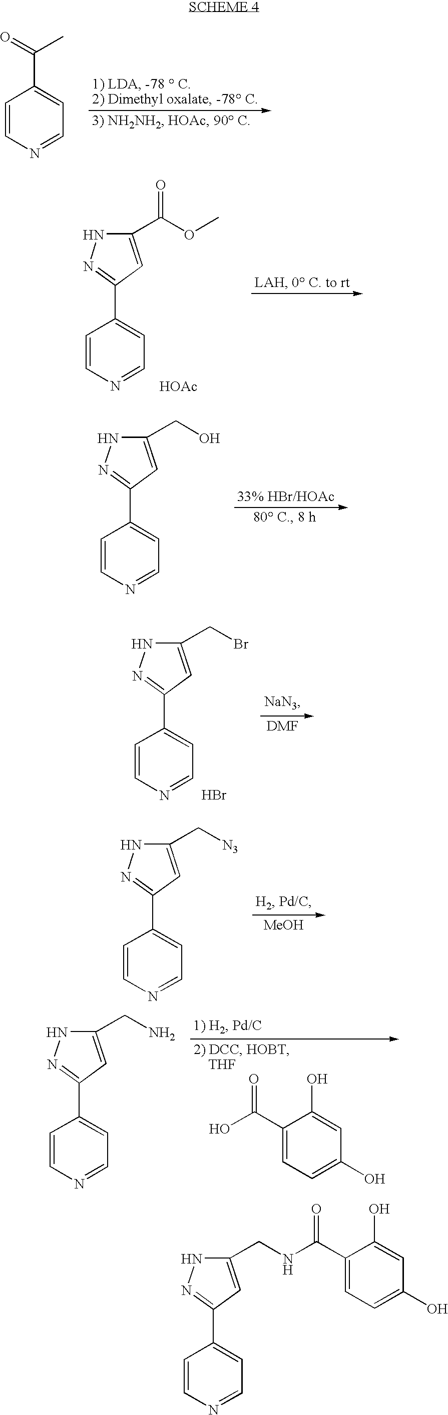
Examples 9-11 can be synthesized using the following general synthetic procedure set forth in Scheme 4. 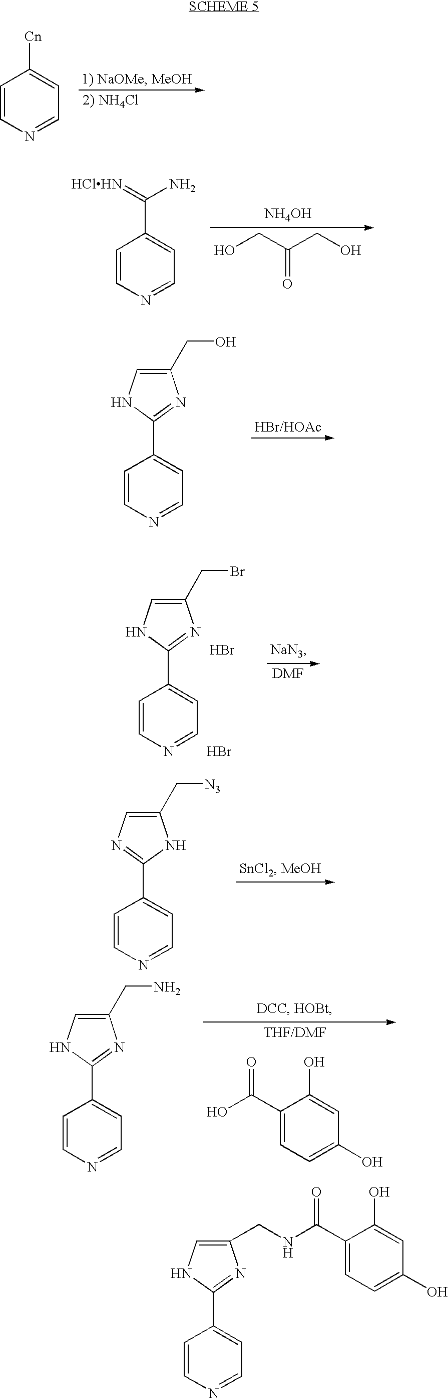
Examples 12-14 can be synthesized using the following general synthetic procedure set forth in Scheme 5. 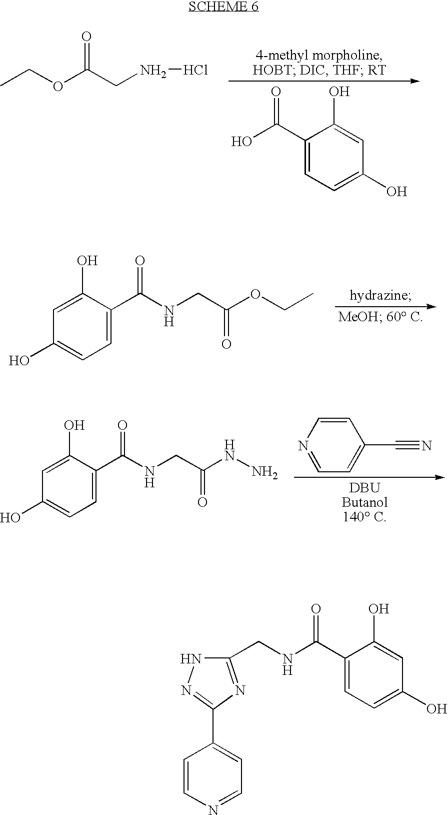
Example 15 can be synthesized using the following general synthetic procedure set forth in Scheme 6. 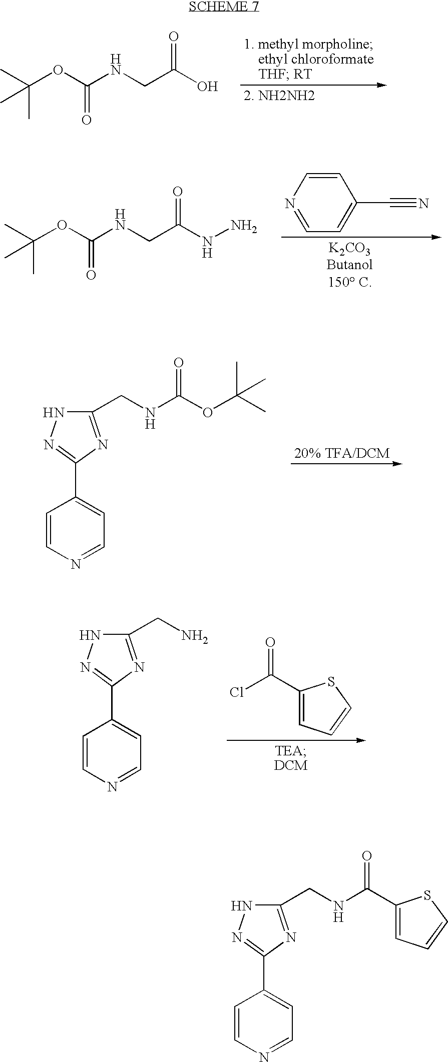
Examples 16-36 can be synthesized using the following general synthetic procedure set forth in Scheme 7. 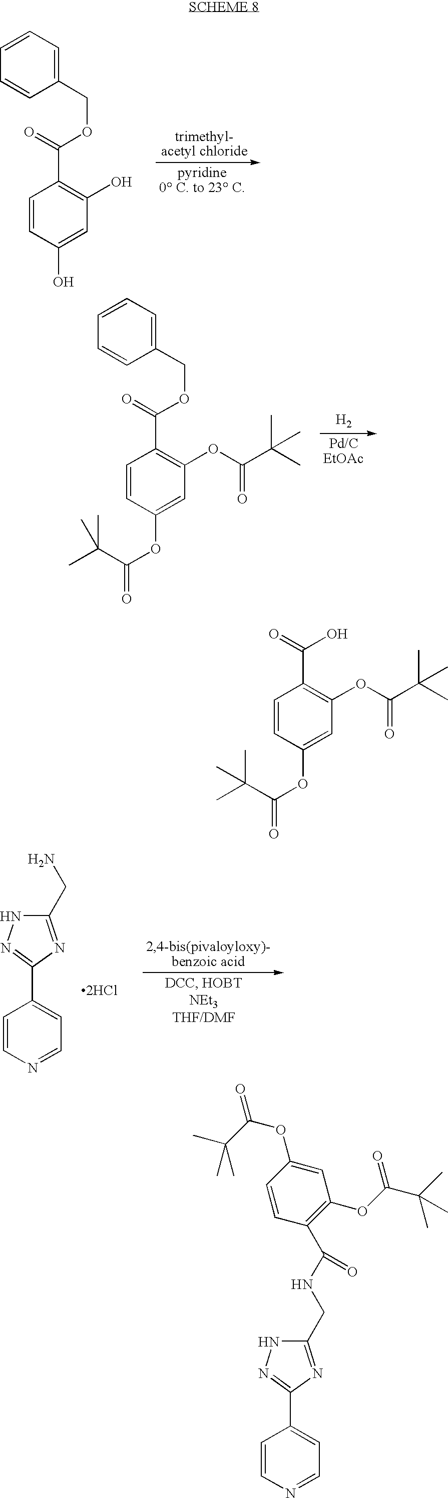
Example 37 can be synthesized using the following general synthetic procedure set forth in Scheme 8. 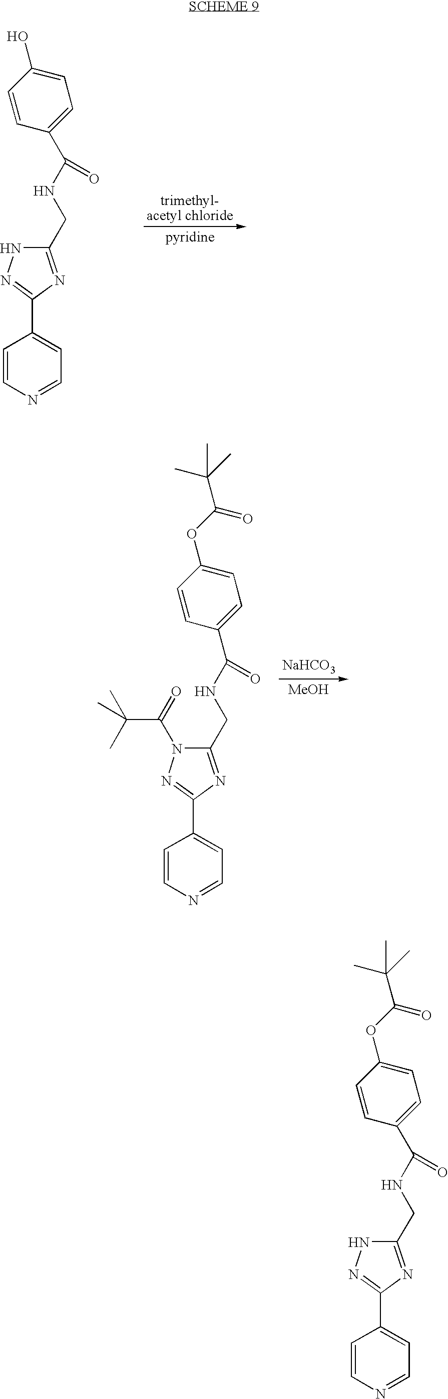
Examples 38-39 can be synthesized using the following general synthetic procedure set forth in Scheme 9. 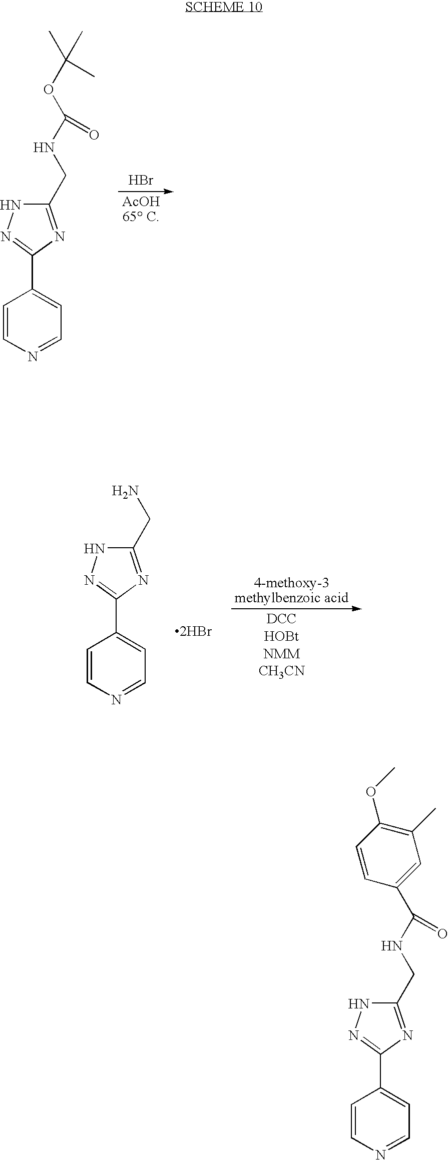
Examples 40-56 can be synthesized using the following general synthetic procedure set forth in Scheme 10. 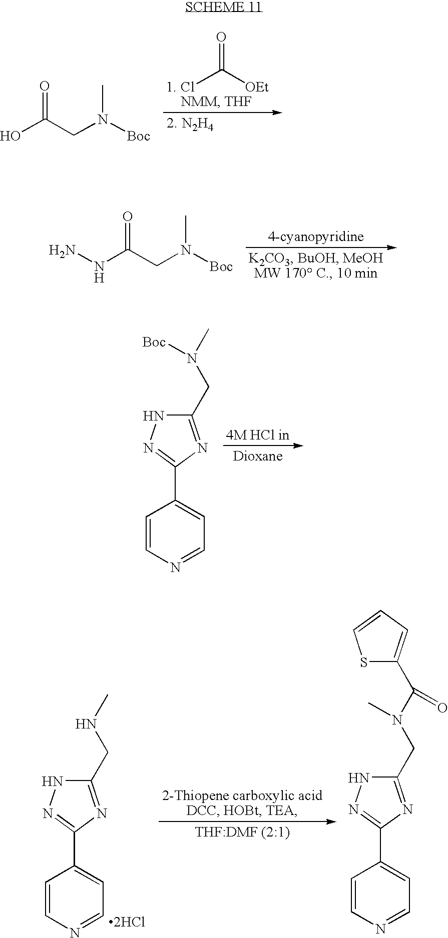
Examples 57-58 can be synthesized using the following general synthetic procedure set forth in Scheme 11. 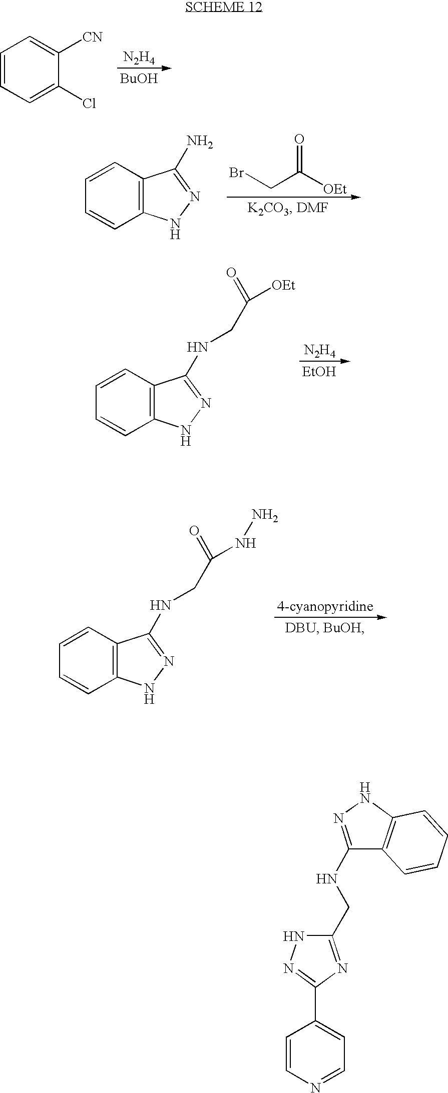
Example 59 can be synthesized using the following general synthetic procedure set forth in Scheme 12. 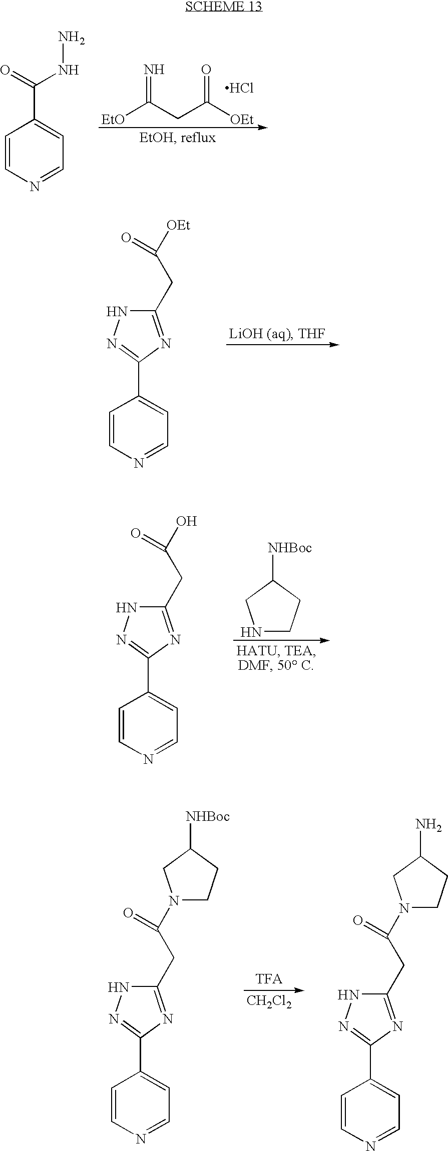
Examples 60-61 can be synthesized using the following general synthetic procedure set forth in Scheme 13. 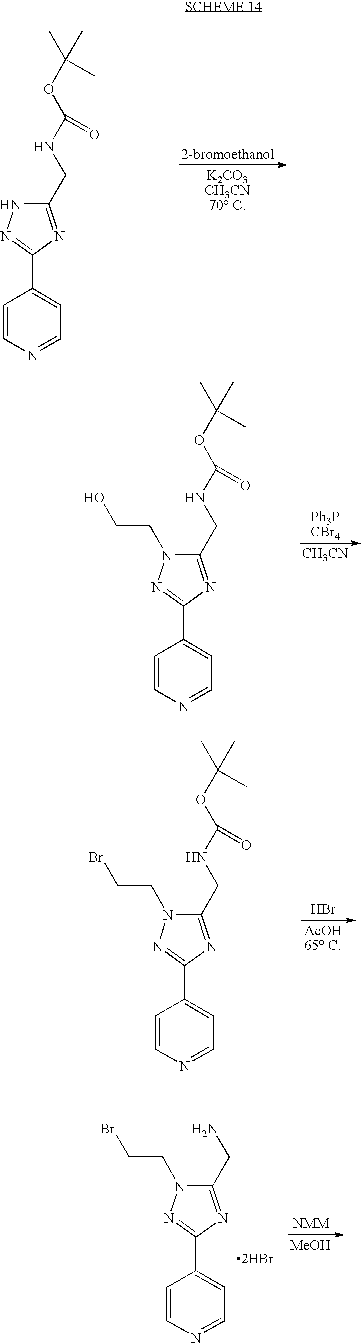 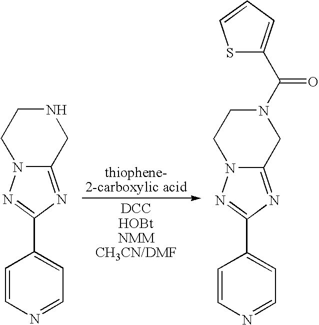
Example 62 can be synthesized using the following general synthetic procedure set forth in Scheme 14. 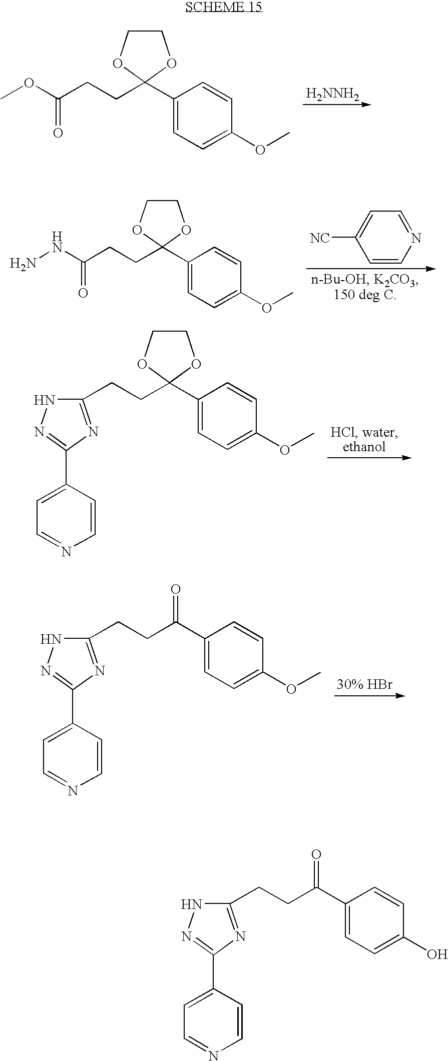
Examples 63-64 can be synthesized using the following general synthetic procedure set forth in Scheme 15. 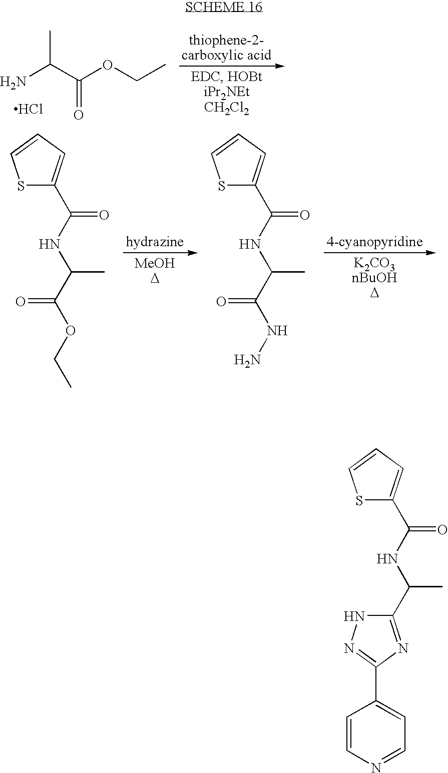
Example 65 can be synthesized using the following general synthetic procedure set forth in Scheme 16. 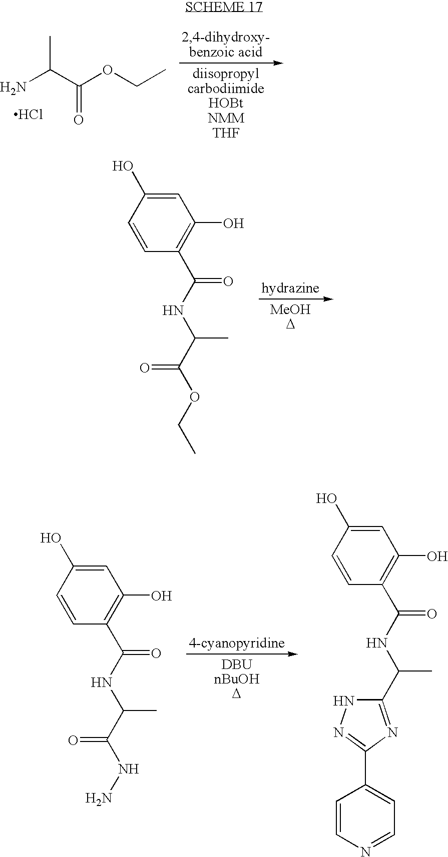
Example 66 can be synthesized using the following general synthetic procedure set forth in Scheme 17. 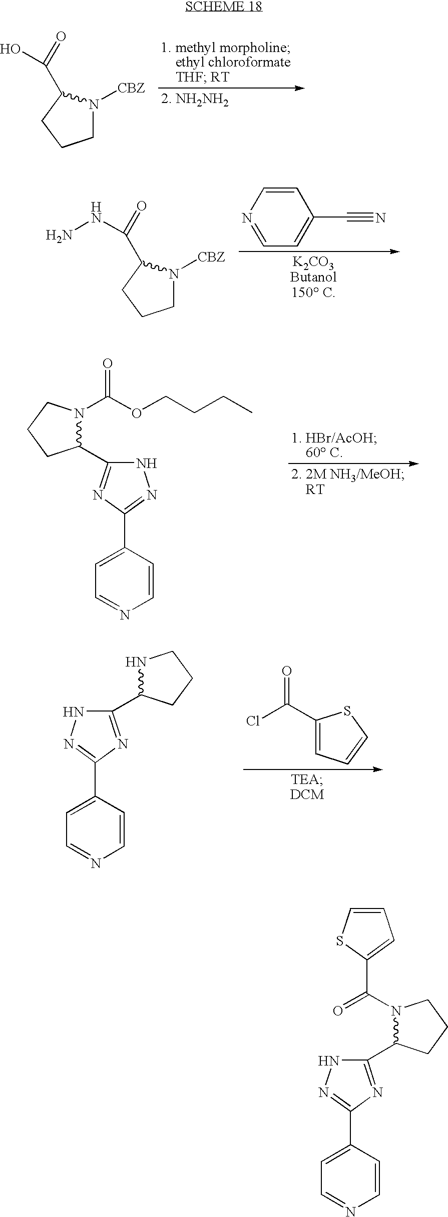
Example 67 can be synthesized using the following general synthetic procedure set forth in Scheme 18. 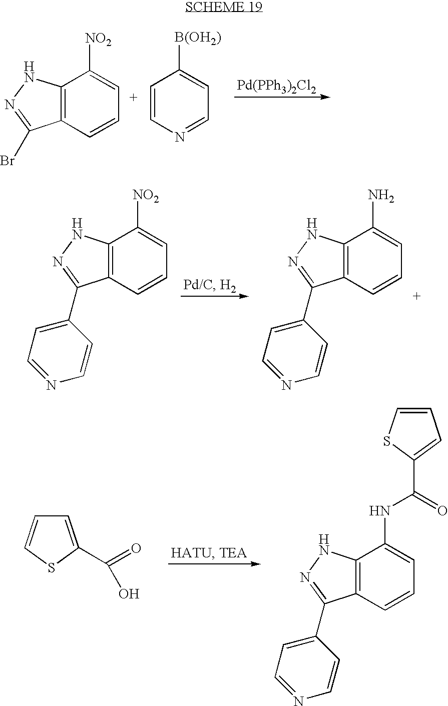
Examples 68 can be synthesized using the following general synthetic procedure set forth in Scheme 19. 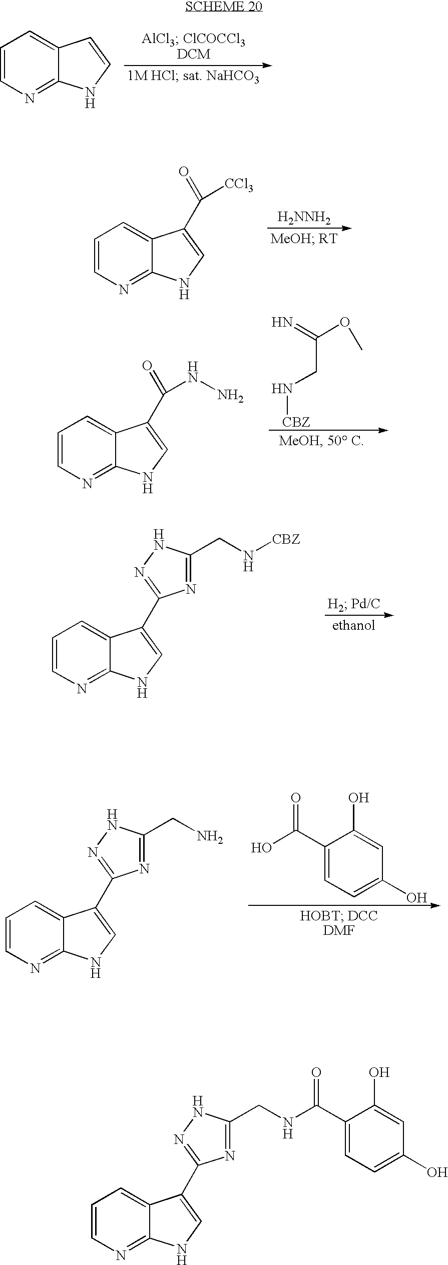
Examples 69-72 can be synthesized using the following general synthetic procedure set forth in Scheme 20. 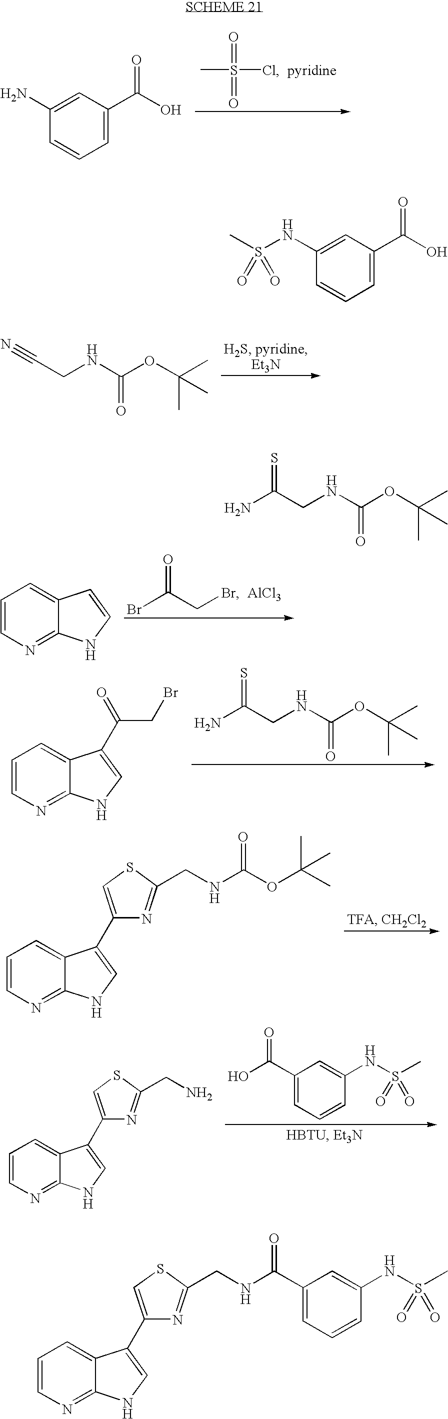
Examples 73-75 can be synthesized using the following general synthetic procedure set forth in Scheme 21. 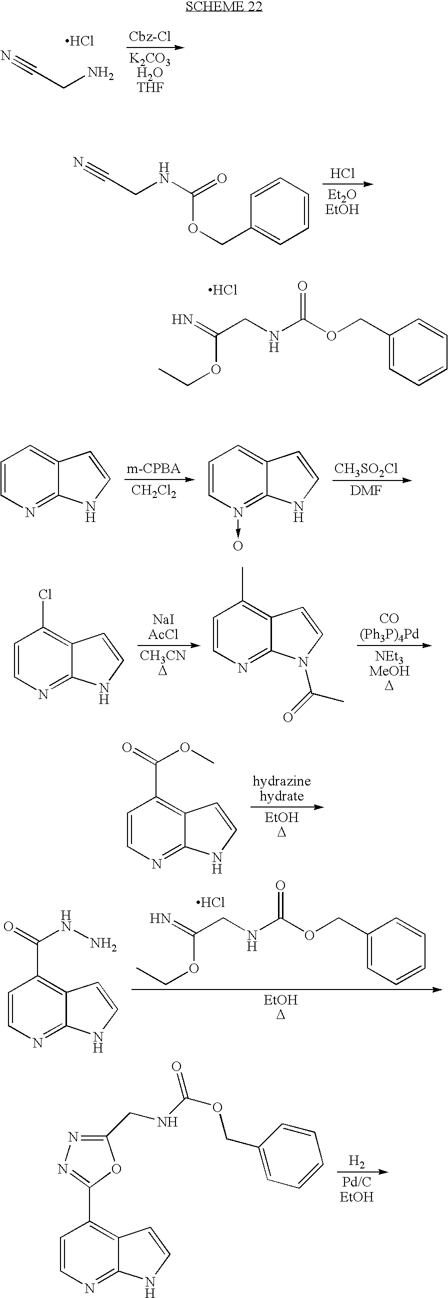 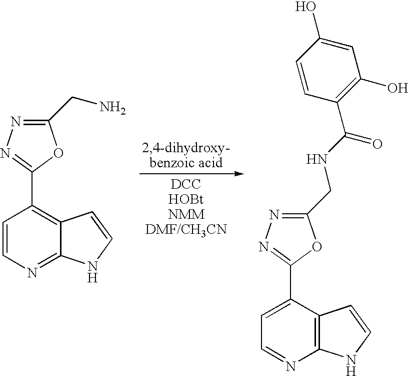
Example 76 can be synthesized using the following general synthetic procedure set forth in Scheme 22.
The invention is further illustrated by the following examples.

### EXAMPLE 1

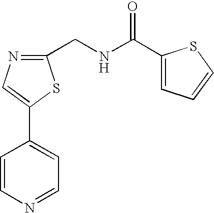

### N-((5-(pyridin-4-yl)thiazol-2-yl)methyl)thiophene-2-carboxamide:

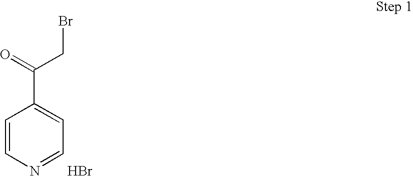

### 2-bromo-1-(pyridin-4-yl)ethanone hydrobromide:

Bromine (2.32 mL, 45.1 mmol) was added dropwise to a solution of 1-(pyridin-4-yl)ethanone and 30% HBr/Acetic acid (50 mL) in a 250 mL round bottom flask cooled to 0° C. in an ice bath. The resulting solution decolorized immediately. After 5-10 min. a fine yellow precipitate formed. The reaction was allowed to react for 1 hour while the temperature was maintained at 0° C. Work-up: the reaction was diluted with ethyl acetate (100 mL), and the resulting yellow solid was filtered and rinsed with ethyl acetate, yielding the product as an HBr salt (9.89 g, 87% yield, >90% pure). ¹H NMR (400 MHz, DMSO-d₆) δ: 10.90 (bs, 1H), 9.02 (d, 2H), 8.19 (d, 2H), 5.06 (s, 2H).

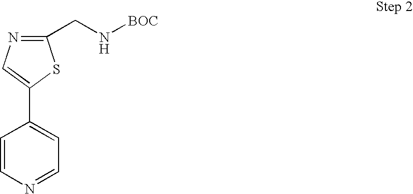

### tert-butyl(5-(pyridin-4-yl)thiazol-2-yl)methylcarbamate

A 50 mL round bottom flask, was charged with 2-bromo-1-(pyridin-4-yl)ethanone hydrobromide (558 mg, 2.0 mmol), tert-butyl 2-amino-2-thioxoethylcarbamate (418 mg, 2.2 mmol), and ethanol (800 mL). A red solution formed immediately. The resulting solution was heated to 70° C. and allowed to react for 25 min at this temperature. Work-up: the reaction was diluted with ethyl acetate (50 mL), washed with 1N NaOH (color changed from red to yellow), dried with MgSO₄, filtered, concentrated, and purified by column chromatography (0 to50%) acetonitrile/methylene chloride. There were many mixed fractions. The mass of those that were pure was 133mg, 19% yield. ¹H NMR (400 MHz, CDCl₃) δ 8.65 (d, 2H), 7.76 (d, 2H), 7.66 (s, 1H), 5.36 (bm, 1H), 4.67 (m, 2H), 1.48 (s, 9H).

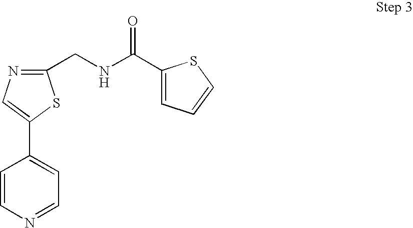

### N-((5-(pyridin-4-yl)thiazol-2-yl)methyl)thiophene-2-carboxamide

A 10 mL vial was charged with tert-butyl (5-(pyridin-4-yl)thiazol-2-yl)methylcarbamate (100 mg, 0.343 mmol), and 30% TFA/methylene chloride (3 mL). The resulting solution was stirred for 15 min. at room temperature, during which time the color changed from red to yellow. The reaction mixture was concentrated to an oil, azeotroped with toluene (2×), and then used in the next step with further purification. The crude TFA salt was dissolved in DMF (1 mL), to which thiophene-2-carboxylic acid (48.3 mg, 0.377 mmol), Et₃N (191 μL, 1.372 mmol), and HATU (156 mg, 0.412 mmol) were added in that order. The resulting mixture was stirred overnight at room temperature. The reaction progress was monitored by TLC (40% acetonitrile/methylene chloride). Work-up: the mixture was concentrated and purified by C18 reverse phase semi-preparative HPLC, giving 85 mg (82% yield) of the title compound. ¹H NMR (400 MHz, DMSO-d₆) δ 9.51 (m, 1H), 8.78 (d, 2H), 8.67 (s, 1H), 8.21 (d, 2H), 7.84 (m, 2H), 7.18 (m, 1H), 4.78 (d, 2H). LCMS (M+1)⁺: 301.62.

### EXAMPLE 2

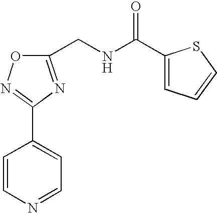

### N-((3-(pyridin-4-yl)-1,2,4-oxadiazol-5-yl)methyl)thiophene-2-carboxamide

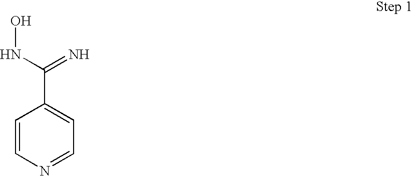

### N-hydroxyisonicotinimidamide

A 500 mL round bottom flask was charged with isonicotinonitrile (2.08 g, 20 mmol), hydroxyl amine (50% aqueous, 1.35 mL, 22 mmol), and ethanol (80 mL). The resulting mixture was heated in an 80° C. oil bath. Reaction progress was monitored by thin layer chromatography (50% ethylacetate/hexanes, Rf=0.05). After 2 h the reaction was complete. Work-up: concentrated to a powder, stored under high vacuum overnight, and used without further purification. Yield 2.74 g, 100% yield. ¹H NMR (400 MHz, DMSO-d₆) δ 10.04 (s, 1H), 8.56 (d, 2H), 7.63 (d, 2H), 6.00 (s, 2H).

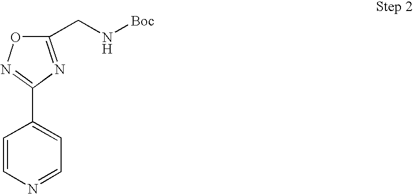

### tert-butyl (3-(pyridin-4-yl)-1,2,4-oxadiazol-5-yl)methylcarbamate

A 250 mL round bottom flask was charged with N-Boc-Glycine (1.92 g, 11.0 mmol), N-methyl morpholine (1.70 mL, 13.0 mmol), and THF (30 mL). To this solution stirring at −15° C. was added isobutyl choroformate (1.44 mL, 11.0 mmol). After 5 min at this temperature, N-hydroxyisonicotinimidamide (1.37 g, 10.0 mmol) dissolved in DMF (15 mL) was added. The resulting solution was warmed to room temperature over 30 min, then heated to 95° C. Reaction progress was monitored by HPLC, and was complete after stirring overnight. Work-up: the reaction concentrated, diluted with NaOH (50 mL, 1N aq.), extracted with ethylacetate, dried over MgSO₄, filtered, and concentrated giving a brown oil. The oil was purified by flash chromatography (40% to 50% ethylacetate/hexanes), giving 2.03 g of product (67% yield). LCMS (M+1)⁺: 277.8.

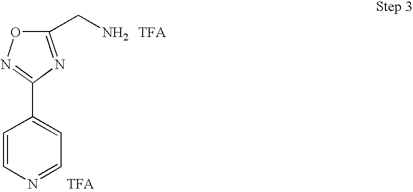

### (3-(pyridin-4-yl)-1,2,4-oxadiazol-5-yl)methanamine

A 100 mL round bottom flask was charged with tert-butyl (3-(pyridin-4-yl)-1,2,4-oxadiazol-5-yl)methylcarbamate (2.0 g, 7.24 mmol), and 30% TFA/methylene chloride (30 mL). The resulting solution was stirred at room temperature. Reaction progress was monitored by HPLC, and was complete after 45 min. Work-up: the reaction mixture was concentrated to brown oil (5.06g) that was used as is in subsequent steps. LCMS (M+1)⁺: 176.2.

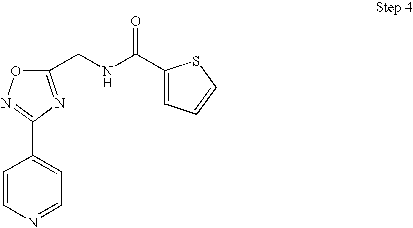

### N-((3-(pyridin-4-yl)-1,2,4-oxadiazol-5-yl)methyl)thiophene-2-carboxamide

A 10 mL round bottom flask was charged with (3-(pyridin-4-yl)-1,2,4-oxadiazol-5-yl)methanamine (349 mg of crude TFA salt, 0.5 mmol), methylene chloride (3.0 mL), triethylamine (418 μL, 3.0 mmol), followed by thiophene-2-carbonyl chloride (75 μL, 0.7 mmol). The resulting solution was stirred at room temperature for 5 min at which time HPLC indicated all starting amine had been consumed. Work-up: diluted with methylene chloride, extracted with NaOH (1N aq.), dried over MgSO₄, filtered, concentrated, and purified by C18 reverse phase semi-preparative HPLC, giving 77 mg (38% yield) of the title compound. ¹H NMR (400 MHz, DMSO-d₆) δ 9.46 (t, 1H), 8.82 (d, 2H), 7.97 (d, 2H), 7.83 (m, 2H), 7.18 (m, 1H), 4.83 (d, 2H). LCMS (M+1)⁺: 287.4.

### EXAMPLE 3

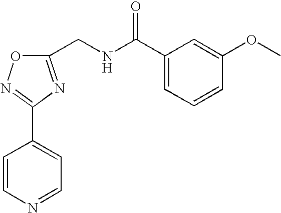

### 3-methoxy-N-((3-(pyridin-4-yl)-1,2,4-oxadiazol-5-yl)methyl)benzamide

The title compound was prepared analogously to N-((3-(pyridin-4-yl)-1,2,4-oxadiazol-5-yl)methyl)thiophene-2-carboxamide (Example 2), where 3-methoxy benzoylchloride was substituted for thiophene-2-carbonyl chloride in step 4 of that sequence. ¹H NMR (400 MHz, DMSO-d₆) δ 9.46 (t, 1H), 8.82 (d, 2H), 7.97 (d, 2H), 7.47 (m, 3H), 7.15 (m, 1H), 4.83 (d, 2H), 3.80 (s, 3H). LCMS (M+1)⁺: 311.8.

### EXAMPLE 4

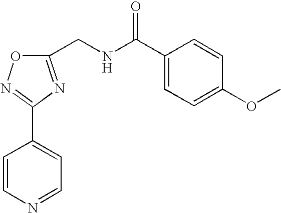

### 4-methoxy-N-((3-(pyridin-4-yl)-1,2,4-oxadiazol-5-yl)methyl)benzamide

The title compound was prepared analogously to N-((3-(pyridin-4-yl)-1,2,4-oxadiazol-5-yl)methyl)thiophene-2-carboxamide (Example 2), where 4-methoxy benzoylchloride was substituted for thiophene-2-carbonyl chloride in step 4 of that sequence. ¹H NMR (400 MHz, DMSO-d₆) δ 9.28 (t, 1H), 8.78 (d, 2H), 7.91 (d, 2H), 7.87 (d, 2H), 7.03 (d, 2H), 4.80 (d, 2H), 3.80 (s, 3H). LCMS (M+1)⁺: 311.7.

### EXAMPLE 5

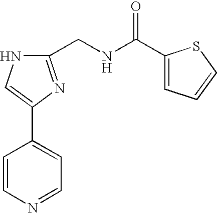

### N-((4-(pyridin-4-yl)-1H-imidazol-2-yl)methyl)thiophene-2-carboxamide

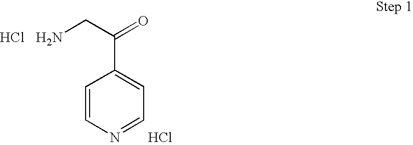

### 2-amino-1-(pyridin-4-yl)ethanone

A 100 mL round bottom flask was charged with 2,2-diethoxy-2-(pyridin-4-yl)ethanamine (2.38 g, 11.3 mmol, prepared as described in Org. Syn. Coll. Vol. 7 p. 149 (1990)), and concentrated HCl (15 mL). A precipitate formed immediately, which was heated in a 50° C. oil bath for 4 h. Work-up: the reaction mixture was concentrated to remove water, diluted with isopropanol forming a slurry that was sonicated, filtered, and further rinsed with isopropanol. The resulting orange solid was stored under high vacuum overnight, and used without further purification. Yield 1.82g, 77% yield. ¹H NMR (400 MHz, DMSO-d₆) δ 8.95 (d, 2H), 8.62 (bs, 4H), 8.06 (d, 2H), 4.65 (m, 2H).

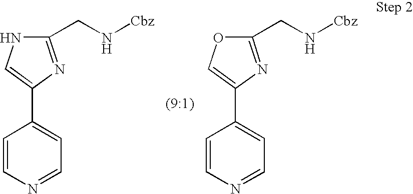

### Benzyl(4-(pyridin-4-yl)-1H-imidazol-2-yl)methylcarbamate and benzyl (4-(pyridin-4-yl)oxazol-2-yl)methylcarbamate

A 100 mL round bottom flask was charged with 2-amino-1-(pyridin-4-yl)ethanone (0.812 g, 4.0 mmol), methyl 2-(benzyloxycarbonylamino)acetimidate (1.78 g, 8.0 mmol, prepared as described in Bioorganic & Medicinal Chemistry (2006), 14(9), 3185-3198), KOAc (3.14g, 32.0 mmol), and methanol (40 mL). The resulting solution was heated in an oil bath at 65° C. Reaction progress was monitored by HPLC, which indicated all starting materials were consumed after stirring overnight. Work-up: the reaction was cooled, filtered to remove salts, concentrated onto silica, and purified by flash chromatography (gradient elution 5% to 10% methanol/methylene chloride). The resulting imidazole product was further recrystallized from methylene chloride and diethyl ether, yielding 243 mg (70%) LCMS (M+1)⁺: 309.4. The corresponding oxazole (benzyl (4-(pyridin-4-yl)oxazol-2-yl)methylcarbamate) was isolated as an faster eluting fraction during the column purification (27 mg, 7% yield). LCMS (M+1)⁺: 310.2.

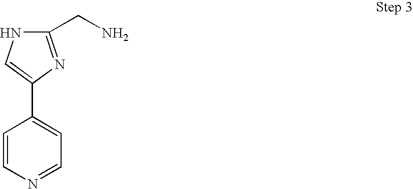

### (4-(pyridin-4-yl)-1H-imidazol-2-yl)methanamine

A 50 mL round bottom flask was charged with benzyl (4-(pyridin-4-yl)-1H-imidazol-2-yl)methylcarbamate (350 mg, 1.135 mmol), 10% Pd/C (350 mg), and methanol (10 mL). The resulting solution was vacuum purged with hydrogen from a balloon (3×) then stirred at room temperature for 1.5 h, at which time HPLC indicated all starting material had been consumed. Work-up: the mixture was filtered, concentrated, and used in subsequent steps without further purification. LCMS (M+1)⁺: 175.2.

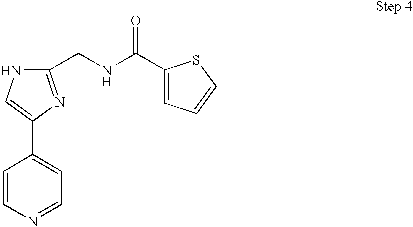

### N-((4-(pyridin-4-yl)-1H-imidazol-2-yl)methyl)thiophene-2-carboxamide

A 10 mL vial was charged with (4-(pyridin-4-yl)-1H-imidazol-2-yl)methanamine (97.4 mg, 0.41 mmol), thiophene-2-carboxylic acid (86.0 mg, 0.672 mmol), Et₃N (187 μL, 1.344 mmol), and HATU (255 mg, 0.672 mmol), in that order. The resulting mixture was stirred overnight at room temperature. The reaction progress was monitored by HPLC. Work-up: the reaction mixture purified directly by C18 reverse phase semi-preparative HPLC, giving the title compound 75 mg (49% yield). ¹H NMR (400 MHz, DMSO-d₆) δ 9.17 (t, 1H), 8.75 (d, 2H), 8.35 (s, 1H), 8.21 (d, 2H), 7.82 (m, 1H), 7.79 (m, 1H), 7.16 (m, 1H), 4.56 (d, 2H). LCMS (M+1)⁺: 284.9.

### EXAMPLE 6

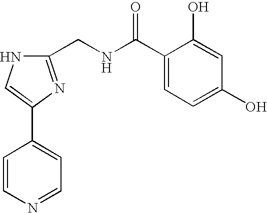

### 2,4-dihydroxy-N-((4-(pyridin-4-yl)-1H-imidazol-2-yl)methyl)benzamide

The title compound was prepared analogously to N-((4-(pyridin-4-yl)-1H-imidazol-2-yl)methyl)thiophene-2-carboxamide (Example 5), where 2,4-dihydroxybenzoic acid was substituted for thiophene-2-carboxylic acid in step 4 of that sequence. ¹H NMR (400 MHz, DMSO-d₆) δ 9.14 (t, 1H), 8.76 (m, 2H), 8.39 (s, 1H), 8.23 (m, 2H), 7.73 (d, 1H), 6.93 (d, 1H), 4.58 (d, 2H). LCMS (M+1)⁺: 311.0.

### EXAMPLE 7

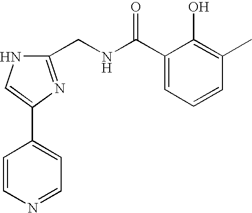

### 2-hydroxy-3-methyl-N-((4-(pyridin-4-yl)-1H-imidazol-2-yl)methyl)benzamide

The title compound was prepared analogously to N-((4-(pyridin-4-yl)-1H-imidazol-2-yl)methyl)thiophene-2-carboxamide (Example 5), where 2-hydroxy-3-methyl benzoic acid was substituted for thiophene-2-carboxylic acid in step 4 of that sequence. ¹H NMR (400 MHz, DMSO-d₆) δ 13.03 (s, 1H), 12.27 (bs, 1H), 9.45 (s, 1H), 8.46 (m, 2H), 7.81 (s, 1H), 7.75 (d, 1H), 7.67 (m, 2H), 7.31 (d, 1H), 6.78 (t, 1H), 4.55 (d, 2H), 2.15 (s, 3H). LCMS (M+1)⁺: 308.9.

### EXAMPLE 8

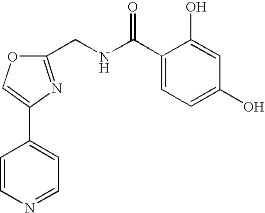

### 2,4-dihydroxy-N-((4-(pyridin-4-yl)oxazol-2-yl)methyl)benzamide

The title compound was prepared analogously to 2,4-dihydroxy-N-((4-(pyridin-4-yl)-1H-imidazol-2-yl)methyl)benzamide (Example 6), where (4-(pyridin-4-yl)oxazol-2-yl)methanamine (from step 2 of Example 5) was substituted for (4-(pyridin-4-yl)-1H-imidazol-2-yl)methanamine in step 4 of that sequence. ¹H NMR (400 MHz, DMSO-d₆) δ 12.37 (bs, 1H), 10.15 (bs, 1H), 9.22 (t, 1H), 8.77 (d, 2H), 8.10 (s, 1H), 7.85 (d, 2H), 7.73 (d, 1H), 6.32 (d, 1H), 6.25 (s, 1H), 4.67 (d, 2H). LCMS (M+1)⁺: 312.0.

### EXAMPLE 9

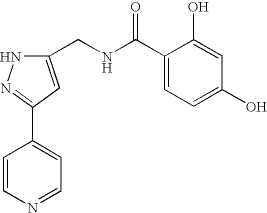

### 2,4-dihydroxy-N-((3-(pyridin-4-yl)-1H-pyrazol-5-yl)methyl)benzamide

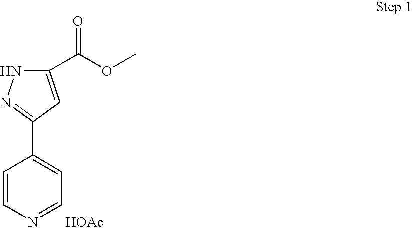

### Methyl 3-(pyridin-4-yl)-1H-pyrazole-5-carboxylate

A 500 mL round bottom flask cooled with an ice bath was charged with diisopropyl amine (5.11 mL, 60.0 mmol), THF (50 mL, anhydrous), diethyl ether (50 mL, anhydrous), and n-butyl lithium (22.0 mL, 55 mmol, 2.5M in hexanes) added dropwise, under nitrogen atmosphere. After 15 min at 0° C., the mixture was cooled to −78° C., where 1-(pyridin-4-yl)ethanone (5.50 g, 50 mmol), dissolved in THF (20 mL) was added dropwise over 15 min. The yellow enolate partially precipitates as it is formed. After 30 min at −78° C., dimethyl oxalate (5.9 g, 50 mmol) dissolved in THF (30 mL), was added dropwise over 15 min. After stirring for 15 min. at this temperature the cooling bath was removed for 10 min. during which time the reaction color changed from yellow to dark orange. The mixture was again cooled to −78° C., where diethyl ether (200 mL) was added in one portion causing formation of an orange precipitate. The precipitate/slurry was warmed to 0° C., filtered, and rinsed with diethyl ether. The resulting orange paste was quickly transferred to a 250 mL round bottom flask (decomposes rapidly on standing), dissolved in acetic acid (100 mL), and treated with hydrazine (3 mL, 95.5 mmol, neat). This solution was heated at 90° C. for 1.5 hr, under a nitrogen atmosphere. Reaction progress was monitored by TLC (50% acetonitrile/methylene chloride). Work-up: acetic acid was removed under vacuum. The resulting slurry was diluted with ethyl acetate/ether (1:1), and filtered. The filter cake was rinsed with ether, and dried under high vacuum overnight, resulting in 5.2 g, 40% yield of product as a white powder. ¹H NMR (400 MHz, DMSO-d₆) δ 8.61 (d, 2H), 7.83 (d, 2H), 7.51 (s, 1H), 3.85 (s, 3H), 1.89 (s, 3H).

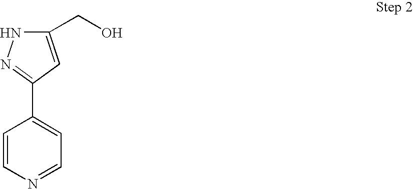

### (3-(pyridin-4-yl)-1H-pyrazol-5-yl)methanol

A 100 mL round bottom flask was charged with methyl 3-(pyridin-4-yl)-1H-pyrazole-5-carboxylate (1.053 g, 4.0 mmol), THF (36 mL, anhydrous), cooled to 0° C., then treated dropwise with lithium aluminum hydride (12 mL, 12.0 mmol, 1.0 M in ether). After 40 min LCMS showed no starting material. Work-up: the reaction was poured onto Na₂SO₄-10H₂O (˜200 g), diluted with 20% methanol/methylene chloride (100 mL), stirred for 5 min, and then decanted. This was repeated ten times. The combined organic layers were concentrated onto silica, and purified by flash chromatography (gradient elution, 2.5% to 10% methanol/methylene chloride). The combined column fractions were concentrated to slurry and filtered, giving 437 mg, 62% yield of product. ¹H NMR (400 MHz, DMSO-d₆) δ 8.55 (d, 2H), 7.72 (s, 1H), 6.76 (s, 1H), 5.3 (bs, 1H), 4.50 (s, 2H). LCMS (M+1)⁺: 176.14.

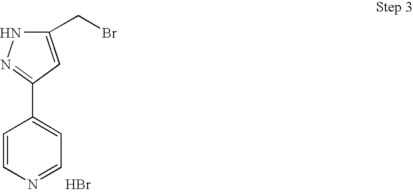

### 4-(5-(bromomethyl)-1H-pyrazol-3-yl)pyridine

A 35 mL sealed tube was charged with (3-(pyridin-4-yl)-1H-pyrazol-5-yl)methanol (200 mg, 1.14 mmol), and 33% HBr/HOAc (12 mL). The resulting flask was sealed with a teflon cap, and heated in a 75° C. oil bath for 7 hrs. Work-up: acetic acid and HBr were removed under vacuum, giving an oil. The oil was dissolved in methanol and acetonitrile, concentrated giving a slurry, which was filtered. The filter cake was rinsed with acetonitrile, and dried under high vacuum, giving the product as a yellow solid (295 mg, 81% yield). LCMS (M+1)⁺: 238.12.

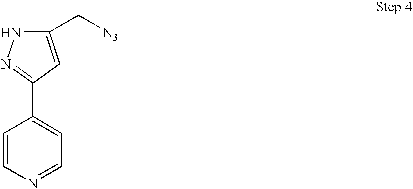

### 4-(5-(azidomethyl)-1H-pyrazol-3-yl)pyridine

A 100 mL round bottom flask was charged with 4-(5-(bromomethyl)-1H-pyrazol-3-yl)pyridine (1.2 g, 3.76 mmol), sodium azide (978 mg, 15.05 mmol), and DMF (10 mL). The resulting mixture was stirred at room temperature for 1 hr. Reaction progress was monitored by LCMS. Work-up: the reaction mixture was diluted with 15% methanol/methylene chloride (50 mL), extracted with NaHCO₃ (1N), dried with MgSO₄, filtered, and concentrated to a yellow oil (755 mg, quantitative yield), that was used without further purification. ¹H NMR (400 MHz, DMSO-d₆) δ 8.59 (bs, 2H), 7.74 (d, 2H), 6.94 (s, 1H), 4.48 (bs, 2H).

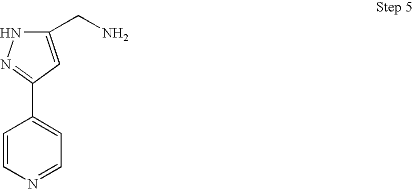

### (3-(pyridin-4-yl)-1H-pyrazol-5-yl)methanamine

A 100 mL round bottom flask was charged with 4-(5-(azidomethyl)-1H-pyrazol-3-yl)pyridine (750 mg, 3.74 mmol), 10% Pd/C (375 mg), and methanol (20 mL). The resulting mixture was vacuum flushed with hydrogen from a balloon (3×), and stirred at room temperature. Reaction progress was monitored by HPLC, which indicated all starting material had been consumed after 7 hrs. Work-up: the reaction mixture was filtered through a fine fritted filtered, and concentrated to an oil that was used without further purification (543 mg, 83% yield).

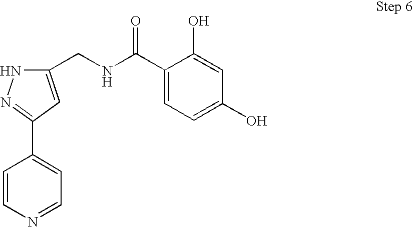

### 2,4-dihydroxy-N-((3-(pyridin-4-yl)-1H-pyrazol-5-yl)methyl)benzamide

A 10 mL vial was charged with (3-(pyridin-4-yl)-1H-pyrazol-5-yl)methanamine (130.6 mg, 0.75 mmol), 2,4-dihydroxybenzoic acid (116 mg, 0.75 mmol), HOBT (115 mg, 0.75 mmol), DMF (0.75 mL), THF (2.25 mL), followed by DCC (155 mg, 0.75 mmol). The resulting mixture was stirred for 1 hr at room temperature. Reaction progress was monitored by LCMS. Work-up: the reaction mixture was filtered and purified directly by C18 reverse phase semi-preparative HPLC, giving the title compound as a TFA salt, 174 mg (55% yield). ¹H NMR (400 MHz, DMSO-d₆) δ 12.64 (bs, 1H), 9.07 (t, 1H), 8.77 (d, 2H), 8.24 (d, 2H), 7.72 (d, 1H), 7.03 (s, 1H), 6.31 (d, 1H), 6.24 (s, 1H), 4.54 (d, 2H). LCMS (M+1)⁺: 310.83.

### EXAMPLE 10

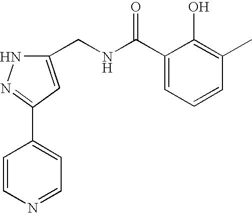

### 2-hydroxy-3-methyl-N-((3-(pyridin-4-yl)-1H-pyrazol-5-yl)methyl)benzamide

The title compound was prepared analogously to 2,4-dihydroxy-N-((3-(pyridin-4-yl)-1H-pyrazol-5-yl)methyl)benzamide (Example 9), where 2-hydroxy-3-methyl benzoic acid was substituted for 2,4-dihydroxybenzoic acid in step 6 of that sequence. ¹H NMR (400 MHz, DMSO-d₆) δ 13.01 (s, 1H), 9.45 (t, 1H), 8.75 (d, 2H), 8.16 (bs, 2H), 7.71 (d, 1H), 7.31 (s, 1H), 7.02 (s, 1H), 6.79 (t, 1H), 4.57 (d, 2H). LCMS (M+1)⁺: 310.18.

### EXAMPLE 11

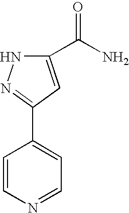

### 3-(pyridin-4-yl)-1H-pyrazole-5-carboxamide

A 20 mL microwave vial was charged with methyl 3-(pyridin-4-yl)-1H-pyrazole-5-carboxylate (527 mg, 2.0 mmol, described in step 1 of Example 9), and ammonium hydroxide (28% aq.10 mL). The resulting mixture was sealed and heated to 150° C. via microwave irradiation (Smith synthesizer) for 30 min. Reaction progress was monitored by TLC (75% acetonitrile/methylene chloride). Work-up: the reaction mixture was concentrated to remove most of the water, and then diluted with isopropanol which gave a precipitate. The precipitate was collected by filtration, rinsed with ether, and dried under high vacuum. The product was a white powder, 240 mg (64% yield). LCMS (M+1)⁺: 189.06.

### EXAMPLE 12

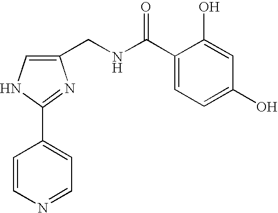

### 2,4-dihydroxy-N-((2-(pyridin-4-yl)-1H-imidazol-4-yl)methyl)benzamide

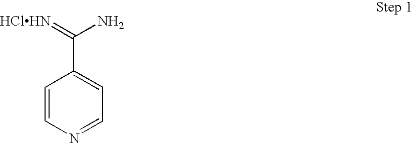

### Isonicotinimidamide hydrochloride

A 500 mL round bottom flask was charged with sodium metal (1.2 g, 52.17 mmol), and methanol (200 mL), and allowed to react until all sodium had dissolved. To this solution was added isonicotinonitrile (20.0 g, 192.31 mmol), which was stirred overnight at room temperature. The resulting imidate was treated with NH₄Cl (15.0 g, 280.4 mmol), and stirred for 4 hours at reflux. Reaction progress was monitored by TLC (EtOAc/petroleum ether=1:2). Work-up: the mixture was concentrated, diluted with 300 mL ethanol, warmed to reflux for 15 min., and then hot filtered. The filtrate was concentrated to a slurry, filtered, washed with ether, and dried under high vacuum. This gave 14 g (45% yield) of the product as a white solid. LCMS (M+1)⁺: 122.2.

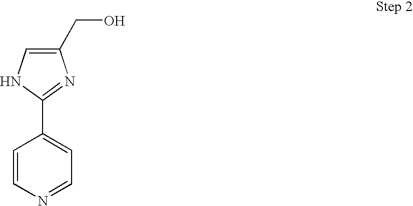

### (2-(pyridin-4-yl)-1H-imidazol-4-yl)methanol

A 100 mL round bottom flask was charged with isonicotinimidamide hydrochloride (2 g, 12.48 mmol), 1,3-dihydroxypropan-2-one (2.3 g, 25.56 mmol), and NH₄Cl (2.8 g, 52.34 mmol) in NH₃/H₂O (40 mL). The resulting solution was stirred for 30 min. in a 80° C. oil bath. Reaction progress was monitored by TLC (CH₂Cl₂/methanol=10:1). The resulting solution was extracted EtOAc (6×50 mL), dried over Na₂SO₄, concentrated, and triturated with ether forming a solid. The solid was isolated by filtration, dried under high vacuum, giving the product as a yellow solid 0.7g, 31%yield. ¹HNMR(300 MHz, DMSO-d₆) δ: 12.8 (d, 1H), 7.85 (d, 2H), 7.20 (s, 1H), 4.48 (s, 2H). LCMS (M+1)⁺: 176.4.

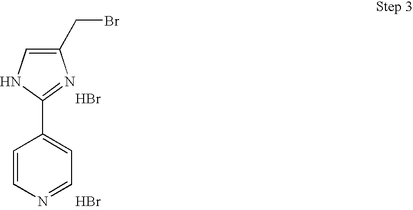

### 4-(4-(bromomethyl)-1H-imidazol-2-yl)pyridine dihydrobromide

A 500 mL round bottom flask was charged with (2-(pyridin-4-yl)-1H-imidazol-4-yl)methanol (15.0 g, 81.4 mmol), and 30% HBr/HOAc (300 mL). The resulting mixture was stirred for 4 hours at 80° C. Reaction progress was monitored by TLC (CH₂Cl₂/methanol=10:1). Work-up: the mixture was concentrated, forming a solid which was filtered, rinsed with ethanol (50 mL), and ether (100 mL). Drying under high vacuum gave the product as a yellow solid 28 g (86% yield), which was used without further purification.

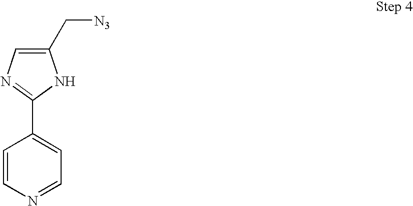

### 4-(5-(azidomethyl)-1H-imidazol-2-yl)pyridine

A 500 mL round bottom flask was charged with 4-(4-(bromomethyl)-1H-imidazol-2-yl)pyridine dihydrobromide (20.0 g, 59.9 mmol), DMF (60 mL), triethylamine (9.6 g, 95.1 mmol), and sodium azide (13.0 g, 200.0 mmol), while the temperature was maintained at −40° C. The resulting mixture was allowed to react overnight at room temperature. Reaction progress was monitored by TLC (CH₂Cl₂/methanol=10:1). Work-up: the reaction mixture was diluted with water/ice (200 mL), then extracted with EtOAc(4×200 mL), washed with water (1×100 mL), dried over Na₂SO₄, filtered, and concentrated to a solid (2.3 g, 18% yield) that was used without further purification.

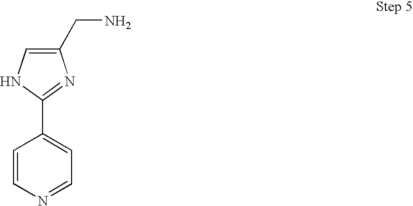

### (2-(pyridin-4-yl)-1H-imidazol-4-yl)methanamine

A 100 mL round bottom flask was charged with of SnCl₂-2H₂O (8.5 g, 37.61 mmol), and methanol (30 mL), to which a solution of 4-(5-(azidomethyl)-1H-imidazol-2-yl)pyridine (2.5 g, 12.25 mmol) dissolved in methanol (10 mL) was added dropwise. The mixture was stirred overnight at room temperature. Reaction progress was monitored by TLC (CH₂Cl₂/methanol=10:1). Work-up: the mixture was concentrated, dissolved in water (20 mL), pH adjusted to 12 with NaOH (3M), extracted with ethyl acetate (4×50 mL), dried over Na₂SO₄, and concentrated to an oil that was purified by flash chromatography (5:1 THF/methanol). This resulted in 1.15 g (54% yield) of product as a yellow solid. ¹H NMR (300 MHz, DMSO-d₆) δ: 8.62 (d, 2H), 7.9 (d, 2H), 7.36 (s, 1H), 3.9 (s, 2H). LCMS (M+1)⁺: 175.3.

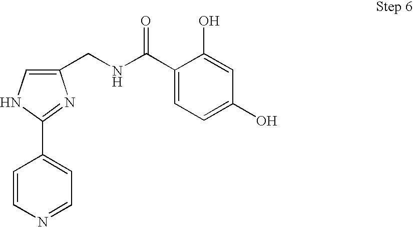

### 2,4-dihydroxy-N-((2-(pyridin-4-yl)-1H-imidazol-4-yl)methyl)benzamide

A 5 mL vial was charged with (2-(pyridin-4-yl)-1H-imidazol-4-yl)methanamine (130.6 mg, 0.75 mmol), 2,4-dihydroxybenzoic acid (116 mg, 0.75 mmol), DMF (0.75 mL), THF (3.0 mL), followed by DCC (155 mg, 0.75 mmol). The resulting mixture was stirred overnight at room temperature. Reaction progress was monitored by LCMS. Work-up: the reaction mixture was filtered and purified directly by C18 reverse phase semi-preparative HPLC, giving the title compound as a bis-TFA salt, 51 mg (13% yield). ¹H NMR (400 MHz, DMSO-d₆) δ: 9.00 (t, 1H), 8.81 (d, 2H), 8.15 (d, 2H), 7.72 (d, 2H), 6.28 (d, 1H), 6.23 (s, 1H), 4.50 (d, 2H). LCMS (M+1)⁺: 311.01.

### EXAMPLE 13

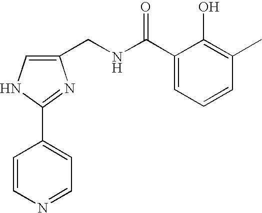

### 2-hydroxy-3-methyl-N-((2-(pyridin-4-yl)-1H-imidazol-4-yl)methyl)benzamide

The title compound was prepared analogously to 2,4-dihydroxy-N-((2-(pyridin-4-yl)-1H-imidazol-4-yl)methyl)benzamide (Example 12), where 2-hydroxy-3-methyl benzoic acid was substituted for 2,4-dihydroxybenzoic acid in step 6 of that sequence. ¹H NMR (400 MHz, DMSO-d₆) δ: 13.24 (bs, 1H), 12.83 (bs, 1H), 9.33 (bs, 1H), 8.59 (d, 2H), 7.82 (d, 2H), 7.75 (d, 1H), 7.29 (d, 1H), 7.25 (s, 1H), 6.75 (t, 1H), 4.45 (d, 2H), 2.14 (s, 3H). LCMS (M+1)⁺: 309.8.

### EXAMPLE 14

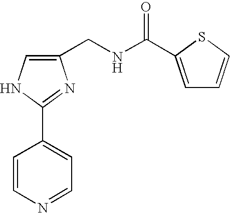

### 5-methyl-N-((2-(pyridin-4-yl)-1H-imidazol-4-yl)methyl)thiophene-2-carboxamide

The title compound was prepared analogously to 2,4-dihydroxy-N-((2-(pyridin-4-yl)-1H-imidazol-4-yl)methyl)benzamide (Example 12), where thiophene-2-carboxylic acid was substituted for 2,4-dihydroxybenzoic acid in step 6 of that sequence. ¹H NMR (400 MHz, DMSO-d₆) δ: 12.84 (bs, 1H), 8.92 (t, 1H), 8.59 (d, 2H), 7.82 (d, 2H), 7.72 (d, 1H), 7.18 (bs, 1H), 7.11 (t, 1H), 4.40 (bs, 2H). LCMS (M+1)⁺: 284.97.

### EXAMPLE 15

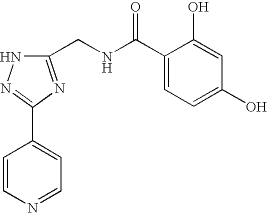

### 2, 4-dihydroxy-N-((3-(pyridin-4-yl)-1H-1,2,4-triazol-5-yl) methyl)benzamide

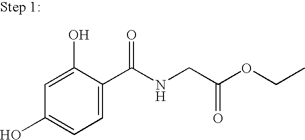

### Ethyl 2-(2,4-dihydroxybenzamido)acetate

A 500 mL round bottom flask was charged with ethyl 2-aminoacetate hydrochloride (6.142 g, 44.0 mmol), 2,4-dihydroxybenzoic acid (6.165 g, 40.0 mmol), 4-methylmorpholine (4.83 mL, 130 mmol), HOBT (673 mg, 4.40 mmol), THF (160 mL), and diisopropyl carbodiimide (6.81 mL, 44.0 mmol). The solution was stirred for 3 hours at room temperature, and progress was monitored by TLC (EtOAc/Hex=3:10). Workup: the reaction was diluted with methylene chloride (200 mL); washed with saturated sodium bicarbonate solution, 1N HCl, and brine. The organics were dried over MgSO₄, and the urea was filtered off. The remaining filtrate was concentrated onto silica, and was chromatographed (EtOAc/Hex=30-50% via step gradient), to give 6.95 g of a white solid product (73% yield).

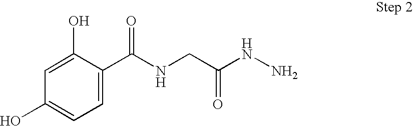

### N-(2-hydrazinyl-2-oxoethyl)-2, 4-dihydroxybenzamide

A 200 mL round bottom flask was charged with ethyl 2-(2,4-dihydroxybenzamido)acetate (5.81 g, 24.3 mmol), methanol (25 mL), and hydrazine (3.8 mL, 122.0 mmol). The reaction was stirred for 2.5 hours at 60° C. Workup: the reaction was cooled to room temperature; and the solid precipitate was filtered; washed with methanol; and dried to give 5.25 g of product.

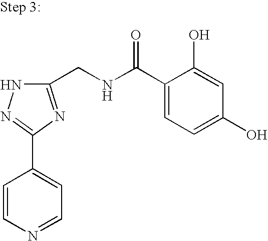

### 2,4-dihydroxy-N-((3-(pyridin-4-yl)-1H-1,2,4-triazol-5-yl) methyl) benzamide

A 300 mL sealed tube was charged with N-(2-hydrazinyl-2-oxoethyl)-2,4-dihydroxybenzamide (3.378 g, 15.0 mmol), isonicotinonitrile (4.68 g, 45.0 mmol), DBU (2.24 mL, 15.0 mmol), and n-butanol (45 mL). The suspension was stirred at 140° C. for 4 hours in a sealed tube. Reaction progress was monitored by the disappearance of hydrazide on TLC. Workup: the mixture was cooled to room temperature; concentrated to remove n-butanol; and was recrystallized in MeOH/methylene chloride to give 4.15 g of a light yellow powder, as product (67% yield). ¹H NMR (400 MHz, DMSO-d₆): δ 12.50 (bs, 1H), 10.1 1 (bs, 1H), 9.19 (bs, 1H), 8.65 (m, 1H), 7.88 (dd, 2H), 7.72 (d, 1H), 6.30 (dd, 1H), 6.24 (d, 1H), 4.74 (bs, 2H), 2.71 (s, 1H). LCMS (M+1)⁺: 311.87.

### EXAMPLE 16

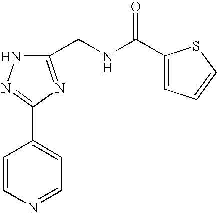

### 1-(2, 6-dichlorophenyl)-3-methyl-5-(pyridin-2-ylmethoxy)-1H-imidazo[4,5-b]pyridin-2 (3H)-one

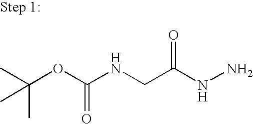

### tert-butyl 2-hydrazinyl-2-oxoethylcarbamate

A 1 liter round bottom flask was charged with 2-(tert-butoxycarbonylamino) acetic acid (17.5 g, 100 mmol), THF (400 mL), 4-methylmorpholine (14.30 mL, 130 mmol), and ethyl chloroformate (10.5 mL, 110 mmol). The solution was stirred for 2 hours, where a white solid precipitated. The precipitate was filtered off; and the remaining filtrate was added slowly to a cooled solution of hydrazine (15.7 mL, 500 mmol). The reaction was initially stirred at 0° C., and was allowed to wart to room temperature over the course of 1 hour. Reaction progress was monitored by TLC (MeOH/methylene chloride=1:0). Workup: the reaction was quenched with water (150 mL), and extracted into EtOAc (200 mL). The organic layer was then washed with 1M citric acid, saturated sodium bicarbonate, and brine. The organics were dried over MgSO₄; and concentrated to give 17.3 g of product as a white solid (92% yield).

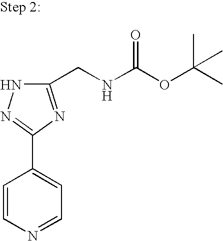

### tert-butyl(3-(pyridin-4-yl)-1H-1,2,4-triazol-5-yl)methylcarbamate

A 300 mL, sealed tube was charged with tert-butyl 2-hydrazinyl-2-oxoethylcarbamate (6.7 g, 30 mmol), isonicotinonitrile (9.37 g, 90 mmol), K₂CO₃ (1.24 g, 9 mmol), and butan-1-ol (60 mL). The suspension was heated to 150° C., and was allowed to stir overnight. The suspension dissolved at 100° C. The reaction progress was monitored by the disappearance of hydrazide on TLC (MeOH/methylene chloride=1:20). Workup: the mixture was cooled to room temperature; K₂CO₃ removed by filtration, and the filtrate concentrated, and chromatographed on 330 g of silica gel (2 to 8% MeOH/methylene chloride, gradient elution), to give 6 g of a white solid product (55% yield). LCMS (M+1)⁺: 276.88 (M+1)⁺.

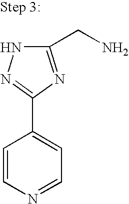

### (3-(pyridin-4-yl)-1H-1,2,4-triazol-5-yl)methanamine

A 100 mL flask was charged with tert-butyl (3-(pyridin-4-yl)-1H-1,2,4-triazol-5-yl) methylcarbamate (2.4 g, 8.7 mmol), dichloromethane (32 mL), and trifluoroacetic acid (16 mL). The reaction was stirred at room temperature, and was completed in 40 minutes. The reaction was monitored by TLC (MeOH/methylene chloride=1:10). Workup: the reaction mixture was diluted with toluene (10 mL), and concentrated to give 3.78 g of product as a bis-TFA salt. ¹H NMR (400 MHz, DMSO-d₆): δ 8.66 (d, 2H), 7.89 (d, 2H), 3.99 (s, 2H), 3.30 (bs, 2H). LCMS (M+1)⁺: 175.97 (M+1)⁺.

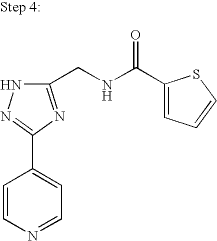

### N-((3-(pyridin-4-yl)-1H-1,2,4-triazol-5-yl) methyl)thiophene-2-carboxamide

A 20 mL vial was charged with the TFA salt of (3-(pyridin-4-yl)-1H-1,2,4-triazol-5-yl) methanamine (520 mg, 1.28 mmol), thiophene-2-carbonyl chloride (137 μl, 1.28 mmol), triethylamine (0.9 mL, 6.40 mmol), and methylene chloride (4.27 mL). The reaction was stirred at room temperature for 5 minutes. Reaction progress was monitored by LC/MS. Workup: the resulting mixture gave bis-acylated product which hydrolyzed to the mono-acyl product by treating with sodium hydroxide (0.5 mL 1N), and methanol (1 mL). The resulting solution was concentrated, dissolved in DMSO, and purified via reverse phase HPLC, giving 300 mg of product as a TFA salt (82% yield). ¹H NMR (400 MHz, DMSO-d₆): δ 9.25 (bs, 1H), 8.80 (d, 2H), 8.17 (d, 2H), 7.16 (dd, 2H), 7.16 (t, 1H), 4.62 (d, 3H). LCMS (M+1)⁺: 285.90(M+1)⁺.

### EXAMPLE 17

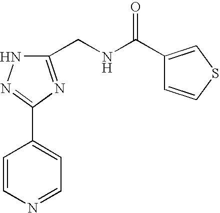

### N-((3-(pyridin-4-yl)-1H-1,2,4-triazol-5-yl)methyl)thiophene-3-carboxamide

The title compound was prepared analogously to Example 16, where thiophene-3-carbonyl chloride was substituted for thiophene-2-carbonyl chloride in step 4 of that sequence. ¹H NMR (400 MHz, DMSO-d₆): δ 9.03 (bs, 1H), 8.75 (d, 2H), 8.20 (d, 2H), 8.07 (bs, 1H), 7.6 (q, 1H), 7.56(q, 1H), 4.61 (d, 2H). LCMS (M+1)⁺: 286.42.

### EXAMPLE 18

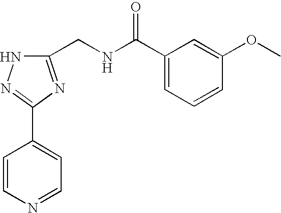

### 3-methoxy-N-((3-(pyridin-4-yl)-1H-1,2,4-triazol-5-yl) methyl)benzamide

The title compound was prepared analogously to Example 16, where 3-methoxybenzoyl chloride was substituted for thiophene-2-carbonyl chloride in step 4. ¹H NMR (400 MHz, CD₃OD): δ 8.80 (d, 2H), 8.42 (d, 2H), 7.47 (s, 1H), 7.37 (t, 2H), 7.12, (d, 1H), 4.79 (d, 2H), 3.84 (s, 3H). LCMS (M+1)⁺: 310.66.

### EXAMPLE 19

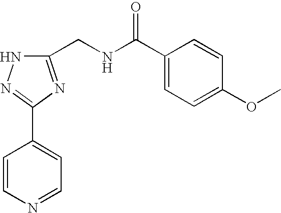

### 4-methoxy-N-((3-(pyridin-4-yl)-1H-1,2,4-triazol-5-yl) methyl)benzamide

An 8 mL vial was charged with the TFA salt of (3-(pyridin-4-yl)-1H-1,2,4-triazol-5-yl) methanamine (81.2 mg, 0.20 mmol), DMF (2 mL), 4-methoxybenzoic acid (36.5 mg, 0.24 mmol), triethylamine (112 μl, 0.80 mmol), and O-(7-Azabenzotriazol-1-yl)-1,1,3,3-tetramethyluronium hexafluorophosphate (HATU) (91.3 mg, 0.24 mmol). The reaction was stirred at room temperature for 5 minutes. Reaction progress was monitored by LC/MS. Workup: the reaction was neutralized, diluted with DMSO, and purified via reverse phase HPLC; giving 62 mg of a crystalline product as a TFA salt (73% yield).

¹H NMR (400 MHz, DMSO-d₆): δ 9.06 (bs, 1H), 8.80 (d, 2H), 8.17 (bs, 1H), 7.89 (d, 2H), 7.01 (d, 2H), 4.63 (d, 2H), 3.80 (s, 3H). LCMS (M+1)⁺: 310.09(M+1)⁺

### EXAMPLE 20

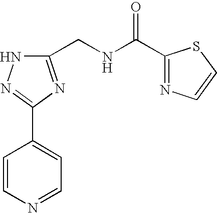

### N-((3-(pyridin-4-yl)-1H-1,2,4-triazol-5-yl) methyl) thiazole-2-carboxamide

The title compound was prepared analogously to Example 19, where thiazole-2-carboxylic acid was substituted for 4-methoxybenzoic acid in step 4 (34% yield). ¹H NMR (400 MHz, CD₃OD): δ 8.73 (d, 2H), 8.40 (d, 2H), 7.90 (s, 1H), 7.79 (s, 1H), 4.74 (s, 2H), 2.71 (s, 1H). LCMS (M+1)⁺: 286.83.

### EXAMPLE 21

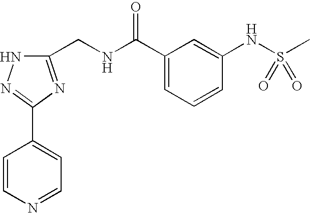

### 3-(methylsulfonamido)-N-((3-(pyridin-4-yl)-1H-1,2,4-triazol-5-yl)methyl)benzamide

The title compound was prepared analogously to Example 19, where 3-(methylsulfonamido)benzoic acid was substituted for 4-methoxybenzoic acid in step 4 (77% yield). ¹H NMR (400 MHz, DMSO-d₆): δ 9.91 (s, 1H), 9.20 (bs, 1H), 8.73 (m, 1H), 8.05 (bs, 1H), 7.71 (d, 1H), 7.64 (dd, 1H), 7.44 (t, 1H), 7.37 (m, 1H), 4.64 (d, 2H), 2.67 (s, 3H). LCMS (M+1)⁺: 372.68.

### EXAMPLE 22

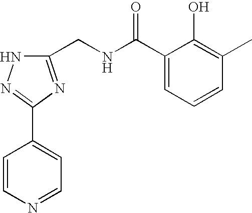

### 2-hydroxy-3-methyl-N-((3-(pyridin-4-yl)-1H-1,2,4-triazol-5-yl)methyl)benzamide

The title compound was prepared analogously to Example 19, where 2-hydroxy-3-methylbenzoic acid was substituted for 4-methoxybenzoic acid in step 4 (32% yield). ¹H NMR (400 MHz, CD₃OD): δ 8.80 (d, 2H), 8.45 (d, 2H), 7.59 (d, 1H), 7.29 (d, 1H), 6.81 (t, 1H), 4.79 (s, 2H), 2.21 (s, 3H). LCMS (M+1)⁺: 309.74.

### EXAMPLE 23

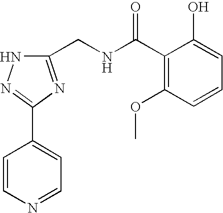

### 2-hydroxy-6-methoxy-N-((3-(pyridin-4-yl)-1H-1,2,4-triazol-5-yl)methyl)benzamide

The title compound was prepared analogously to Example 19, where 2-hydroxy-6-methoxybenzoic acid was substituted for 4-methoxybenzoic acid in step 4 (16% yield). ¹H NMR (400 MHz, DMSO-d₆): δ 9.36 (t, 1H), 8.82 (d, 2H), 8.21 (d, 2H), 7.35 (t, 1H), 6.60 (d, 1H), 6.50 (d, 1H), 4.74 (d, 2H), 3.93 (s, 3H). LCMS (M+1)⁺: 325.80.

### EXAMPLE 24

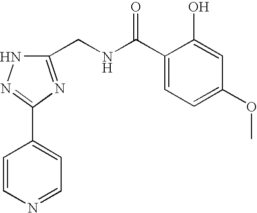

### 2-hydroxy-4-methoxy-N-((3-(pyridin-4-yl)-1H-1,2,4-triazol-5-yl)methyl)benzamide

The title compound was prepared analogously to Example 19, where 2-hydroxy-4-methoxybenzoic acid was substituted for 4-methoxybenzoic acid in step 4 (32% yield). ¹H NMR (400 MHz, DMSO-d₆): δ 12.63 (bs,1H), 9.38 (bs, 1H), 8.64 (d, 2H), 7.89 (dd, 2H), 7.83 (d, 1H), 6.49 (dd, 1H), 6.41 (d, 1H), 4.64( ), 2.71 (s, 1H). LCMS (M+1)⁺: 325.78.

### EXAMPLE 25

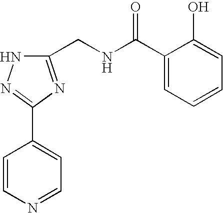

### 2-hydroxy-N-((3-(pyridin-4-yl)-1H-1,2,4-triazol-5-yl)methyl)benzamide

The title compound was prepared analogously to Example 19, where 2-hydroxy benzoic acid was substituted for 4-methoxybenzoic acid in step 4 (32% yield). ¹H NMR (400 MHz, DMSO-d₆): δ 12.09 (bs, 1H), 9.45 (m, 1H), 8.78 (d, 2H), 8.17 (d, 1H), 7.89 (dd, 1H), 7.40 (t, 1H), 6.89 (t, 2H), 4.68 (d, 2H). LCMS (M+1)⁺: 296.01(M+1)

### EXAMPLE 26

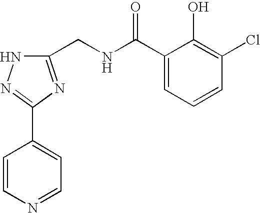

### 3-chloro-2-hydroxy-N-((3-(pyridin-4-yl)-1H-1,2,4-triazol-5-yl)methyl)benzamide

The title compound was prepared analogously to Example 19, where 3-chloro-2-hydroxybenzoic acid was substituted for 4-methoxybenzoic acid (32% yield). ¹H NMR (400 MHz, DMSO-d₆): 6 13.25 (bs,1H), 9.77 (bs, 1H), 8.78 (d, 2H), 8.12 (m, 2H), 7.89 (dd, 1H), 7.63 (dd, 1H), 6.95 (t, 1H), 4.65 (d, 2H). LCMS (M+1)⁺: 330.03.

### EXAMPLE 27

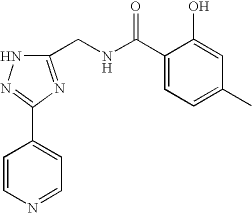

### 2-hydroxy-4-methyl-N-((3-(pyridin-4-yl)-1H-1,2,4-triazol-5-yl)methyl) benzamide

The title compound was prepared analogously to Example 19, where 2-hydroxy-4-methylbenzoic acid was substituted for 4-methoxybenzoic acid (17% yield). ¹H NMR (400 MHz, DMSO-d₆): 6 12.23 (bs,1H), 9.38 (bs, 1H), 8.74 (d, 2H), 8.05 (m, 2H), 7.78 (d, 1H), 6.73 (m, 1H), 4.66 (d, 2H), 2.26 (s, 3H). LCMS (M+1)⁺: 310.02.

### EXAMPLE 28

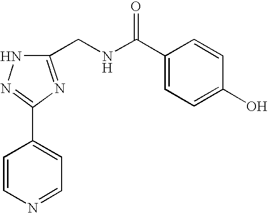

### 4-hydroxy-N-((3-(pyridin-4-yl)-1H-1,2,4-triazol-5-yl)methyl)benzamide

The title compound was prepared analogously to Example 19, where 4-hydroxybenzoic acid was substituted for 4-methoxybenzoic acid (2% yield). ¹H NMR (400 MHz, CD₃OD): δ 8.80 (d, 2H), 8.18 (d, 2H), 7.78 (d, 2H), 6.80 (d, 2H), 4.60 (d, 2H). LCMS (M+1)⁺: 295.96.

### EXAMPLE 29

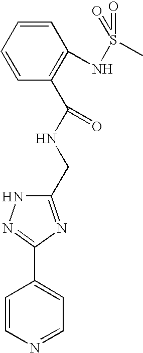

### 2-(Methylsulfonamido)-N-((3-(pyridin-4-yl)-1H-1,2,4-triazol-5-yl)methyl)benzamide

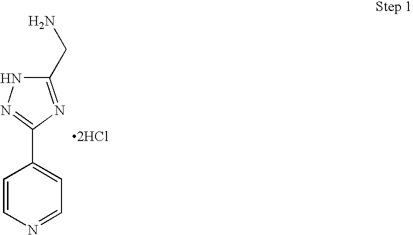

### (3-(Pyridin-4-yl)-1H-1,2,4-triazol-5-yl)methanamine, hydrochloride

A 50 mL round bottom flask was charged with tert-butyl (3-(pyridin-4-yl)-1H-1,2,4-triazol-5-yl)methylcarbamate (138 mg, 0.501 mmol, prepared as described in Example 16), and HCl in dioxane (4.0 M, 5 mL, 20 mmol), then stirred in an oil bath at 90° C. for 75 min. Reaction progress was monitored by LCMS. Work-up: the mixture was allowed to cool and then concentrated to dryness and used in the next step without further purification.

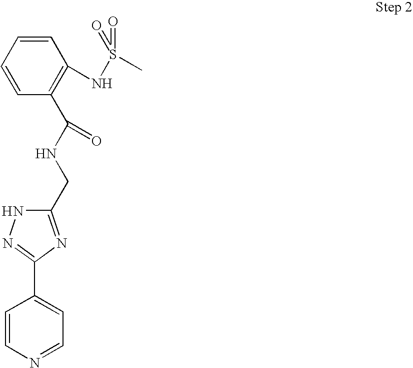

### 2-(Methylsulfonamido)-N-((3-(pyridin-4-yl)-1H-1,2,4-triazol-5-yl)methyl)benzamide

A 25 mL round bottom flask was charged with crude (3-(pyridin-4-yl)-1H-1,2,4-triazol-5-yl)methanamine, hydrochloride (0.501 mmol theoretical yield), 2-(methylsulfonamido)benzoic acid (108 mg, 0.502 mmol, prepared as described in Eur. J. Med. Chem. 2002, 37, 147), N,N′-dicyclohexycarbodiimide (103 mg, 0.499 mmol), 1-hydroxybenzotriazole hydrate (4 mg, 0.03 mmol), triethylamine (0.279 mL, 2.00 mmol) and a 4:1 mixture of THF and DMF (5 mL) The resulting mixture was stirred overnight at room temperature, then filtered and evaporated. The crude product was purified by C18 reverse phase semi-preparative HPLC, giving the product as a faintly yellow thick oil (mono TFA salt, 159 mg, 65% for two steps). ¹H NMR (400 MHz, DMSO-d₆) δ: 10.79 (bs, 1H), 8.74 (m, 2H), 8.52 (m, 2H), 8.09 (bm, 1H), 7.78 (m, 1H), 7.66 (m, 1H), 7.58 (m, 1H), 7.24 (m, 1H), 4.76 (d, 2H), 3.02 (s, 3H). LCMS (M+1⁻): 373.14.

### EXAMPLE 30

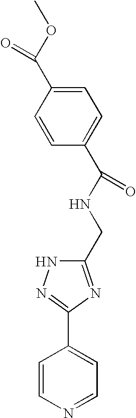

### Methyl 4-((3-(pyridin-4-yl)-1H-1,2,4-triazol-5-yl)methylcarbamoyl)benzoate

To a solution of (3-(pyridin-4-yl)-1H-1,2,4-triazol-5-yl)methanamine (0.25g, 1 mmol, described in Example 16) in DMF (5 mL), was added 4-(methoxycarbonyl)benzoic acid (0.18 g, 0.24 mmol), triethylamine (0.5 g, 5 mmol), followed by HATU (0.42 g, 1.1 mmol). The resulting solution was stirred at room temperature overnight. Reaction progress was monitored by LCMS. The reaction was diluted with water, extracted with ethyl acetate (3×50 mL), water, brine, dried over Na₂SO₄, filtered, and concentrated. The crude product was purified by C18 reversed phase HPLC, giving the title compound (0.21 g, 62% yield) as an off-white solid. ¹H NMR (400 MHz, DMSO-d6): δ 9.40 (s, 1H), 8.74 (d, 2H), 8.07-8.01 (m, 6H), 4.66 (d, 2H), 3.87 (s, 3H).

### EXAMPLE 31

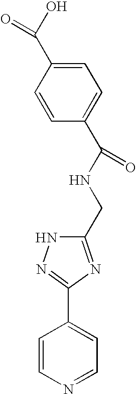

### 4-((3-(Pyridin-4-yl)-1H-1,2,4-triazol-5-yl)methylcarbamoyl)benzoic acid

To a solution of methyl 4-((3-(pyridin-4-yl)-1H-1,2,4-triazol-5-yl) methylcarbamoyl)benzoate (0.1 g, 0.3 mmol, Example 30) in THF (1.5 mL), was added LiOH (0.008 g, 0.33 mmol, in minimum amount of water). The mixture was stirred at room temperature overnight. Work-up: the reaction was concentrated, dissolved in DMSO, and purified by preparative C18 reversed phase HPLC, eluted giving the title compound (0.080 g, 85% yield) as an off-white solid. ¹H NMR (400 MHz, DMSO-d6): δ 9.40 (t, 1H), 8.73-8.71 (m, 2H), 8.04-7.99 (m, 6H), 4.66 (d, 2H), 3.87 (s, 3); LCMS: (M+1)⁺: 324.02.

### EXAMPLE 32

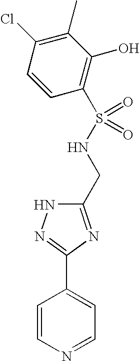

### 4-Chloro-2-hydroxy-3-methyl-N-((3-(pyridin-4-yl)-1H-1,2,4-triazol-5-yl)methyl)benzenesulfonamide

To a solution of (3-(pyridin-4-yl)-1H-1,2,4-triazol-5-yl)methanamine (0.05 g, 0.15 mmol, described in Example 16), and 4-chloro-2-hydroxy-3-methylbenzene-1-sulfonyl chloride (0.036 g, 0.15 mmol) in acetonitrile (1.5 mL), was added K₂CO₃ (0.041 g, 0.3 mmol). The mixture was stirred at 70° C. overnight. Work-up: the reaction was cooled, filtered, concentrated in vacuo, and purified by C18 reversed phase HPLC, giving the title compound (0.015 g, 27% yield) as an off-white solid. LCMS: (M+1)⁺: 389.88.

### EXAMPLE 33

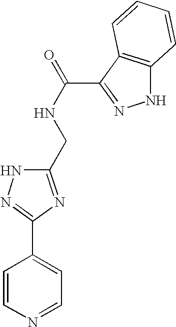

### N-((3-(Pyridin-4-yl)-1H-1,2,4-triazol-5-yl)methyl)-1H-indazole-3-carboxamide

The title compound was prepared analogously to methyl 4-((3-(pyridin-4-yl)-1H-1,2,4-triazol-5-yl)methylcarbamoyl)benzoate (Example 30), where 1H-indazole-3-carboxylic acid was substituted for 4-(methoxycarbonyl)benzoic acid. ¹H NMR (400 MHz, CD₃OD): δ 8.82 (d, 2H), 8.51 (d, 2H), 8.21 (d, 1H), 7.59 (d, 1H), 7.42 (t, 1H), 7.26 (t, 1H), 4.91-4.82 (m, 1H); LCMS: (M+1)⁺: 319.98.

### EXAMPLE 34

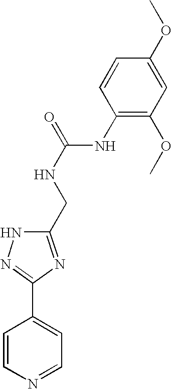

### 1-(2,4-Dimethoxyphenyl)-3-((3-(pyridin-4-yl)-1H-1,2,4-triazol-5-yl)methyl)urea

To a suspension of (3-(pyridin-4-yl)-1H-1,2,4-triazol-5-yl)methanamine (0.025 g, 0.074 mmol, described in Example 16) in methylene chloride (0.74 mL) was added 1-isocyanato-2,4-dimethoxybenzene (0.013 g, 0.074 mmol), followed by triethyl amine (3 eq.). The mixture was allowed to stir for overnight at room temperature. Reaction progress was monitored by LCMS. Work-up: the reaction was concentrated and purified by C18 reversed phase HPLC, giving the title compound (0.012 g, 46% yield) as an off-white solid. ¹H NMR (400 MHz, DMSO-d₆): δ 8.73 (d, 2H), 8.05 (s, br, 2H), 7.90-7.83 (m, 3H), 7.31 (s, 1H), 6.56 (d, 1H), 6.41 (dd, 1H), 4.45 (d, 2H), 3.80 (s, 3H), 3.69 (s, 3H); LCMS: (M+1)⁺: 355.03.

### EXAMPLE 35

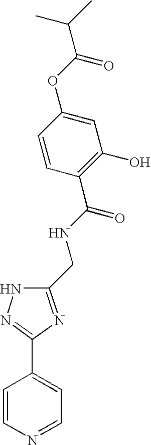

### 3-Hydroxy-4-((3-(pyridin-4-yl)-1H-1,2,4-triazol-5-yl)methylcarbamoyl)phenyl isobutyrate

To a solution of 2,4-dihydroxy-N-((3-(pyridin-4-yl)-1H-1,2,4-triazol-5-yl)methyl)benzamide (0.2 g, 0.63 mmol, from Example 15) in pyridine (6.4 mL), was added isobutyryl chloride (0.27 g, 2.57 mmol) dropwise at ° C. The resulting mixture was stirred at this temperature for 1 h. Reaction progress was monitored by LCMS. Work-up: the reaction was concentrated, diluted with ethyl acetate (100 mL), extracted with ethyl acetate (2×200 mL), brine, dried over Na₂SO₄, filtered, concentrated. The resulting di-acylated material was dissolved in MeOH (63 mL) stirred overnight at room temperature, then heated to complete conversion to the desired mono-acyl product. Work-up: the mixture concentrated and purified by silica gel chromatography eluted with 0-10% MeOH and methylene chloride, giving the title compound (0.18g, 75% yield) as an off-white solid. ¹H NMR (400 MHz, DMSO-d₆): δ 12.48 (s, 1H), 9.44 (s, 1H), 8.65 (br, 2H), 7.96-7.88 (m, 3H), 6.71-6.69 (m, 2H), 4.67 (d, 2H), 2.83-2.76 (m, 1H), 1.21 (d, 6H); LCMS: (M+1)⁺: 383.16.

### EXAMPLE 36

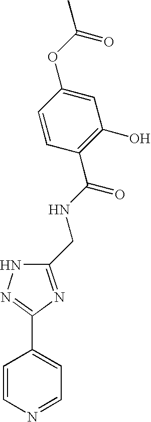

### 3-Hydroxy-4-((3-(pyridin-4-yl)-1H-1,2,4-triazol-5-yl)methylcarbamoyl)phenyl acetate

The title compound was prepared analogously to 3-hydroxy-4-((3-(pyridin-4-yl)-1H-1,2,4-triazol-5-yl)methylcarbamoyl)phenyl isobutyrate (Example 35), where acetyl chloride was substituted for isobutyryl chloride. ¹H NMR (400 MHz, DMSO-d₆): δ 12.48 (s, 1H), 9.45 (s, 1H), 8.65 (d, 2H), 7.95-7.88 (m, 3H), 6.71-6.69 (m, 2H), 4.67 (d, 2H), 2.25(s, 3H); LCMS: (M+1)⁺: 353.99.

### EXAMPLE 37

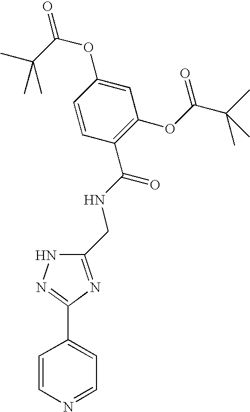

### 4-((3-(Pyridin-4-yl)-1H-1,2,4-triazol-5-yl)methylcarbamoyl)-1,3-phenylene bis(2,2-dimethylpropanoate):

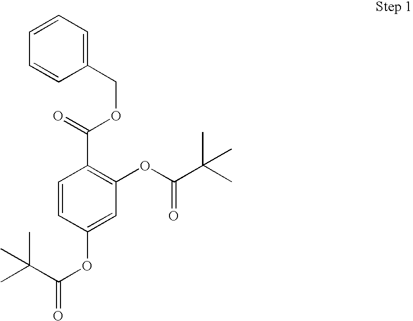

### 4-(Benzyloxycarbonyl)-1,3-phenylene bis(2,2-dimethylpropanoate)

A 20 mL screw cap vial was charged with benzyl 2,4-dihydroxybenzoate (733 mg, 3.00 mmol, prepared as described in Synth. Commun. 2005, 35, 145) and pyridine (3 mL), then cooled in a 0° C. ice bath. Trimethylacetyl chloride (0.739 mL, 6.00 mmol) was added and the reaction mixture was stirred for 16 h. TLC analysis (10% EtOAc in hexanes) showed incomplete conversion to product, so more trimethylacetyl chloride (0.148 mL, 1.20 mmol) was added and after stirring an additional 90 min, the reaction mixture was poured carefully into 1 N HCl (50 mL) at 0° C. The aqueous mixture was extracted with Et₂O (3×50 mL), the combined organic phases were washed with brine, dried over Na₂SO₄ and evaporated. The residue was purified by silica gel chromatography, eluting with EtOAc in hexanes to afford the title compound (1.17 g, 94%) as a clear, colorless oil. ¹H NMR (400 MHz, DMSO-d₆) δ: 7.99 (m, 1H), 7.38 (m, 5H), 7.18 (m, 1H), 7.12 (m, 1H), 5.27 (s, 2H), 1.28 (s, 9H), 1.22 (s, 9H).

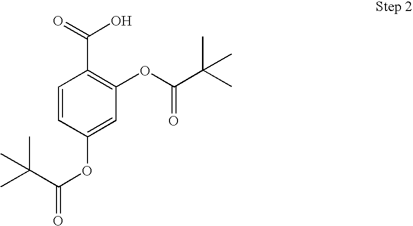

### 2,4-Bis(pivaloyloxy)benzoic acid

A 100 mL round bottom flask was charged with 4-(benzyloxycarbonyl)-1,3-phenylene bis(2,2-dimethylpropanoate) (1.12 g, 2.72 mmol) and EtOAc (14 mL). Nitrogen was bubbled through the reaction mixture, then Pd/C (10%, Degussa type, 250 mg) was added. The flask was flushed with hydrogen and stirred for 16 h. TLC analysis (50% EtOAc in hexanes) showed consumption of starting material. Nitrogen was bubbled through the reaction mixture, then it was then filtered. The filtrate was evaporated and the crude product was purified by silica gel chromatography, eluting with EtOAc in hexanes to afford the title compound (0.765 g, 87%) as a white solid. ¹H NMR (400 MHz, DMSO-d₆) δ: 13.13 (s, 1H), 7.94 (m, 1H), 7.13 (m, 1H), 7.06 (m, 1H), 1.29 (s, 9H), 1.28 (s, 9H).

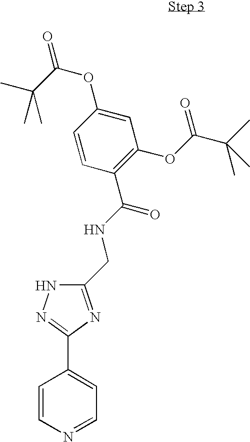

### 4-((3-(Pyridin-4-yl)-1H-1,2,4-triazol-5-yl)methylcarbamoyl)-1,3-phenylene bis(2,2-dimethylpropanoate)

A 50 mL round bottom flask was charged with (3-(pyridin-4-yl)-1H-1,2,4-triazol-5-yl)methanamine dihydrochloride (496 mg, 2.00 mmol, prepared as described in Example 29, Step 1), 2,4-bis(pivaloyloxy)benzoic acid (645 mg, 2.00 mmol), N,N′-dicyclohexycarbodiimide (413 mg, 2.00 mmol), 1-hydroxybenzotriazole hydrate (15 mg, 0.098 mmol), triethylamine (0.558 mL, 4.00 mmol), and a 3:1 mixture of THF and DMF (12 mL). The resulting mixture was stirred overnight at room temperature, filtered, and concentrated under reduced pressure. The crude product was purified by silica gel chromatography, eluting with 10% methanol and CH₂Cl₂ to afford the title compound (0.140 g, 15%) as a dry film. ¹H NMR (400 MHz, CDCl₃) δ: 8.62 (m, 2H), 8.07 (m, 2H), 7.79 (m, 1H), 7.64 (bm, 1H), 7.05 (m, 1H), 6.89 (m, 1H), 4.77 (d, 2H), 1.34 (s, 9H), 1.32 (s, 9H). LCMS (M+1⁺): 479.78.

### EXAMPLE 38

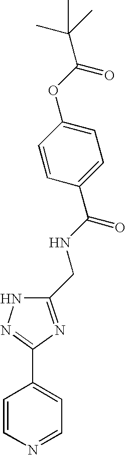

### 4-((3-(Pyridin-4-yl)-1H-1,2,4-triazol-5-yl)methylcarbamoyl)phenyl pivalate

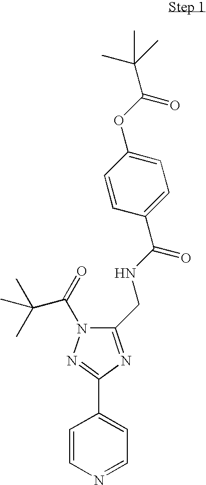

### 4-((1-Pivaloyl-3-(pyridin-4-yl)-1H-1,2,4-triazol-5-yl)methylcarbamoyl)phenyl pivalate

An 8 mL screw cap vial was charged with 4-hydroxy-N-((3-(pyridin-4-yl)-1H-1,2,4-triazol-5-yl)methyl)benzamide (158 mg, 0.535 mmol, prepared as described in Example 28), pyridine (1.1 mL), and trimethylacetyl chloride (0.198 mL, 1.61 mmol), then stirred for 16 h. LCMS analysis showed incomplete consumption of starting material, so more trimethylacetyl chloride (0.132 mL, 1.07 mmol) and pyridine (0.54 mL) were added. After stirring an additional 3 h, the reaction mixture was evaporated and taken up in EtOAc (90 mL), washed with water (2×30 mL), brine (30 mL), dried over Na₂SO₄, and evaporated. The crude product was purified by silica gel chromatography, eluting with EtOAc in hexanes to afford the title compound (170 mg, 69%) as a faintly brown oil.

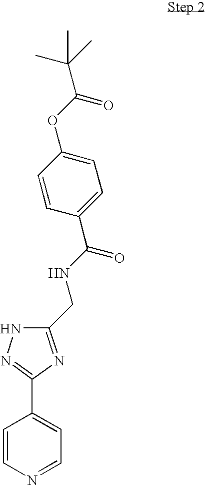

### 4-((3-(Pyridin-4-yl)-1H-1,2,4-triazol-5-yl)methylcarbamoyl)phenyl pivalate

A 20 mL screw cap vial was charged with 4-((1-pivaloyl-3-(pyridin-4-yl)-1H-1,2,4-triazol-5-yl)methylcarbamoyl)phenyl pivalate (158 mg, 0.341 mmol), methanol (3.5 mL) and NaHCO₃ (86 mg, 1.0 mmol), then stirred for 2h. LCMS analysis showed clean conversion. The reaction mixture was filtered and the filter cake was washed with methanol (2×2 mL). The combined filtrates were evaporated and the crude product was purified by silica gel chromatography, eluting with 10% methanol and CH₂Cl₂ to afford the title compound (98 mg, 76%) as clear, colorless, dry film. ¹H NMR (400 MHz, DMSO-d₆) δ: 8.51 (m, 2H), 7.82 (m, 2H), 7.74 (m, 2H), 7.07 (m, 2H), 4.48 (bd, 2H), 1.17 (s, 9H). LCMS (M+1⁺): 380.38.

### EXAMPLE 39

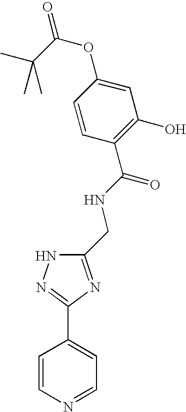

### 3-Hydroxy-4-((3-(pyridin-4-yl)-1H-1,2,4-triazol-5-yl)methylcarbamoyl)phenyl pivalate

A 4 mL screw cap vial was charged with 4-((3-(pyridin-4-yl)-1H-1,2,4-triazol-5-yl)methylcarbamoyl)-1,3-phenylene bis(2,2-dimethylpropanoate) (98 mg, 0.20 mmol, prepared as described in Example 37), a solution of water (5%) and methanol (1.6 mL), and NaHCO₃ (85.8 mg, 1.02 mmol). After stirring for 60 h, the reaction mixture was diluted with methanol (6 mL) and filtered. The filter cake was washed with methanol (8 mL, 4 mL), and dried onto SiO₂ for purification by silica gel chromatography, eluting with 10% methanol in CH₂Cl₂ to afford the title compound (56 mg, 69%) as a white solid. ¹H NMR (400 MHz, DMSO-d₆) δ: 12.52 (b, 1H), 9.48 (bm, 1H), 8.68 (m, 2H), 7.98 (m, 1H), 7.91 (m, 2H), 6.71 (m, 2H), 4.69 (bd, 2H), 1.30 (s, 9H). LCMS (M+1⁺): 396.04.

### EXAMPLE 40

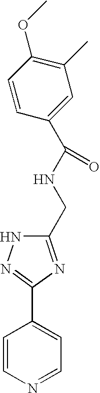

### 4-Methoxy-3-methyl-N-((3-(pyridin-4-yl)-1H-1,2,4-triazol-5-yl)methyl)benzamide

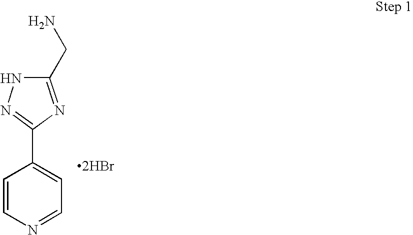

### (3-(Pyridin-4-yl)-1H-1,2,4-triazol-5-yl)methanamine, hydrobromide

A 100 mL round bottom flask was charged with tert-butyl (3-(pyridin-4-yl)-1H-1,2,4-triazol-5-yl)methylcarbamate (165 mg, 0.599 mmol, prepared as described in Example 16), HBr in acetic acid (33%, 4 mL), then stirred in an oil bath at 65° C. for 6 h. The reaction mixture was evaporated with a stream of nitrogen, then from methanol several times, and then dried under vacuum, giving the product as a tan solid (186 mg 71% based on FW for dihydrobromide). ¹H NMR (400 MHz, DMSO-d₆) δ: 9.09 (m, 2H), 8.64 (bs, 3H), 8.49 (m, 2H), 4.33 (bd, 2H).

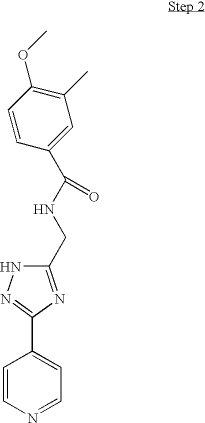

### 4-Methoxy-3-methyl-N-((3-(pyridin-4-yl)-1H-1,2,4-triazol-5-yl)methyl)benzamide

A 25 mL round bottom flask was charged with (3-(pyridin-4-yl)-1H-1,2,4-triazol-5-yl)methanamine hydrobromide (80 mg, 0.24 mmol), 4-methoxy-3-methylbenzoic acid (37 mg, 0.22 mmol), N,N′-dicyclohexycarbodiimide (45 mg, 0.22 mmol), 1-hydroxybenzotriazole hydrate (2 mg, 0.01 mmol), 4-methylmorpholine (0.121 mL, 1.10 mmol), and acetonitrile (1.5 mL). The resulting mixture was stirred for 2 h, then became a thick slurry, so more acetonitrile (1.5 mL) was added. The resulting mixture was stirred overnight, filtered to remove dicyclohexyl urea, concentrated, and purified by C18 reverse phase semi-preparative HPLC, giving the product as a colorless film (mono TFA salt, 6 mg, 8%). ¹H NMR (400 MHz, CD₃OD) δ: 8.82 (m, 2H), 8.48 (m, 2H), 7.78 (m, 1H), 7.72 (m, 1H), 6.99 (m, 1H), 4.77 (s, 2H), 3.89 (s, 3H), 2.23 (s, 3H). LCMS (M+1⁺): 323.90.

### EXAMPLE 41

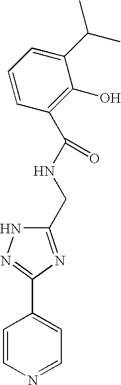

### 2-Hydroxy-3-isopropyl-N-((3-(pyridin-4-yl)-1H-1,2,4-triazol-5-yl)methyl)benzamide

The title compound was prepared analogously to 4-methoxy-3-methyl-N-((3-(pyridin-4-yl)-1H-1,2,4-triazol-5-yl)methyl)benzamide in Example 40, where 2-hydroxy-3-isopropylbenzoic acid, was substituted for 4-methoxy-3-methylbenzoic acid in step 2 of that sequence. ¹H NMR (400 MHz, CD₃CN) 6: 12.68 (bs, 1H), 8.75 (m, 2H), 8.51 (m, 2H), 8.12 (bm, 1H), 7.50 (m, 1H), 7.42 (m, 1H), 6.90 (m, 1H), 4.79 (d, 2H), 3.32 (m, 1H), 1.22 (d, 6H). LCMS (M+1⁺): 337.79

### EXAMPLE 42

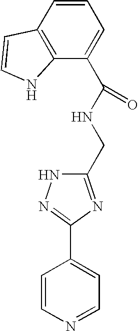

### N-((3-(Pyridin-4-yl)-1H-1,2,4-triazol-5-yl)methyl)-1H-indole-7-carboxamide

The title compound was prepared analogously to 4-methoxy-3-methyl-N-((3-(pyridin-4-yl)-1H-1,2,4-triazol-5-yl)methyl)benzamide in Example 40, where 1H-indole-7-carboxylic acid, was substituted for 4-methoxy-3-methylbenzoic acid in step 2 of that sequence. ¹H NMR (400 MHz, CD₃OD) δ: 8.85 (m, 2H), 8.56 (m, 2H), 7.78 (m, 1H), 7.68 (m, 1H), 7.34 (m, 1H), 7.10 (m, 1H), 6.52 (m, 1H), 4.85 (s, 2H). LCMS (M+1⁺): 319.03

### EXAMPLE 43

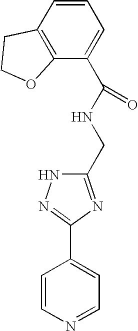

### N-((3-(pyridin-4-yl)-1H-1,2,4-triazol-5-yl)methyl)-2,3-dihydrobenzofuran-7-carboxamide

The title compound was prepared analogously to 4-methoxy-3-methyl-N-((3-(pyridin-4-yl)-1H-1,2,4-triazol-5-yl)methyl)benzamide in Example 40, where 2,3-dihydrobenzofuran-7-carboxylic acid, was substituted for 4-methoxy-3-methylbenzoic acid in step 2 of that sequence. ¹H NMR (400 MHz, CD₃OD) δ: 8.85 (m, 2H), 8.54 (m, 2H), 7.76 (m, 1H), 7.43 (m, 1H), 6.97 (m, 1H), 4.85 (s, 2H), 4.79 (t, 2H), 3.30 (m, 2H). LCMS (M+1⁺): 322.30

### EXAMPLE 44

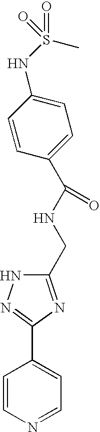

### 4-(Methylsulfonamido)-N-((3-(pyridin-4-yl)-1H-1,2,4-triazol-5-yl)methyl)benzamide

The title compound was prepared analogously to 4-methoxy-3-methyl-N-((3-(pyridin-4-yl)-1H-1,2,4-triazol-5-yl)methyl)benzamide in Example 40, where 4-(methylsulfonamido)benzoic acid (prepared as described in Bioorg. Med. Chem. Lett. 1997, 5, 1873) was substituted for 4-methoxy-3-methylbenzoic acid and DMF was substituted for acetonitrile in step 2 of that sequence. ¹H NMR (400 MHz, DMSO-d₆) δ: 10.15 (s, 1H), 9.13 (bm, 1H), 8.81 (m, 2H), 8.20 (m, 2H), 7.88 (m, 2H), 7.37 (m, 2H), 4.64 (m, 2H), 3.06 (s, 3H). LCMS (M+1¹): 373.03

### EXAMPLE 45

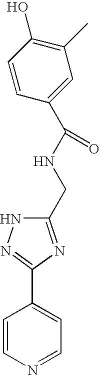

### 4-Hydroxy-3-methyl-N-((3-(pyridin-4-yl)-1H-1,2,4-triazol-5-yl)methyl)benzamide

An 8 mL screw cap vial was charged with (3-(pyridin-4-yl)-1H-1,2,4-triazol-5-yl)methanamine, hydrobromide (67 mg, 0.20 mmol, prepared as described in Example 40), 4-hydroxy-3-methylbenzoic acid (30 mg, 0.20 mmol), N,N′-dicyclohexycarbodiimide (41 mg, 0.20 mmol), 1-hydroxybenzotriazole hydrate (2 mg, 0.01 mmol), 4-methylmorpholine (0.044 mL, 0.40 mmol), and a 3:1 mixture of THF and DMF (1.2 mL). The resulting mixture was stirred for 16 h, at which time it was a thick slurry. The reaction was filtered. The filter cake was washed with THF (3 mL) and methanol (2×2 mL). The filtrate was concentrated and purified by C18 reverse phase semi-preparative HPLC, giving the product as a white solid (mono TFA salt, 25 mg, 30%). ¹H NMR (400 MHz, DMSO-d₆) δ: 9.93 (bs, 1H), 8.89 (bm, 1H), 8.79 (m, 2H), 8.17 (m, 2H), 7.68 (m, 1H), 7.60 (m, 1H), 6.81 (m, 1H), 4.60 (bd, 2H), 2.14 (s, 3H). LCMS (M+1⁺): 310.09.

### EXAMPLE 46

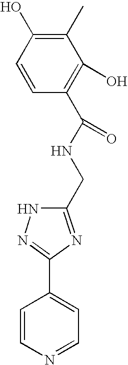

### 2,4-Dihydroxy-3-methyl-N-((3-(pyridin-4-yl)-1H-1,2,4-triazol-5-yl)methyl)benzamide

An 8 mL screw cap vial was charged with (3-(pyridin-4-yl)-1H-1,2,4-triazol-5-yl)methanamine, hydrobromide (67 mg, 0.20 mmol, prepared as described in Example 40), 2,4-dihydroxy-3-methylbenzoic acid (34 mg, 0.20 mmol, prepared as described in J. Chem. Soc. Perkin Trans. 1 1988, 839), N,N′-dicyclohexycarbodiimide (41 mg, 0.20 mmol), 1-hydroxybenzotriazole hydrate (2 mg, 0.01 mmol), triethylamine (0.056 mL, 0.40 mmol), and a 3:1 mixture of THF and DMF (1.2 mL). The resulting mixture was stirred for 40 h, and filtered to remove dicylohexyl urea. The filtrate was concentrated and purified by silica gel chromatography, eluting with 10% methanol in CH₂Cl₂, then further purified by C18 reverse phase semi-preparative HPLC, giving the product as a film (mono TFA salt, 15 mg, 17%). ¹H NMR (400 MHz, CD₃OD) δ: 8.87 (m, 2H), 8.60 (m, 2H), 7.46 (d, 1H), 6.38 (d, 1H), 4.76 (s, 2H), 2.03 (s, 3H). LCMS (M+1⁻): 326.03.

### EXAMPLE 47

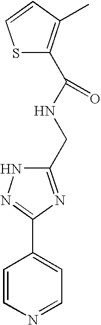

### 3-Methyl-N-((3-(pyridin-4-yl)-1H-1,2,4-triazol-5-yl)methyl)thiophene-2-carboxamide

A 5 mL round bottom flask was charged with (3-(pyridin-4-yl)-1H-1,2,4-triazol-5-yl)methanamine (81.5 mg, 0.242 mmol, prepared as described in Example 16), 3-methyl-2-thiopene carboxylic acid (34.4 mg, 0.242 mmol), DCC (49.9 mg, 0.242 mmol), HOBt (1.9 mg, 0.0121 mmol), triethylamine (49.0 mg, 0.132 mmol), and THF:DMF (1 mL, 4:1). The resulting mixture was stirred overnight at room temperature. Work-up: the mixture was diluted with EtOAc (50 mL), washed three times with water (50 mL), brine (50 mL), and dried over Na₂SO₄. The crude material was purified by C18 reverse phase semi-preparative HPLC, giving the product as white solid (bis TFA salt, 68.6 mg, 95%). ¹H NMR (400 MHz, DMSO-d₆) δ: 8.84 (d, 2H), 8.58 (t, 1H), 8.23 (d, 2H), 7.59 (d, 1H), 6.96 (d, 1H), 4.59 (d, 2H), 2.44 (s, 3H). LCMS (M+1)⁺: 300.46.

### EXAMPLE 48

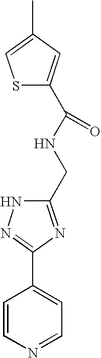

### 4-Methyl-N-((3-(pyridin-4-yl)-1H-1,2,4-triazol-5-yl)methyl)thiophene-2-carboxamide

The title compound was prepared analogously to 3-methyl-N-((3-(pyridin-4-yl)-1H-1,2,4-triazol-5-yl)methyl)thiophene-2-carboxamide, where 4-methyl-2-thiopene carboxylic acid was substituted for 3-methyl-2-thiopene carboxylic acid as described in Example 47. ¹H NMR (400 MHz, DMSO-d₆) δ: 9.14 (t, 1H), 8.79 (d, 2H), 8.17 (d, 2H), 7.63 (d, 1H), 7.37 (d, 1H), 4.60 (d, 2H), 2.21 (s, 3H). LCMS (M+1)⁺: 300.38.

### EXAMPLE 49

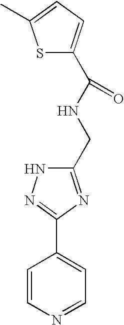

### 5-Methyl-N-((3-(pyridin-4-yl)-1H-1,2,4-triazol-5-yl)methyl)thiophene-2-carboxamide

The title compound was prepared analogously to 3-methyl-N-((3-(pyridin-4-yl)-1H-1,2,4-triazol-5-yl)methyl)thiophene-2-carboxamide, where 5-methyl-2-thiopene carboxylic acid was substituted for 3-methyl-2-thiopene carboxylic acid as described in Example 47. ¹H NMR (400 MHz, DMSO-d₆) δ: 9.08 (t, 1H), 8.78 (d, 2H), 8.13 (d, 2H), 7.61 (d, 1H), 6.85 (d, 1H), 4.59 (d, 2H), 2.45 (s, 3H). LCMS (M+1)⁺: 300.64.

### EXAMPLE 50

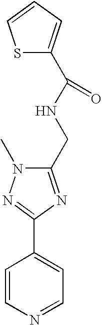

### N-((1-Methyl-3-(pyridin-4-yl)-1H-1,2,4-triazol-5-yl)methyl)thiophene-2-carboxamide

A 5 mL round bottom flask was charged with N-((3-(pyridin-4-yl)-1H-1,2,4-triazol-5-yl)methyl)thiophene-2-carboxamide (20.1 mg, 0.0503 mmol, prepared as described in Example 16), (trimethylsilyl)diazomethane (2.0 M solution in Et₂O, 5.74 mg, 0.0503 mmol), and THF:MeOH (0.5 mL, 1:1). The resulting mixture was stirred 1 hour at room temperature. The mixture was concentrated, and then purified by SiO₂ flash chromatography, eluting with 10% methanol and methylene chloride to afford the title compound in 6.1 mg (40% yield),as a yellow solid. ¹H NMR (400 MHz, DMSO-d₆) δ: 8.59 (d, 2H), 8.01 (d, 2H), 7.74 (d, 1H), 7.68 (d, 1H), 7.13 (t, 1H), 4.76 (d, 2H), 4.05 (s, 3H). LCMS (M+1)⁺: 300.03.

### EXAMPLE 51

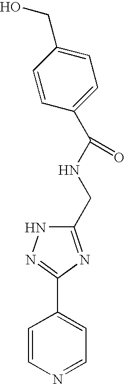

### 4-(Hydroxymethyl)-N-((3-(pyridin-4-yl)-1H-1,2,4-triazol-5-yl)methyl)benzamide

The title compound was prepared analogously to 3-methyl-N-((3-(pyridin-4-yl)-1H-1,2,4-triazol-5-yl)methyl)thiophene-2-carboxamide, where 4-(hydroxymethyl)benzoic acid was substituted for 3-methyl-2-thiopene carboxylic acid as describe in Example 47 ¹H NMR (400 MHz, DMSO-d₆) δ: 9.14 (t, 1H), 8.63 (d, 2H), 7.89 (d, 2H), 7.87 (d, 2H), 7.40 (d, 2H), 5.30 (bs, 1H), 4.62 (d, 2H), 4.54 (s, 2H). LCMS (M+1)⁺: 309.99.

### EXAMPLE 52

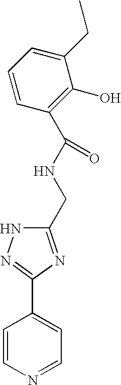

### 3-Ethyl-2-hydroxy-N-((3-(pyridin-4-yl)-1H-1,2,4-triazol-5-yl)methyl)benzamide

The title compound was prepared analogously to 3-methyl-N-((3-(pyridin-4-yl)-1H-1,2,4-triazol-5-yl)methyl)thiophene-2-carboxamide, where 3-ethyl-2-hydroxybenzoic acid (prepared as described in Tetrahedron, 1987, 43, 1519-1540.) was substituted for 3-methyl-2-thiopene carboxylic acid as describe in Example 47 ¹H NMR (400 MHz, CD₃OD) δ: 8.85 (d, 2H), 8.57 (d, 2H), 7.61 (dd, 1H), 7.31 (dd, 1H), 6.82 (t, 1H), 4.80 (s, 2H), 2.65 (q, 2H), 1.21 (t, 3H). LCMS (M+1)⁺: 323.87.

### EXAMPLE 53

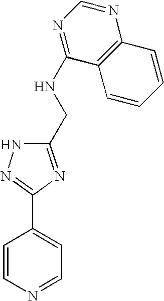

### N-((3-(Pyridin-4-yl)-1H-1,2,4-triazol-5-yl)methyl)quinazolin-4-amine

A 5 mL round bottom flask was charged with (3-(pyridin-4-yl)-1H-1,2,4-triazol-5-yl)methanamine (94 mg, 0.382 mmol, prepared as described in Example 16), 4-chloroquinazoline (52 mg, 0.318 mmol), triethylamine (96 mg, 0.954 mmol), and DMF. The resulting mixture was stirred for 2 hours at 80° C. The crude material was purified by C18 reverse phase semi-preparative HPLC. ¹H NMR (400 MHz, CD₃OD) δ: 8.87 (d, 2H), 8.84 (s, 1H), 8.57 (d, 2H), 8.44 (dd, 1H), 8.11 (dt, 1H), 7.87 (m, 2H), 5.29 (s, 2H). LCMS (M+1)⁺: 303.94.

### EXAMPLE 54

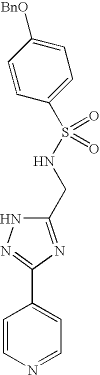

### 4-(Benzyloxy)-N-((3-(pyridin-4-yl)-1H-1,2,4-triazol-5-yl)methyl)benzenesulfonamide

A 5 mL round bottom flask was charged with (3-(pyridin-4-yl)-1H-1,2,4-triazol-5-yl)methanamine (111 mg, 0.448 mmol, prepared as described in Example 16), 4-benzyloxy benzene sulfonyl chloride (126 mg, 0.448 mmol), triethylamine (135 mg, 0.1.33 mmol), and DMF. The resulting mixture was stirred for 1 hour at room temperature. Work-up: the mixture was diluted with EtOAc (50 mL), washed three times with water (50 mL), brine (50 mL), and dried over Na₂SO₄. The mixture was concentrated, and purified by SiO₂ flash chromatography, eluting with 10% methanol and methylene chloride to afford the title compound in 136 mg (72% yield), as a white solid. ¹H NMR (400 MHz, DMSO-d₆) δ: 8.64 (d, 2H), 8.22 (bs, 1H), 7.81 (dd, 2H), 7.69 (d, 2H), 7.69 (d, 2H), (bs, 1H), 7.39-7.31 (m, 5H), 7.11 (d, 2H), 5.07 (s, 2H), 4.10 (s, 2H). LCMS (M+1)⁻: 422.13.

### EXAMPLE 55

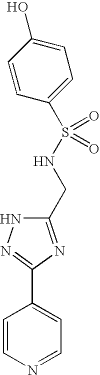

### 4-Hydroxy-N-((3-(pyridin-4-yl)-1H-1,2,4-triazol-5-yl)methyl)benzenesulfonamide

A 5 mL round bottom flask was charged with 4-(benzyloxy)-N-((3-(pyridin-4-yl)-1H-1,2,4-triazol-5-yl)methyl)benzenesulfonamide (126 mg, 0.299 mmol, prepared as described in Example 54), 10% wet palladium on carbon (3 mg, 0.0299 mmol) in EtOAc. The resulting mixture was degassed thoroughly with gaseous N₂, then saturated with gaseous H₂ and stirred for 30 hours at room temperature. Work-up: the mixture was filtered through a pad of celite, concentrated, and then purified by SiO₂ flash chromatography, eluting with 10% methanol and methylene chloride to afford the title compound in 25 mg (25% yield),as a white solid ¹H NMR (400 MHz, DMSO-d₆) δ: 10.35 (bs, 1H), 8.65 (d, 2H), 8.07 (bs, 1H), 7.87 (d, 2H), 7.61 (d, 2H), 6.85 (d, 2H), 4.07 (s, 2H), LCMS (M+1)⁺: 332.00.

### EXAMPLE 56

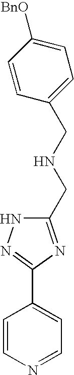

### N-(4-(Benzyloxy)benzyl)-1-(3-(pyridin-4-yl)-1H-1,2,4-triazol-5-yl)methanamine

A 5 mL round bottom flask was charged with (3-(pyridin-4-yl)-1H-1,2,4-triazol-5-yl)methanamine (122 mg, 0.492 mmol, prepared as described in Example 16), 4-benzyloxy benzyl bromide (136 mg, 0.492 mmol), triethylamine (149 mg, 0.1.475 mmol), and DMF. The resulting mixture was stirred for 10 min. at room temperature. Work-up: the mixture was diluted with EtOAc (50 mL), washed three times with water (50 mL), brine (50 mL), and dried over Na₂SO₄. The mixture was concentrated, and then purified by SiO₂ flash chromatography, eluting with 10% methanol and methylene chloride to afford the title compound in 37 mg (20% yield), as a off white solid. ¹H NMR (400 MHz, CD₃OD) δ: 8.62 (dd, 2H), 8.01 (dd, 2H), 7.42-7.27 (m, 7H), 6.95 (m, 2H), 5.05 (s, 2H), 3.97 (s, 2H). LCMS (M+1)⁺: 372.06.

### EXAMPLE 57

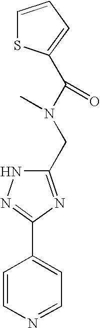

### N-Methyl-N-((3-(pyridin-4-yl)-1H-1,2,4-triazol-5-yl)methyl)thiophene-2-carboxamide

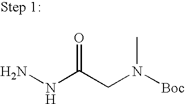

### tert-Butyl 2-hydrazinyl-2-oxoethyl(methyl)carbamate

A 250 mL round bottom flask was charged with 2-(tert-butoxycarbonyl(methyl)amino)acetic acid (5.17 g, 27.3 mmol), N-methyl morpholine (3.59 g, 35.5 mmol), and THF (110 mL), was cooled to 0° C. To the mixture was added dropwise ethyl chloroformate (3.26 g, 30.1 mmol) while stirring the mixture for 30 min. at 0° C. The reaction mixture was then filtered to remove N-methyl morpholinium salts. The resulting solution was added drop wise to a solution of hydrazine (4.38 g, 136 mmol) in THF cooled to 0° C. The resulting mixture was stirred 1 hour allowing the reaction mixture to warm to room temperature. Work-up: the mixture was diluted with EtOAc (50 mL), washed three times with water (50 mL), brine (50 mL), and dried over Na₂SO₄. The white solid crude product (1.30 g, 22%) was used in the next step without further purification. ¹H NMR (400 MHz, DMSO-d₆) δ: 9.01 (m, 1H), 4.19 (bs, 2H), 3.69 (d, 2H), 2.76 (m, 3H), 1.34 (m, 9H).

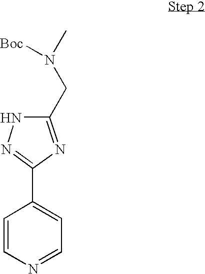

### tert-Butyl methyl((3-(pyridin-4-yl)-1H-1,2,4-triazol-5-yl)methyl)carbamate

A 20 mL microwave reaction vessel was charged with tert-butyl 2-hydrazinyl-2-oxoethyl(methyl)carbamate (528 mg, 2.41 mmol), 4-cyanopyridine (753 mg, 7.23 mmol), K₂CO₃ (166 mg, 1.20 mmol), and n-butanol. The resulting mixture was heated in the microwave for 10 min. at 170° C. The reaction mixture was then filtered to remove insoluble salts, the filtrate was then concentrated and purified by SiO₂ flash chromatography, eluting with 10% methanol and methylene chloride to afford the title compound in 656 mg (94% yield),as a white solid. (M+1)⁺: 289.85.

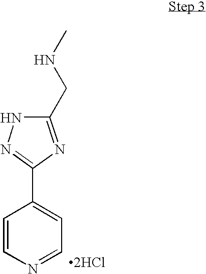

### N-Methyl-1-(3-(pyridin-4-yl)-1H-1,2,4-triazol-5-yl)methanamine hydrochloride

A 15 mL round bottom flask was charged with tert-butyl methyl((3-(pyridin-4-yl)-1H-1,2,4-triazol-5-yl)methyl)carbamate (875 mg, 3.02 mmol), and 4 M HCl in dioxane (7 mL). The resulting mixture was stirred at reflux for 4 hours. Work-up: the mixture was concentrated, and used in the next step without further purification. ¹H NMR (400 MHz, DMSO-d₆) δ: 9.64 (bs, 1H), 8.93 (dd, 2H), 8.34 (d, 2H), 4.39 (s, 2H), 2.96 (s, 1H), 2.68 (s, 3H). (M+1)⁺: 190.08.

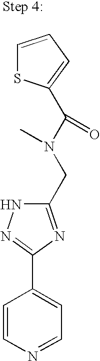

### N-Methyl-N-((3-(pyridin-4-yl)-1H-1,2,4-triazol-5-yl)methyl)thiophene-2-carboxamide

A 5 mL round bottom flask was charged with N-methyl-1-(3-(pyridin-4-yl)-1H-1,2,4-triazol-5-yl)methanamine hydrochloride (119 mg, 0.454 mmol), thiopene-2-carboxylic acid (58 mg, 0.454 mmol), N,N′-dicyclohexylcarbodiimide (94 mg, 0.454 mmol), N-hydroxybenzotriazole (4 mg, 0.0227 mmol), triethylamine (138 mg, 1.36 mmol), and THF:DMF (2 mL, 2:1). The resulting mixture was stirred overnight at room temperature. Work-up: the mixture was diluted with EtOAc (50 mL), washed three times with water (50 mL), brine (50 mL), and dried over Na₂SO₄. The crude material was purified by C18 reverse phase semi-preparative HPLC, giving the product as white solid (bis TFA salt, 90 mg, 67%). ¹H NMR (400 MHz, CD₃OD) δ: 8.84 (d, 2H), 8.51 (d, 2H), 7.69 (d, 1H), 7.61 (d, 1H), 7.14 (t, 1H), 4.97 (s, 2H), 3.48 (bs, 3H). LCMS (M+1⁺): 300.58.

### EXAMPLE 58

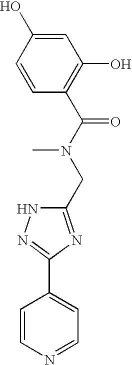

### 2,4-Dihydroxy-N-methyl-N-((3-(pyridin-4-yl)-1H-1,2,4-triazol-5-yl)methyl)benzamide

The title compound was prepared analogously to N-methyl-N-((3-(pyridin-4-yl)-1H-1,2,4-triazol-5-yl)methyl)thiophene-2-carboxamide, where 2,4-dihydroxybenzoic acid was substituted for thiopene-2-carboxylic acid as describe in Example 57. ¹H NMR (400 MHz, DMSO-d₆) δ: 8.88 (d, 2H), 8.31 (d, 2H), 7.01 (d, 1H), 6.29 (d, 1H), 6.21 (d, 1H), 4.74 (s, 2H), 2.99 (s, 3H). LCMS (M+1⁺): 325.95.

### EXAMPLE 59

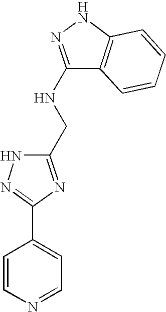

### N-((3-(Pyridin-4-yl)-1H-1,2,4-triazol-5-yl)methyl)-1H-indazol-3-amine

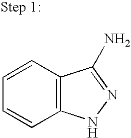

### 1H-Indazol-3-amine

A 1L round bottom flask charged with 2-fluorobenzonitrile (40 g, 0.331 mol), hydrazine monohydrate (50 g, 1.0 mol), and n-butanol (300 mL). The resulting solution was heated to reflux and stirred overnight. Reaction progress was monitored by TLC (EtOAc/Et₃N=1000/1). Work up: the mixture was concentrated to a slurry, and filtered to afford the title compound in 24.0 g (55% yield) as a white solid. ¹H NMR (300 MHz, DMSO-d₆) δ: 11.35 (s, 1H), 7.67 (d, 1H), 7.23-7.19 (m, 2H), 6.95-6.85 (m, 1H), 5.32 (s, 2H). LCMS (M+1⁺): 134.

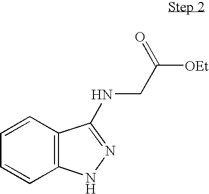

### Ethyl 2-(1H-indazol-3-ylamino)acetate

A 1L round bottom flask was charged with 1H-indazol-3-amine (24 g, 180mmol), K₂CO₃ (50 g, 362 mmol), and DMF (300 mL). To the reaction mixture, ethyl 2-bromoacetate (30.1 g, 180 mmol) in DMF (50 mL), was added drop wise at 80° C. The resulting mixture was stirred overnight at 80° C. Reaction progress was monitored by TLC(EtOAc/PE=1/5). Work-up: the mixture was diluted with EtOAc (300 mL), washed three times with water (100 mL), brine (100 mL), and dried over Na₂SO₄. The crude material was purified by SiO₂ chromatography eluting with EtOAc and petroleum ether, giving the product as white solid. The final product was additionally recrystallized from EtOAc/petroleum ether (1/4), giving the product as a white solid (3.4 g, 9%). ¹H NMR (300 MHz, DMSO-d₆) δ: 11.44 (s, 1H), 7.73 (d, 1H), 7.27-7.21 (m, 2H), 6.96-6.86 (m, 1H), 6.53 (t, 1H), 4.09 (q, 2H), 4.01 (d, 2H), 1.19 (t, 3H). LCMS (M+1⁺): 220.

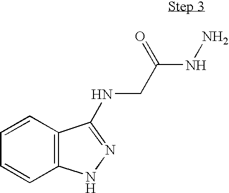

### 2-(1H-Indazol-3-ylamino)acetohydrazide

To a 15 mL round bottom flask charged with ethyl 2-(1H-indazol-3-ylamino)acetate (484 mg, 2.21 mmol) and ethanol (4 mL), was added hydrazine (354 mg, 11.0 mmol) dropwise. The resulting mixture was stirred overnight at room temperature. Work-up: the mixture was concentrated giving the product as white solid (408 mg, 90%) and used in the next step without further purification. ¹H NMR (400 MHz, DMSO-d₆) δ: 11.42 (s, 1H), 8.99 (bs, 1H), 7.71 (d, 1H), 7.21 (d, 2H), 6.91-6.87 (m, 1H), 6.23 (t, 1H), 4.17 (bs, 2H), 3.80 (d, 2H). LCMS (M+1⁺): 206.09.

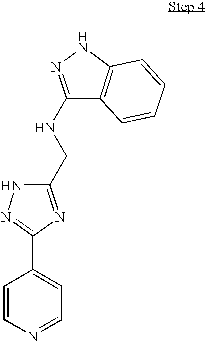

### N-((3-(Pyridin-4-yl)-1H-1,2,4-triazol-5-yl)methyl)-1H-indazol-3-amine

A 20 mL sealed tube was charged with 2-(1H-indazol-3-ylamino)acetohydrazide (232 mg, 1.06 mmol), 4-cyanopyridine (331 mg, 3.17 mmol), DBU (161 mg, 1.06 mmol) in n-butanol (4 mL). The resulting mixture was heated for 4 hours at 140° C. The crude material was purified by C18 reverse phase semi-preparative HPLC, giving the product as light a brown solid (bis TFA salt, 27 mg, 9%). ¹H NMR (400 MHz, DMSO-d₆) δ: 11.52 (s, 1H), 8.81 (d, 2H), 8.20 (d, 2H), 7.74 (d, 1H), 7.24 (d, 2H), 7.09 (s, 1H), 6.96-6.91 (m, 1H), 4.65 (s, 2H). LCMS: 292.16 (M+1⁺).

### EXAMPLE 60

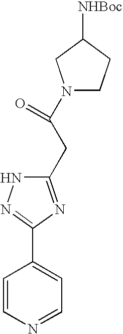

### 1-(3-Aminopyrrolidin-1-yl)-2-(3-(pyridin-4-yl)-1H-1,2,4-triazol-5-yl)ethanone

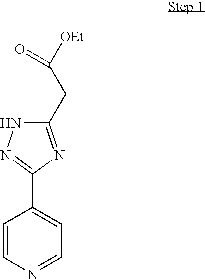

### Ethyl 2-(3-(pyridin-4-yl)-1H-1,2,4-triazol-5-yl)acetate

A 500 mL round bottom flask was charged with isonicotinic acid hydrazide (2.19 g, 16.0 mmol), ethyl 3-ethoxy-3-iminopropanoate hydrochloride (6.27 g, 32.0 mmol), and ethanol (192 mL). The resulting solution was heated to 91° C. for 4 days. Work-up: mixture was concentrated, and purified by SiO₂ flash chromatography, eluting with EtOAc and hexanes to afford the title compound, 2.79 g (75% yield) as an pale yellow solid. LCMS (M+1)⁻: 233.56.

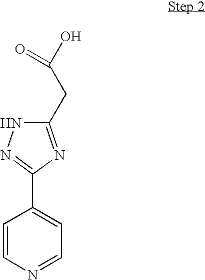

### 2-(3-(Pyridin-4-yl)-1H-1,2,4-triazol-5-yl)acetic acid

A 100 mL round bottom flask, was charged with ethyl 2-(3-(pyridin-4-yl)-1H-1,2,4-triazol-5-yl)acetate (2.78 g, 11.9 mmol), lithium hydroxide (0.316 g, 13.1 mmol), and THF:H₂O (30 mL, 15:1). The resulting solution was allowed to react for 30 min. at room temperature. Work-up: the mixture was filtered, and the collected solid was washed with 25 mL of EtOAc, and dried resulting in 2.41 g (86%) of product as a light yellow solid. LCMS (M+1)⁺: 205.33.

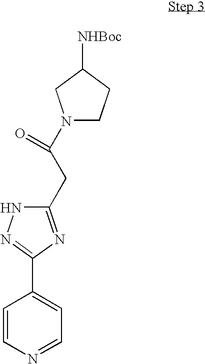

### tert-Butyl 1-(2-(3-(pyridin-4-yl)-1H-1,2,4-triazol-5-yl)acetyl)pyrrolidin-3-ylcarbamate

A 10 mL round bottom flask was charged with 2-(3-(pyridin-4-yl)-1H-1,2,4-triazol-5-yl)acetic acid (128 mg, 0.532 mmol), tert-butyl pyrrolidin-3-ylcarbamate (118 mg, 0.639 mmol), HATU (283 mg, 0.745 mmol), triethylamine (215 mg, 2.13 mmol), DMF (1.1 mL). The resulting mixture was stirred 1 hour at room temperature. Work-up: the mixture was diluted with EtOAc (50 mL), washed with three times with water (50 mL), brine (50 mL), and dried over Na₂SO₄. The mixture was concentrated, and purified by SiO₂ flash chromatography, eluting with (30 to 90%)EtOAc and hexanes to afford the title compound in 98 mg (50% yield) as an pale yellow solid. ¹H NMR (400 MHz, DMSO-d₆) δ: 8.82 (dd, 2H), 7.89 (dd, 2H), 7.27 (bs, 1H), 7.25 (bs, 1H), 4.20 (m, 2H), 4.07 (m, 2H), 3.56 (m, 2H), 3.46 (m, 2H), 3.34 (m, 2H), 1.37 (s, 9H). LCMS (M+1)⁺: 373.96.

### EXAMPLE 61

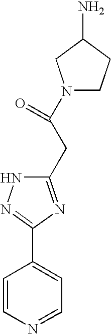

### 1-(3-Aminopyrrolidin-1-yl)-2-(3-(pyridin-4-yl)-1H-1,2,4-triazol-5-yl)ethanone

A 5 mL round bottom flask was charged with tert-butyl 1-(2-(3-(pyridin-4-yl)-1H-1,2,4-triazol-5-yl)acetyl)pyrrolidin-3-ylcarbamate (49 mg, 0.132 mmol, prepared as described in Example 60), in methylene chloride (1 mL), and trifluoroacetic acid (1 mL). The resulting mixture was stirred 5 min. at room temperature. The mixture was concentrated, and purified by C18 reverse phase semi-preparative HPLC, giving the product as pale yellow solid (bis TFA salt, 5.3 mg, 15%). ¹H NMR (400 MHz, DMSO-d₆) δ: 8.86 (dd, 2H), 8.11 (dd, 2H), 7.27 (bs, 1H), 7.25 (bs, 1H), 4.05 (m, 2H), 3.97 (m, 1H), 3.82 (m, 2H), 3.67 (m, 2H), 3.34 (m, 2H). LCMS (M+1)⁺: 273.99

### EXAMPLE 62

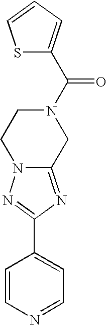

### (2-(Pyridin-4-yl)-5,6-dihydro- [1,2,4]triazolo [1,5-a]pyrazin-7(8H)-yl)(thiophen-2-yl)methanone

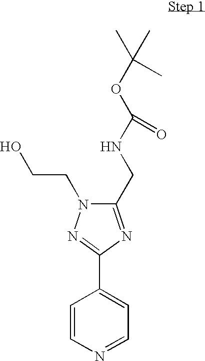

### tert-Butyl (1-(2-hydroxyethyl)-3-(pyridin-4-yl)-1H-1,2,4-triazol-5-yl)methylcarbamate

A 25 mL round bottom flask was charged with tert-butyl (3-(pyridin-4-yl)-1H-1,2,4-triazol-5-yl)methylcarbamate (275 mg, 1.00 mmol, prepared as described in Example 16), 2-bromoethanol (0.213 mL, 3.01 mmol), potassium carbonate (415 mg, 3.00 mmol), and acetonitrile (5 mL), then stirred in an oil bath at 70° C. for 16 h. TLC analysis (10% methanol in EtOAc) showed no triazole starting material. The reaction mixture was filtered, the filter cake was washed with acetonitrile (10 mL) and the combined filtrates were evaporated. The crude residue was then purified by silica gel chromatography, eluting with 10% methanol in CH₂Cl₂ to afford the title compound (197 mg, 62%) as a white solid. ¹H NMR (400 MHz, CD₃OD) δ: 8.61 (m, 2H), 8.03 (m, 2H), 4.50 (s, 2H), 4.42 (bm, 2H), 3.94 (m, 2H), 1.46 (s, 9H). LCMS (M+1⁺): 320.01.

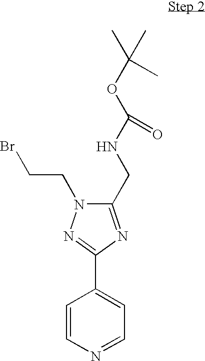

### tert-Butyl (1-(2-bromoethyl)-3-(pyridin-4-yl)-1H-1,2,4-triazol-5-yl)methylcarbamate

An 8 mL screw cap vial was charged with tert-butyl (1-(2-hydroxyethyl)-3-(pyridin-4-yl)-1H-1,2,4-triazol-5-yl)methylcarbamate (230 mg, 0.720 mmol), triphenylphosphine (175 mg, 0.865 mmol), carbon tetrabromide (287 mg, 865 mmol), and acetonitrile (1.8 mL). The resulting mixture was stirred overnight at room temperature. Reaction progress was monitored by LCMS. The reaction mixture was evaporated and the crude product was purified by silica gel chromatography, eluting with 10% methanol in CH₂Cl₂ to afford the title compound (118 mg, 43%) as a white solid. ¹H NMR (400 MHz, CDCl₃) δ: 8.72 (m, 2H), 8.15 (m, 2H), 5.46 (bm, 1H), 4.71 (bm, 2H), 4.55 (d, 2H), 3.80 (t, 2H), 1.44 (s, 9H).

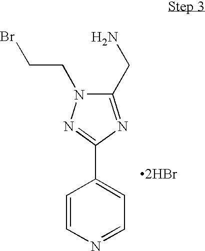

### (1-(2-Bromoethyl)-3-(pyridin-4-yl)-1H-1,2,4-triazol-5-yl)methanamine, hydrobromide

A 4 mL screw cap vial was charged with tert-butyl (1-(2-bromoethyl)-3-(pyridin-4-yl)-1H-1,2,4-triazol-5-yl)methylcarbamate (42 mg, 0.11 mmol), and a solution of HBr in acetic acid (33%, 0.7 mL). The reaction mixture was warmed in a 65° C. oil bath for 16 h, and then cooled. The reaction mixture was blown dry with a stream of nitrogen, and then evaporated several times from methanol providing the title compound (47 mg, 96% based on FW for dihydrobromide) as a tan solid which was used in the next step without further purification.

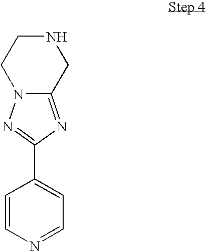

### 2-(Pyridin-4-yl)-5,6,7,8-tetrahydro-[1,2,4]triazolo [1,5-a]pyrazine

A 25 mL round bottom flask was charged with (1-(2-bromoethyl)-3-(pyridin-4-yl)-1H-1,2,4-triazol-5-yl)methanamine hydrobromide (32 mg, 0.072 mmol), 4-methylmorpholine (0.158 mL, 1.44 mmol), and methanol (2.9 mL). The reaction was monitored by LCMS. After 24 hrs. the reaction was evaporated to dryness, providing the title compound, which was used in the next step without further purification. LCMS (M+1⁺): 202.10.

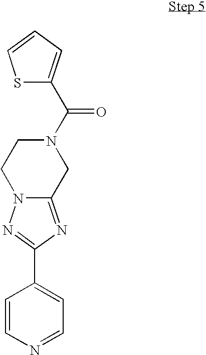

### (2-(Pyridin-4-yl)-5,6-dihydro-[1,2,4]triazolo[1,5-a]pyrazin-7(8H)-yl)(thiophen-2-yl)methanone

A 4 mL screw cap vial was charged with 2-(pyridin-4-yl)-5,6,7,8-tetrahydro-[1,2,4]triazolo[1,5-a]pyrazine (0.072 mmol theoretical yield), thiophene-2-carboxylic acid (9 mg, 0.07 mmol), N,N′-dicyclohexycarbodiimide (14.4 mg, 0.0698 mmol), 1-hydroxybenzotriazole hydrate (1 mg, 0.007 mmol), 4-methylmorpholine (0.0385 mL, 0.350 mmol), and a 1:1 mixture of acetonitrile and DMF (0.4 mL). The resulting mixture was stirred overnight at room temperature, filtered, and evaporated. The crude product was purified by C18 reverse phase semi-preparative HPLC, giving the product as a dry film (mono TFA salt, 20 mg, 65% for two steps). ¹H NMR (400 MHz, CD₃OD) δ: 8.90 (m, 2H), 8.60 (m, 2H), 7.74 (m, 1H), 7.61 (m, 1H), 7.19 (m, 1H), 5.16 (bs, 2H), 4.49 (m, 2H), 4.33 (m, 2H). LCMS (M+1⁺): 311.86.

### EXAMPLE 63

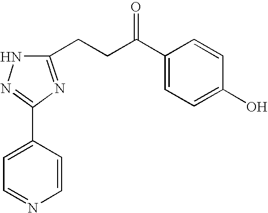

### 1-(4-hydroxyphenyl)-3-(3-(pyridin-4-yl)-1H-1,2,4-triazol-5-yl)propan-1-one

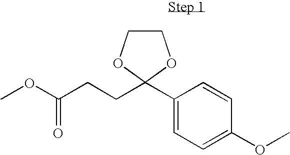

### methyl 3-(2-(4-methoxyphenyl)-1,3-dioxolan-2-yl)propanoate

A 500 mL round bottom flask was charged with methyl 4-(4-methoxyphenyl)-4-oxobutanoate (6.0 g, 27.0 mmol), ethane-1,2-diol (8.4 g, 135.4 mmol), 4-methylbenzenesulfonic acid (2.0 g, 11.6 mmol), and toluene (300 mL). The resulting solution was heated overnight in a 125° C. oil bath. Reaction progress was monitored by TLC (EtOAc/petroleum ether=1:1). Work-up: the mixture was concentrated, dissolved in EtOAc (300 mL), washed with IN NaHCO₃ (2×100 mL), brine, dried over MgSO₄, and concentrated. This resulted in 2 g (26% yield) of product as pale yellow oil that was used without further purification.

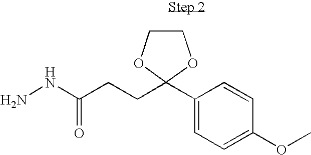

### 3-(2-(4-methoxyphenyl)-1,3-dioxolan-2-yl)propanehydrazide

A 250 mL round bottom flask was charged with methyl 3-(2-(4-methoxyphenyl)-1,3-dioxolan-2-yl)propanoate (2.0 g, 7.52 mmol, prepared as described in Tetrahedron (2003), 59(19), 3487-3491), methanol (100 mL), and hydrazine hydrate (2.35 g, 37.6 mmol, 80% aq). The resulting solution was stirred for 5 h at room temperature. Reaction progress was monitored by TLC (EtOAc/petroleum ether=1/1). Work-up: the mixture was concentrated under vacuum giving the product (2 g, 95% yield), as a white solid that was used without further purification.

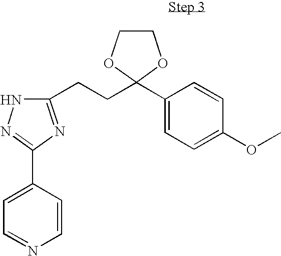

### 4-(5-(2-(2-(4-methoxyphenyl)-1,3-dioxolan-2-yl)ethyl)-1H-1,2,4-triazol-3-yl)pyridine

A 250 mL sealed flask was charged with isonicotinonitrile (2.34 g, 22.51 mmol), 3-(2-(4-methoxyphenyl)-1,3-dioxolan-2-yl)propanehydrazide (2.0 g, 7.51 mmol), K₂CO₃ (520 mg, 3.76 mmol), and n-BuOH (100 mL). The resulting solution was stirred for 12 hours in a 150 deg. C oil bath. Reaction progress was monitored by TLC (CH₂Cl₂/methanol=10/1). Work-up: the mixture was concentrated, dissolved in CH₂Cl₂ (100 mL), filtered, concentrated, and purified by flash chromatography (30/1 CH₂Cl₂/methanol). This gave 2.5 g(94% yield) of product as a light yellow solid. LCMS (M+1)⁺: 353.1.

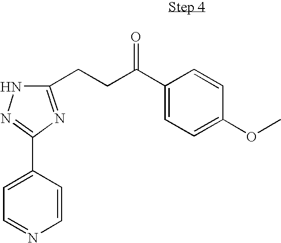

### 1-(4-methoxyphenyl)-3-(3-(pyridin-4-yl)-1H-1,2,4-triazol-5-yl)propan-1-one

A 100 mL round bottom flask was charged with 4-(5-(2-(2-(4-methoxyphenyl)-1,3-dioxolan-2-yl)ethyl)-1H-1,2,4-triazol-3-yl)pyridine (500 mg, 1.42 mmol), EtOH (50 mL), and HCl (5 mL, 30% aq.). The resulting solution was stirred for 2 hours at room temperature. Reaction progress was monitored by TLC (CH₂Cl₂/methanol=10/1). Work-up: the mixture was concentrated, pH adjusted to 12 with NaOH (1N aq.), and filtered. The filter cake was washed water (2×20 mL), and dried in an oven under reduced pressure, giving 0.40 g, 91% yield of product as a white solid.

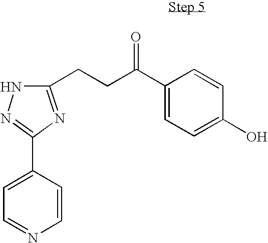

### 1-(4-hydroxyphenyl)-3-(3-(pyridin-4-yl)-1H-1,2,4-triazol-5-yl)propan-1-one

A 50 mL round bottom flask was charged with 1-(4-methoxyphenyl)-3-(5-(pyridin-4-yl)-2H-1,2,4-triazol-3-yl)propan-1-one (300 mg, 0.97 mmol), and HBr. (30 mL, 40% aq.). The resulting solution was stirred for 4 hours in a 110° C. oil bath. Reaction progress was monitored by TLC (CH₂Cl₂/methanol=10/1). Work-up: the mixture was concentrated, pH adjusted to 7-8 with NaHCO₃ (1N), filtered, concentrated, and purified by flash chromatography (10/1 CH₂Cl₂/methanol). This gave the product, 0.2 g, 65% yield, as a white solid. ¹H NMR (300 MHz, CD₃OD) δ: 8.4(d, 2H), 8.0(d, 2H), 7.7 (d, 2H), 6.6 (d, 2H), 3.3 (m, 2H), 3.0 (m, 2H). LCMS (M+1)⁺: 295.5.

### EXAMPLE 64

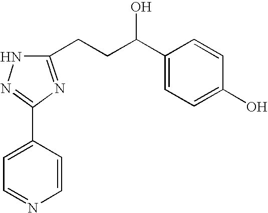

### 4-(1-hydroxy-3-(3-(pyridin-4-yl)-1H-1,2,4-triazol-5-yl)propyl)phenol

A 50 mL round bottom flask was charged with 1-(4-hydroxyphenyl)-3-(5-(pyridin-4-yl)-2H-1,2,4-triazol-3-yl)propan-1-one (100 mg, 0.34 mmol), methanol (20 mL), and cooled to 0° C. NaBH₄ (100 mg, 2.63 mmol) was added portion-wise over 20 min., and stirred for 1 hr at room temperature. Reaction progress was monitored by TLC (CH₂Cl₂/methanol=10/1). Work-up: the reaction was diluted with water (0.1 mL), pH adjusted to 7-8 with NaHCO₃ (60% aq.), filtered, and purified by flash chromatography (10/1 CH₂Cl₂/methanol). This gave 60 mg, 57% yield of product as a white solid. ¹H NMR (300 MHz, CD₃OD) δ: 8.6(d, 2H), 8.0(d, 2H), 7.2 (d, 2H), 6.7 (d, 2H), 4.6 (d, 1H), 2.9 (m, 2H), 2.2 (m, 2H). LCMS (M+1)⁺: 297.3.

### EXAMPLE 65

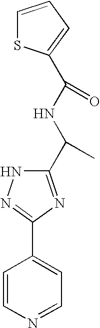

### N-(1-(3-(Pyridin-4-yl)-1H-1,2,4-triazol-5-yl)ethyl)thiophene-2-carboxamide

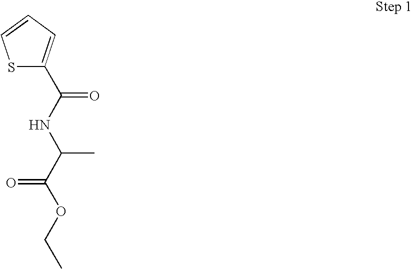

### Ethyl 2-(thiophene-2-carboxamido)propanoate

A 50 mL round bottom flask was charged with DL-alanine ethyl ester hydrochloride (307 mg, 2.00 mmol), thiophene-2-carboxylic acid (256 mg, 2.00 mmol), N-(3-dimethylaminopropyl)-N′-ethylcarbodiimide hydrochloride (383 mg, 2.00 mmol), 1-hydroxybenzotriazole hydrate (31 mg, 0.20 mmol), N,N-diisopropylethylamine (1.05 mL, 6.03 mmol), and CH₂Cl₂ (20 mL). The resulting mixture was stirred overnight at room temperature, diluted with EtOAc (80 mL), washed with water, NaHCO₃ (aq), brine, dried over Na₂SO₄, filtered, and evaporated. The resulting crude product was purified by silica gel chromatography, eluting with EtOAc and hexanes to afford the title compound (354 mg, 78%) as a white solid. ¹H NMR (400 MHz, CDCl₃) δ: 7.55 (m, 1H), 7.48 (m, 1H), 7.08 (m, 1H), 6.61 (bd, 1H), 4.75 (m, 1H), 4.24 (q, 2H), 1.51 (d, 3H), 1.32 (t, 3H).

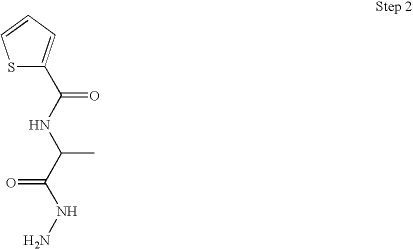

### N-(1-Hydrazinyl-1-oxopropan-2-yl)thiophene-2-carboxamide

A 20 mL screw cap vial was charged with ethyl 2-(thiophene-2-carboxamido)propanoate (344 mg, 1.51 mmol), methanol (1.6 mL), and hydrazine (0.238 mL, 7.58 mmol), then placed in a 70° C. oil bath. The reaction mixture was stirred at that temperature for 1.5 h, then allowed to cool overnight. A solid formed which was collected by filtration and air dried. The filtrate was evaporated and the solid residue was washed with methanol, air dried and combined with the first solid, providing the title compound as a white solid (172 mg, 53%), which was used in the next step without further purification. ¹H NMR (400 MHz, DMSO-d₆) δ: 9.16 (bs, 1H), 8.53 (d, 1H), 7.90 (m, 1H), 7.75 (m, 1H), 7.15 (m, 1H), 4.42 (m, 1H), 4.21 (bd, 2H), 1.30 (d, 3H).

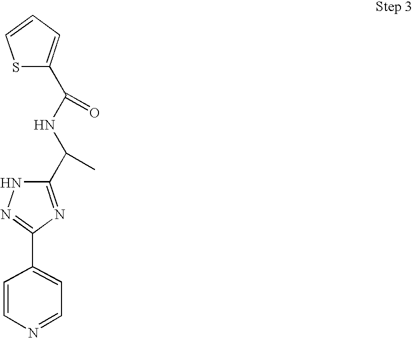

### N-(1-(3-(Pyridin-4-yl)-1H-1,2,4-triazol-5-yl)ethyl)thiophene-2-carboxamide

A 4 mL screw cap vial was charged with N-(1-hydrazinyl-1-oxopropan-2-yl)thiophene-2-carboxamide (49 mg, 0.23 mmol), 4-cyanopyridine (62.5 mg, 0.600 mmol), K₂CO₃ (13.8 mg, 0.0998 mmol), and n-butanol (0.4 mL), then placed in a 120° C. oil bath. The reaction mixture was stirred at that temperature for 16 h, then allowed to cool. A solid formed that was filtered. The filter cake was washed with CH₂Cl₂/methanol 9:1 (2×2 mL). The combined filtrates were evaporated and purified by silica gel chromatography, eluting with 10% methanol and CH₂Cl₂, to afford the title compound (49 mg, 71%) as a faintly yellow film. ¹H NMR (400 MHz, DMSO-d₆) δ: 9.03 (bd, 1H), 8.67 (m, 2H), 7.90 (m, 3H), 7.78 (m, 1H), 7.18 (m, 1H), 4.32 (m, 1H), 1.63 (d, 3H). LCMS (M+1⁺): 300.00.

### EXAMPLE 66

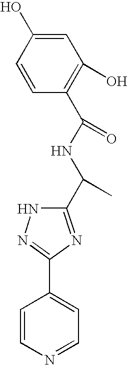

### 2,4-Dihydroxy-N-(1-(3-(pyridin-4-yl)-1H-1,2,4-triazol-5-yl)ethyl)benzamide

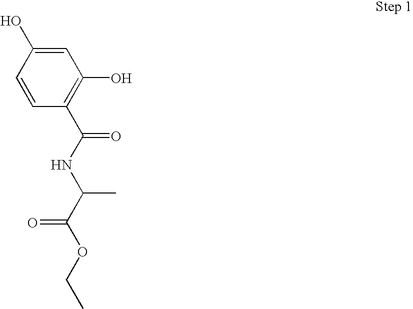

### Ethyl 2-(2,4-dihydroxybenzamido)propanoate

A 100 mL round bottom flask was charged with DL-alanine ethyl ester hydrochloride (768 mg, 5.00 mmol), 2,4-dihydroxybenzoic acid 771 mg, 5.00 mmol), N,N′-diisopropylcarbodiimide (0.775 mL, 5.00 mmol), 1-hydroxybenzotriazole (68 mg, 0.50 mmol), 4-methylmorpholine (1.65 mL, 15.0 mmol), and THF (25 mL). The resulting mixture was stirred overnight at room temperature, then evaporated to dryness. The crude product was purified by silica gel chromatography, eluting with EtOAc in hexanes to afford the title compound (646 mg, 51%) as a white solid contaminated with an unknown impurity. LCMS (M+1⁺): 254.05.

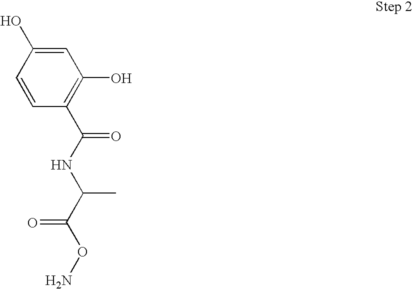

### N-(1-Hydrazinyl-1-oxopropan-2-yl)-2,4-dihydroxybenzamide

A 20 mL screw cap vial was charged with ethyl 2-(2,4-dihydroxybenzamido)propanoate (640 mg, 2.52 mmol), methanol (3 mL), and hydrazine (0.397 mL, 12.6 mmol), then placed in a 70° C. oil bath. The reaction mixture was stirred at that temperature for 2 h, then allowed to cool and evaporated. The residue was taken up in methanol (10 mL), where a solid formed that was collected by filtration and air dried, providing the title compound (596 mg, 99%), which was used in the next step without further purification.

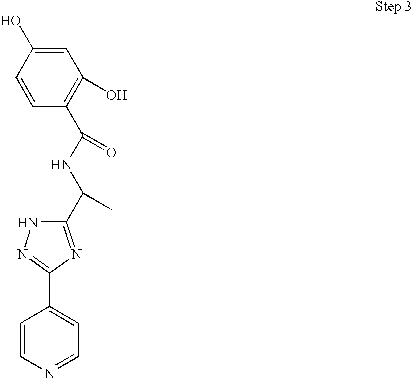

### 2,4-Dihydroxy-N-(1-(3-(pyridin-4-yl)-1H-1,2,4-triazol-5-yl)ethyl)benzamide

A 20 mL screw cap vial was charged with N-(1-hydrazinyl-1-oxopropan-2-yl)-2,4-dihydroxybenzamide (239 mg, 1.00 mmol), 4-cyanopyridine (312 mg, 3.00 mmol), 1,8-diazabicyclo[5.4.0]undec-7-ene (0.150 mL, 1.00 mmol), and n-butanol (3 mL), then placed in a 120° C. oil bath. The mixture was stirred at this temperature for 16 h, then cooled and evaporated. The crude product was purified by silica gel chromatography, eluting with 10% methanol and CH₂Cl₂. The resulting product was further purified by C18 reverse phase semi-preparative HPLC to afford the title compound (mono TFA salt, 39.2 mg, 9%) as a film. ¹H NMR (400 MHz, DMSO-d₆) δ: 12.52 (bs, 1H), 10.13 (bs, 1H), 8.99 (m, 1H), 8.81 (m, 2H), 8.20 (bm, 2H), 7.80 (m, 1H), 6.31 (m, 1H), 6.24 (m, 1H), 5.36 (m, 1H), 1.62 (d, 3H). LCMS (M+1⁺): 326.03.

### EXAMPLE 67

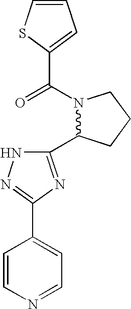

### (2-(3-(pyridin-4-yl)-1H-1,2,4-triazol-5-yl) pyrrolidin-1-yl) (thiophen-2-yl)methanone

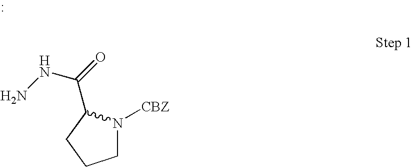

### benzyl 2-(hydrazinecarbonyl)pyrrolidine-1-carboxylate

A 1 liter round bottom flask was charged with 1-(benzyloxycarbonyl)pyrrolidine-2-carboxylic acid (24.9 g, 100 mmol), THF (400 mL), 4-methylmorpholine (12.1 mL, 110 mmol), and ethyl chloroformate (10.5 mL, 110 mmol). The solution was stirred for 2 hours at room temperature, where a white solid precipated. The precipitate was filtered; and the remaining filtrate was added slowly to a cooled solution of hydrazine (15.7 mL, 500 mmol). The reaction was initially stirred at 0° C., and was allowed to reach room temperature over the course of 1 hour. Reaction progress was monitored by TLC (MeOH/methylene chloride=1/10), and visualized with KMnO₄ stain. Workup: the reaction was quenched with water (150 mL), and extracted into EtOAc (200 mL). The organic layer was then washed with citric acid (100 mL, 1M), saturated sodium bicarbonate (100 mL), and brine (50 mL). The organic layers were combined, dried over MgSO₄; and concentrated to give 22.0 g of the product as a viscous oil (88% yield).

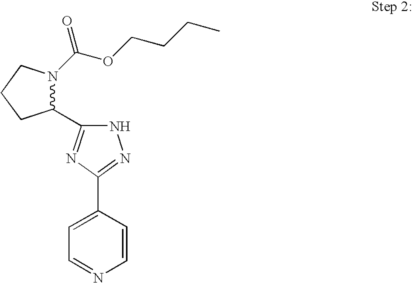

### butyl 2-(3-(pyridin-4-yl)-1H-1,2,4-triazol-5-yl)pyrrolidine-1-carboxylate

A 300 mL sealed tube was charged with benzyl 2-(hydrazinecarbonyl)pyrrolidine-1-carboxylate (2.08 g, 7.9 mmol), isonicotinonitrile (10.51 g, 15.8 mmol), K₂CO₃ (1.09 g, 7.9 mmol), and butan-1-ol (16 mL). The suspension was heated to 160° C., and was allowed to stir overnight. The suspension went into solution at 100° C. The reaction progress was monitored by the disappearance of hydrazide on TLC (MeOH/methylene chloride=1:20). Workup: the mixture was cooled to room temperature; concentrated, and chromatographed on 240 g of silica gel (0 to 4% MeOH/methylene chloride, gradient elution), to give 1.47 g of product as a white solid (59% yield). LCMS (M+1)⁺: 315.99 (M+1)⁺.

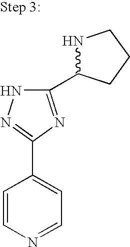

### 4-(5-(pyrrolidin-2-yl)-1H-1,2,4-triazol-3-yl) pyridine

A 50 mL flask was charged with butyl 2-(3-(pyridin-4-yl)-1H-1,2,4-triazol-5-yl)pyrrolidine-1-carboxylate (315 mg, 1.0 mmol), and HBr/acetic acid (5 mL). The reaction was stirred under nitrogen at 65° C. overnight. The reaction was monitored by TLC (MeOH/methylene chloride=1:10) and LCMS to observe the disappearance of starting material. Workup: the reaction mixture was diluted with toluene (10 mL), and concentrated to give the HBr salt of 4-(5-(pyrrolidin-2-yl)-1H-1,2,4-triazol-3-yl)pyridine. The salt was neutralized with NH₃/methanol and recrystallized from methanol to give 173 mg of product as a free base. ¹H NMR (400 MHz, DMSO-d₆): δ 9.89 (bs, 1H), 9.36 (bs, 1H), 9.07 (d, 2H), 8.52 (d, 2H), 5.73 (s, 2H), 3.53 (s, 2H), 3.38 (m, 2H), 1.97 (s, 3H). LCMS (M+1)⁺: 216.10.

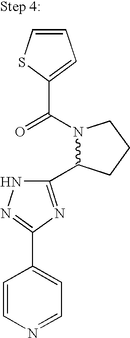

### (2-(3-(pyridin-4-yl)-1H-1,2,4-triazol-5-yl) pyrrolidin-1-yl)(thiophen-2-yl)methanone

An 8 mL vial was charged with 4-(5-(pyrrolidin-2-yl)-1H-1, 2, 4-triazol-3-yl)pyridine (43 mg, 0.2 mmol), thiophene-2-carbonyl chloride (23 μl, 0.21 mmol), triethylamine (28 μl), 0.2 mmol), and methylene chloride (1.0 mL). The reaction was stirred at room temperature for 5 minutes, and progress was monitored by LC/MS. Workup: the resulting mixture gave bis-acylated product, which was converted to the desired mono acyl product by treating with sodium hydroxide (IN aq.), methanol. The solution was concentrated, dissolved in with DMSO, and purified via reverse phase HPLC giving 32 mg of product as a TFA salt (56% yield). ¹H NMR (400 MHz, DMSO-d₆): δ 8.81 (d, 2H), 8.22 (d, 2H), 7.81 (d, 1H), 7.71 (m, 1H), 7.16 (m, 1H), 5.74 (s, 2H), 5.35 (bs, 1H), 4.10 (m, 1H), 3.92 (bs, 2H), 2.38 (m, 1H), 2.09 (m, 1H), 1.98 (m, 1H). LCMS (M+1)⁺: 325.75.

### EXAMPLE 68

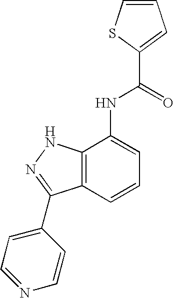

### N-(3-(Pyridin-4-yl)-1H-indazol-7-yl)thiophene-2-carboxamide

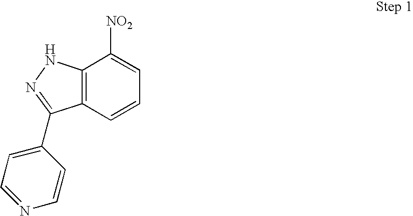

### 7-Nitro-3-(pyridin-4-yl)-1H-indazole

To a solution of 3-bromo-7-nitro-1H-indazole (0.25g, 1 mmol), and pyridin-4-ylboronic acid (0.14 g, 1 mmol) in 3:1 THF/water (4 mL), was added an aqueous solution of Na₂CO₃ (1.25 mL, 2M). The mixture was vacuum flushed with nitrogen (3×), treated with Pd(Ph₃P)₂Cl₂ (0.070 g, 0.1 mmol) in one portion, and heated to 70° C. for 6 h. LCMS confirmed the completion of the reaction. The reaction mixture was diluted with ethyl acetate (100 mL), extracted with water, brine, dried over Na₂SO₄, filtered, and concentrated. The mixture was purified by silica gel column chromatography (0-50% ethyl acetate in hexanes) to give the product as a yellow solid (0.2g, 80% yield). LCMS (M+1⁺): 243.87.

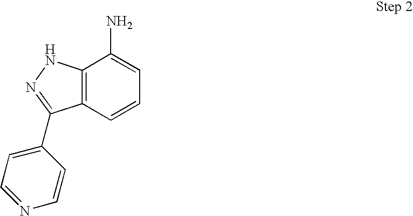

### 3-(Pyridin-4-yl)-1H-indazol-7-amine

To a solution of 7-nitro-3-(pyridin-4-yl)-1H-indazole (0.15g, 0.62 mmol) in ethyl acetate (6.2 mL) was added Pd/C (0.066g, 10% Degussa type). The mixture was hydrogenated at 40 psi, at room temperature for 2 h. Reaction completion was confirmed by LCMS. Work-up: the mixture was filtered through celite, and concentrated in vacuo to give the title compound (0.12g, 92% yield) as light brown solid. LCMS: (M+1)⁺: 211.95.

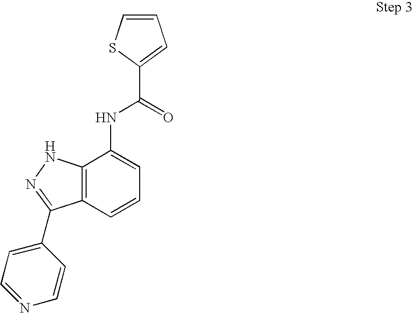

### N-(3-(Pyridin-4-yl)-1H-indazol-7-yl)thiophene-2-carboxamide

To a solution of 3-(pyridin-4-yl)-1H-indazol-7-amine (0.05g, 0.24 mmol) in DMF (1.2 mL), was added thiophene-2-carboxylic acid (0.03 g, 0.24 mmol), triethyl amine (0.048 g, 0.48 mmol) followed by HATU (0.091 g, 0.24 mmol). The resulting mixture was stirred at room temperature overnight. Reaction progress was monitored by LCMS. The reaction was diluted with water, extracted with ethyl acetate (3×25 mL), brine, dried over Na₂SO₄, filtered, and concentrated. The crude product was purified by C18 reversed phase HPLC, affording the title compound (40 mg, 53% yield) as yellow solid. ¹H NMR (400 MHz, DMSO-d6): δ 8.26(dd, 1H), 8.17(dd, 1H), 7.31-7.25 (m, 4H), 6.96 (d, 2H), 6.86 (d, 2H); LCMS: (M+1)⁺: 321.74.

### EXAMPLE 69

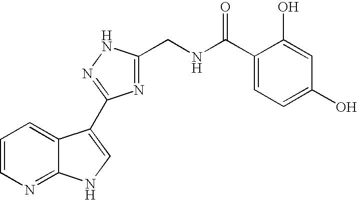

### N-((3-(1H-pyrrolo[2, 3-b]pyridin-3-yl)-1H-1,2,4-triazol-5-yl) methyl)thiophene-2-carboxamide

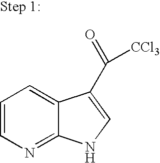

### 2,2,2-trichloro-1-(1H-pyrrolo[2,3-b]pyridin-3-yl)ethanone

A 500 mL flask was charged with aluminum trichloride (13.6 g, 102 mmol), methylene chloride (108 mL), and was stirred at −78° C. To this was added a solution of trichloroacetyl chloride (10.8 mL, 96.2 mmol) and methylene chloride (54 mL). The reaction was allowed to stir for 20 minutes at −78° C., then gradually warmed to −40° C., where a solution of 1H-pyrrolo[2,3-b]pyridine (4 g, 33.9 mmol) in methylene chloride (20 mL) was added. The solution was allowed to stir for 20 minutes at this temperature, then brought to 0° C. by placing in an ice bath, where the reaction was further stirred for 1 hour. Workup: the mixture was diluted with 1M HCl (55 mL), and water (50 mL). The organic layer was separated and washed with of sat. NaHCO₃, dried over MgSO₄, filtered, and concentrated, giving the product as a white solid, 4.38 g (50% yield).

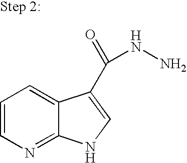

### 1H-pyrrolo[2,3-b]pyridine-3-carbohydrazide

A 250 mL flask was charged with 2, 2, 2-trichloro-1-(1H-pyrrolo[2, 3-b]pyridin-3-yl) ethanone (5.0 mg, 19.0 mmol), DMF (60.0 mL), and hydrazine (18 mL, 570 mmol). The reaction was stirred at room temperature for 15 minutes, or at which time disappearance of starting material was observed by TLC (MeOH/methylene chloride=1:5). Workup: the reaction was concentrated and dried to give 3.19 g of product as a white powder (50% yield). ¹H NMR (400 MHz, DMSO-d₆): δ 12.02 (bs, 1H), 9.26 (s, 1H), 8.42 (d, 1H), 8.24 (d, 1H), 8.06 (s, 1H), 7.15 (q, 1H), 4.32 (s, 2H). LCMS (M+1)⁺: 176.99.

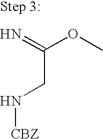

### methyl 2-(benzyloxycarbonylamino)acetimidate

A 100 mL flask was charged with benzyl cyanomethylcarbamate (0.95 g, 5.0 mmol), MeOH (0.3 mL, 7.5 mmol), and 4M HCl in dioxane (10 mL). The reaction was stirred at room temperature for 2 hours, whereupon a solid precipitate formed. Workup: the precipitate was sonicated; filtered; washed with ether; and dried to give 0.90 g of white solid product (81% yield).

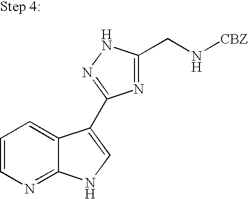

### benzyl (3-(1H-pyrrolo[2,3-b]pyridin-3-yl)-1H-1,2,4-triazol-5-yl)methylcarbamate

A 250 mL round bottom flask was charged with methyl 2-(benzyloxycarbonylamino)acetimidate (3.58 g, 16.1 mmol), 1H-pyrrolo[2, 3-b]pyridine-3-carbohydrazide (1.42 g, 8.1 mmol), and methanol (60 mL). The reaction was stirred at 50° C. for 30 minutes, at which timeall strating material had been consumed by TLC (MeOH/ methylene chloride=8:100). Workup: the reaction was concentrated; diluted with DMSO (2 mL); and purified via reverse phase HPLC to give 900 mg of product as a white powder (19% yield).

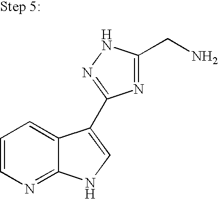

### (3-(1H-pyrrolo[2, 3-b]pyridin-3-yl)-1H-1, 2, 4-triazol-5-yl) methanamine

A 250 mL round bottom flask was flushed with nitrogen; then charged with benzyl(3-(1H-pyrrolo[2,3-b]pyridin-3-yl)-1H-1,2,4-triazol-5-yl)methylcarbamate (348 mg, 1.0 mmol), Pd/C (74 mg, 20% wt), and ethanol (1 mL). the reaction was flushed with hydrogen (3×), then stirred overnight. Work-up: the reaction was filtered and concentrated, to giving the product as a solid 210 mg (quantitative yield).

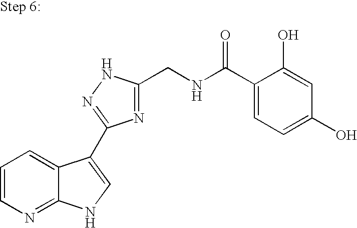

### N-((3-(1H-pyrrolo[2, 3-b]pyridin-3-yl)-1H-1,2,4-triazol-5-yl) methyl)thiophene-2-carboxamide

A 4 mL vial was charged with (3-(1H-pyrrolo[2, 3-b]pyridin-3-yl)-1H-1,2,4-triazol-5-yl) methanamine (75 mg, 0.347 mmol), 2,4-dihydroxybenzoic acid (54 mg, 0.52 mmol), DMF (1.74 mL), HOBT (26 mg, 0.017 mmol), and DCC (72 mg, 0.416 mmol). The solution was stirred at room temperature until starting material was consumed and observed on TLC (MeOH/methylene chloride=1/10). Workup: the reaction was diluted with 0.5 mL of DMSO; purified via reverse phase HPLC; and concentrated to give 20 mg of a white powder, as product (17% yield). ¹H NMR (400 MHz, DMSO-d₆): δ 12.56 (bs, 1H), 12.37 (bs, 1H), 10.14 (bs, 1H), 9.22 (bs, 1H), 8.37 (m, 2H), 8.26 (s, 1H), 7.72 (d, 1H), 7.27 (q, 1H), 6.32 (dd, 1H), 6.25 (s, 1H), 4.76 (d, 2H). LCMS (M+1)⁺: 351.90.

### EXAMPLE 70

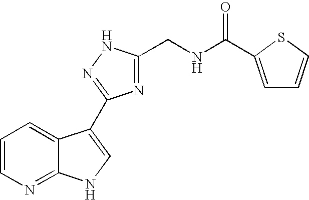

### N-((3-(1H-pyrrolo[2,3-b]pyridin-3-yl)-1H-1,2,4-triazol-5-yl)methyl)thiophene-2-carboxamide

The title compound was prepared analogously to Example 69, where thiophene 2-carboxylic acid was substituted for 2,4-dihydroxybenzoic acid in the final step of that sequence. ¹H NMR (400 MHz, DMSO-d₆): δ 12.59 (s, 1H), 9.92 (bs, 1H), 8.79 (d, 2H), 8.39 (m, 2H), 8.27 (s, 1H), 7.35 (m, 2H), 7.27 (q, 1H), 4.67 (d, 2H). LCMS (M+1)⁺: 325.98.

### EXAMPLE 71

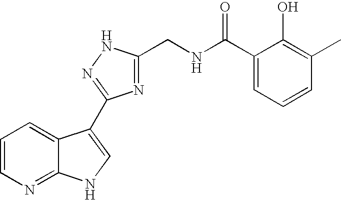

### N-((3-(1H-pyrrolo[2,3-b]pyridin-3-yl)-1H-1,2,4-triazol-5-yl)methyl)-2-hydroxy-3-methylbenzamide

The title compound was prepared analogously to Example 69, where 2-hydroxy-3-methylbenzoic acid was substituted for 2,4-dihydroxybenzoic acid in the final step of that sequence. ¹H NMR (400 MHz, DMSO-d₆): δ 12.77 (s, 1H), 12.57 (bs, 1H), 9.63 (bs, 1H), 8.38 (m, 2H), 8.27 (s, 1H), 7.73 (d, 1H), 7.33 (d, 1H), 7.27 (m, 1H), 6.82 (m, 1H), 4.80 (d, 2H), 2.15 (s, 3H). LCMS (M+1)⁺: 349.98.

### EXAMPLE 72

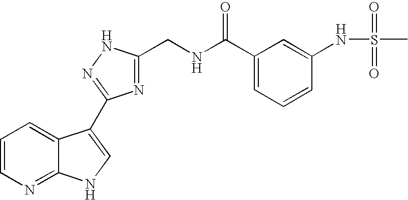

### N-((3-(1H-pyrrolo[2,3-b]pyridin-3-yl)-1H-1,2,4-triazol-5-yl)methyl)-3-(methylsulfonamido)benzamide

The title compound was prepared analogously to Example 69, where 3-(methylsulfonamido)benzoic acid was substituted for 2, 4-dihydroxybenzoic acid in the final step of that sequence. ¹H NMR (400 MHz, DMSO-d₆): δ 12.57 (s, 1H), 9.92 (bs, 1H), 9.30 (m, 1H), 8.37 (m, 2H), 8.26 (s, 1H), 7.72 (d, 1H), 7.62 (d, 1H), 7.45 (m, 1H), 7.35 (m, 2H), 7.27 (q, 1H), 4.76 (d, 2H), 2.15 (s, 3H). LCMS (M+1)⁺: 412.74.

### EXAMPLE 73

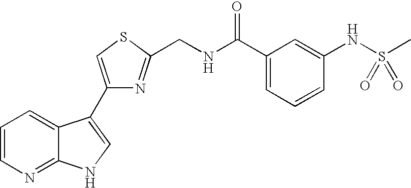

### N-((4-(1H-pyrrolo[2,3-b]pyridin-3-yl)thiazol-2-yl)methyl)-3-(methylsulfonamido)benzamide

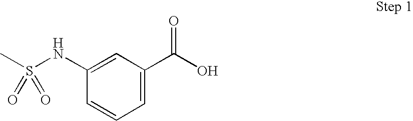

### 3-(Methylsulfonamido)benzoic acid

A round bottom flask was charged with a solution of 3-aminobenzoic acid (3.00 g, 21.9 mmol), and methylene chloride (100 mL), then cooled to 0° C. in an ice/water bath. To the solution was added pyridine (5.3 mL, 65.6 mmol), followed by methanesulfonyl chloride (2.0 mL, 26.3 mmol). The resulting solution was allowed to react for 1 hour while the temperature was maintained at 0° C. The ice/water bath was removed and the solution was allowed to warm up to room temperature over 2 hours. Work-up: the solution was diluted with EtOAc (200 mL), washed with 1N HCl (300 mL), saturated sodium chloride (300 mL), dried over Na₂SO₄, and concentrated by rotary evaporator. The resulting pink solid was purified by column chromatography (silica gel, 50-100% EtOAc/hexanes), resulting in 2.29 g (49% yield) of product as an off-white solid.

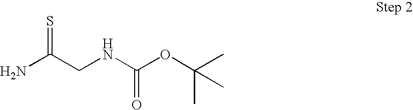

### Tert-butyl 2-amino-2-thioxoethylcarbamate

A round bottom flask was charged with tert-butyl cyanomethylcarbamate (10.0 g, 64 mmol), triethylamine (20 mL), and pyridine (80 mL). The mixture was vacuum flushed with hydrogen sulfide in a balloon. The resulting mixture was allowed to react for 23 hours at room temperature. Excess hydrogen sulfide was removed by bubbling nitrogen gas through the reaction mixture. The mixture was concentrated via rotary evaporator. The black solid thus obtained was recrystalized from boiling EtOAc/hexanes to give 9.80 g (80% yield) of a tan solid.

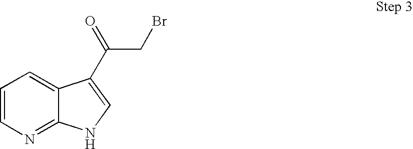

### 2-Bromo-1-(1H-pyrrolo[2,3-b]pyridin-3-yl)ethanone

A round bottom flask was charged with 1H-pyrrolo[2,3-b]pyridine (5.93 g, 50.2 mmol), and anhydrous aluminum chloride (13.39 g, 100.4 mmol). To the above solution was added 2-bromoacetyl bromide (6.54 mL, 75.3 mmol) over a period of 5 minutes. The resulting mixture was stirred for 45 minutes at room temperature, and then heated to 50° C. for 1 hour. Work-up: the mixture was poured into water (1000 mL) and extracted two times with methylene chloride (500 mL). The aqueous layer was filtered, the pH was adjusted to 12 via addition of 2N NaOH, and was then extracted two times with EtOAc (500 mL). The methylene chloride and EtOAc extracts were combined, washed with 1000 mL of brine, dried over Na₂SO₄, and concentrated via rotary evaporator. The brown solid obtained was purified by flash chromatography (silica gel, 0-100% EtOAc/hexanes), resulting in 1.404 g (12% yield) of product as a white solid.

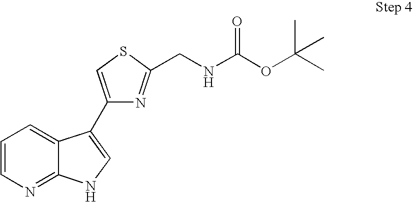

### Tert-butyl(4-(1H-pyrrolo[2,3-b]pyridin-3-yl)thiazol-2-yl)methylcarbamate

A round bottom flask was charged with 2-Bromo-1-(1H-pyrrolo[2,3-b]pyridin-3-yl)ethanone (0.70 g, 2.9 mmol), tert-butyl 2-amino-2-thioxoethylcarbamate (0.67 g, 3.5 mmol), and EtOH (15 mL). The resulting solution was heated to 70° C. for 30 minutes while stirring. Work-up: the mixture was diluted with 150 mL of EtOAc, washed with saturated NaCl (150 mL), dried over Na₂SO₄, concentrated via rotary evaporator, and purified via column chromatography (silica gel, 30-100% EtOAc/hexanes), resulting in 0.521 g (54% yield) of product as a white solid.

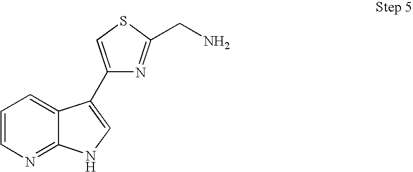

### (4-(1H-Pyrrolo[2,3-b]pyridin-3-yl)thiazol-2-yl)methanamine

A round bottom flask was charged with tert-butyl (4-(1H-pyrrolo[2,3-b]pyridin-3-yl)thiazol-2-yl)methylcarbamate (0.521 g, 1.58 mmol), trifluoroacetic acid (4 mL), and methylene chloride (12 mL). The resulting solution was stirred at room temperature for 30 minutes. Work-up: the reaction was diluted with toluene (10 mL) and concentrated via rotary evaporator resulting in 0.300 g (83% yield) of product as a white solid.

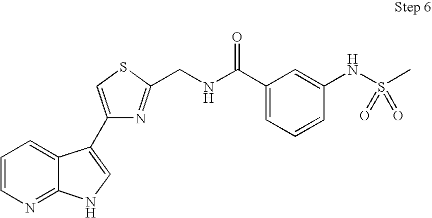

### N-((4-(1H-pyrrolo[2,3-b]pyridin-3-yl)thiazol-2-yl)methyl)-3-(methylsulfonamido)benzamide

A round bottom flask was charged with (4-(1H-pyrrolo[2,3-b]pyridin-3-yl)thiazol-2-yl)methanamine (0.100 g, 0.43 mmol), 3-(methylsulfonamido)benzoic acid (0.112 g, 0.52), 2-(1H-benzo[d][1,2,3]triazol-1-yl)-1,1,3,3-tetramethylisouronium hexafluorophosphate(V) (“HBTU”) (0.214 g, 0.56 mmol), triethylamine (0.12 mL, 0.86 mmol), and DMF (5 mL). The resulting solution was stirred overnight at room temperature. Work-up: the mixture was concentrated and purified via semi-preparative reverse phase HPLC, resulting in 0.134 g (72% yield) of product as a yellow solid. ¹H NMR (400 MHz, DMSO-d₆) δ: 9.96 (s, 1H), 9.48 (t, 1H), 8.56 (d, 1H), 8.30 (d, 1H), 8.00 (s, 1H), 7.79 (s, 1H), 7.75 (s, 1H), 7.65 (d, 1H), 7.48 (t, 1H), 7.39 (d, 1H), 7.20 (a-b quartet, 1H), 4.82 (d, 2H), 3.04 (s, 3H). LCMS (M+1⁺): 427.72

### EXAMPLE 74

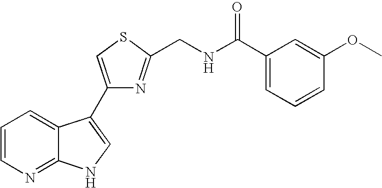

### N-((4-(1H-pyrrolo[2,3-b]pyridin-3-yl)thiazol-2-yl)methyl)-3-methoxybenzamide

A round bottom flask was charged with (4-(1H-pyrrolo[2,3-b]pyridin-3-yl)thiazol-2-yl)methanamine (0.100 g, 0.43 mmol, prepared as described in step 5 of example 73), and triethylamine (0.12 mL, 0.86 mmol), and methylene chloride (5 mL). The resulting solution was cooled to 0° C. in an ice/water bath. To the cooled solution was added 3-methoxybenzoyl chloride (0.079 mL, 0.56 mmol). The resulting solution was allowed to warm to room temperature and stirred overnight. Work-up: the mixture was concentrated and purified via semi-preparative reverse phase HPLC, resulting in 0.136 g (86% yield) of product as a yellow solid. ¹H NMR (400 MHz, DMSO-d₆) δ: 11.96 (s, 1H), 9.46 (t, 1H), 8.53 (d, 1H), 8.29 (d, 1H), 7.98 (s, 1H), 7.77 (s, 1H), 7.51 (d, 1H), 7.48 (s, 1H), 7.43 (t, 1H), 7.18 (a-b quartet, 1H), 7.14 (d, 1H), 4.82 (d, 2H), 3.82 (s, 3H). LCMS (M+1⁺): 364.84

### EXAMPLE 75

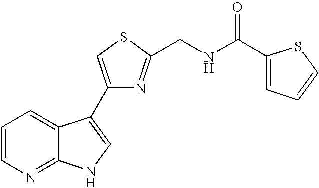

### N-((4-(1H-pyrrolo [2,3-b]pyridin-3-yl)thiazol-2-yl)methyl)thiophene-2-carboxamide

The title compound was prepared analogously to N-((4-(1H-pyrrolo[2,3-b]pyridin-3-yl)thiazol-2-yl)methyl)-3-methoxybenzamide (Example 75), where thiophene-2-carbonyl chloride (0.06 mL, 0.56 mmol) was substituted for 3-methoxybenzoyl chloride. ¹H NMR (400 MHz, DMSO-d₆) δ: 11.95 (s, 1H), 9.47 (t, 1H), 8.52 (d, 1H), 8.28 (d, 1H), 7.98 (s, 1H), 7.86 (d, 1H), 7.82 (d, 1H), 7.78 (s, 1H), 7.19 (bs, 2H), 4.80 (d, 2H). LCMS (M+1⁺): 340.80

### EXAMPLE 76

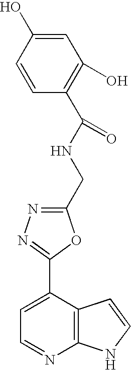

### N-((5-(1H-Pyrrolo[2,3-b]pyridin-4-yl)-1,3,4-oxadiazol-2-yl)methyl)-2,4-dihydroxybenzamide

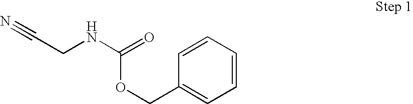

### Benzyl cyanomethylcarbamate

A 2L round bottom flask was charged with 2-aminoacetonitrile hydrochloride (50 g, 540 mmol), THF (300 mL), potassium carbonate (224 g, 1.62 mol), and water (300 mL), then cooled to 0° C. To this was added a solution of benzyl chloroformate (91 g, 540 mmol) in THF (100 mL), dropwise over 30 min. The resulting solution was allowed to react for 3 h at room temperature. Reaction progress was monitored by TLC (50% EtOAc in petroleum ether). Work-up: the solution was extracted with EtOAc (200 mL), dried over MgSO₄, and concentrated, giving the title compound (100 g, 92%) as a white solid.

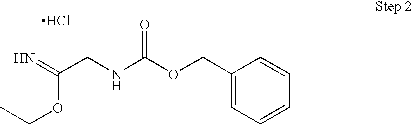

### Ethyl 2-(benzyloxycarbonylamino)acetimidate hydrochloride

A 1L round bottom flask was charged with benzyl cyanomethylcarbamate (50 g, 260 mmol), Et₂O (300 mL), and EtOH (500 mL). After cooling in an ice/salt bath, hydrogen chloride was bubbled through the reaction mixture for 2 h, then the reaction mixture was stirred for an additional 4 h. Work-up: the product was precipitated by the addition of Et₂O (300 mL) at −5° C. and collected by filtration. The filter cake was washed with Et₂O (2×200 mL) giving the title compound (30 g, 42%) as a white solid which was used in the next step without further purification.

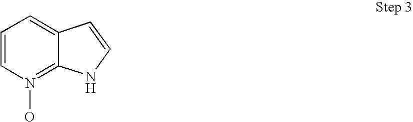

### 1H-Pyrrolo[2,3-b]pyridine 7-oxide

A 3 L round bottom flask was charged with 1H-pyrrolo[2,3-b]pyridine (50 g, 420 mmol), DME (500 mL), and heptane (1 L). m-Chloroperbenzoic acid (75 g, 440 mmol) was added carefully in several batches while maintaining a temperature of 8-26° C. with an ice bath. The resulting solution was allowed to warm to room temperature and stirred for 2.5 h. Reaction progress was monitored by TLC (10% methanol and CH₂Cl₂). Work up: the reaction mixture was filtered and the filter cake was dissolved in 700 mL of water. Aqueous K₂CO₃ (50%) was added to adjust the pH to 9 and the resulting mixture was stirred an additional hour, then cooled in an ice bath and stirred one hour more. Work-up: the solid material was collected by filtration and washed with cold water (3×100 mL) providing the title compound (48 g, 85%) as a white solid.

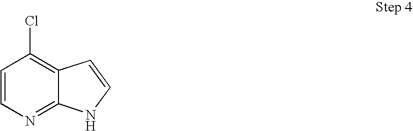

### 4-Chloro-1H-pyrrolo[2,3-b]pyridine

A 500 mL round bottom flask was charged with 1H-pyrrolo[2,3-b]pyridine 7-oxide (12 g, 90 mmol) and DMF (200 mL). Methanesulfonyl chloride (11 g, 97 mmol) was added dropwise at 0° C. over 15 minutes. After 1 h, the cooling bath was removed and the reaction mixture was heated to 50° C. in an oil bath for 15 h. Reaction progress was monitored by TLC (10% methanol in CH₂Cl₂). Work-up: ice water (300 g) was added and the pH was adjusted to 9 with NaOH (5 M, aq.). The mixture was extracted with EtOAc (4×200 mL). The combined organic phases were dried over MgSO4 and evaporated, providing the title compound (10 g, 80%) as a white solid, which was used in the next step without further purification.

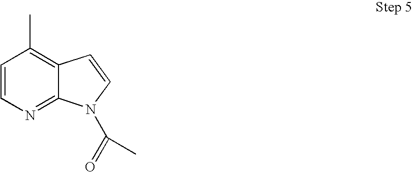

### 1-(4-Iodo-1H-pyrrolo[2,3-b]pyridin-1-yl)ethanone

A 500 mL round bottom flask was charged with 4-chloro-1H-pyrrolo[2,3-b]pyridine (15.2 g, 100 mmol), sodium iodide (76.6 g, 511 mmol), and acetonitrile (200 mL). Acetyl chloride (20 mL) was added dropwise over 40 minutes, and then the reaction mixture was warmed in a 90° C. oil bath for 15 h. Reaction progress was monitored by TLC (20% EtOAc in petroleum ether). Work-up: water was added at 0° C. and the resulting mixture was extracted with EtOAc (4×200 mL). The combined organic layers were dried over Na₂SO₄ and evaporated. The crude product was purified by silica gel chromatography, eluting with EtOAc and petroleum ether to afford the title compound (16 g, 51%) as a white solid.

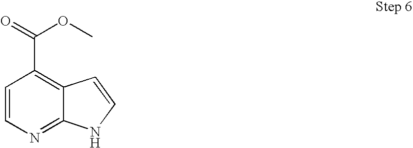

### Methyl 1H-pyrrolo[2,3-b]pyridine-4-carboxylate

A 250 mL round bottom flask was charged with 1-(4-iodo-1H-pyrrolo[2,3-b]pyridin-1-yl)ethanone (3.7 g, 14 mmol), triethylamine (2.6 g, 26 mmol), tetrakis(triphenylphosphine)palladium (2.96 g, 2.56 mmol), and methanol (150 mL). While warming to 60° C. in an oil bath, carbon monoxide (5 g, 200 mmol) was bubbled through the reaction mixture. After 15 h at this temperature, TLC analysis (25% EtOAc in petroleum ether) showed the reaction was complete. Work-up: the mixture was filtered, concentrated, and purified by silica gel chromatography, eluting with EtOAc in petroleum ether to afford the title compound (1.2 g, 50%) as a white solid.

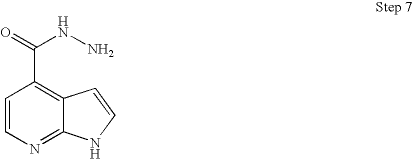

### 1H-Pyrrolo[2,3-b]pyridine-4-carbohydrazide

A 50 mL round bottom flask was charged with methyl 1H-pyrrolo[2,3-b]pyridine-4-carboxylate (1 g, 6 mmol), hydrazine hydrate (85%, 2.85 g, 48.4 mmol), and EtOH (25 mL). The reaction mixture was heated in a 90° C. oil bath for 2 hrs. Reaction progress was monitored by TLC (10% methanol in CH₂Cl₂). Work-up: The mixture was evaporated to dryness providing the title compound (0.5 g, 45%) as a white solid.

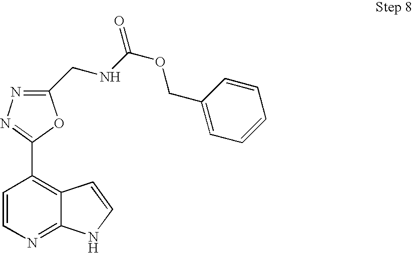

### Benzyl (5-(1H-pyrrolo[2,3-b]pyridin-4-yl)-1,3,4-oxadiazol-2-yl)methylcarbamate

A 50 mL round bottom flask was charged with 1H-pyrrolo[2,3-b]pyridine-4-carbohydrazide (500 mg, 2.84 mmol), ethyl 2-(benzyloxycarbonyl)acetimidate hydrochloride (3.08 g, 11.3 mmol, prepared in Step 2), and EtOH (25 mL), then placed in an 80° C. oil bath and stirred for 1 h. Reaction progress was monitored by TLC (33% EtOAc in petroleum ether). Work-up: the mixture was evaporated and purified by silica gel chromatography, eluting with 50% EtOAc in petroleum ether to afford the title compound (0.8 g, 80%) as a white solid. ¹H NMR (300 MHz, DMSO-d₆) δ: 12.15 (bs, 1H), 8.42 (m, 1H), 8.17 (m, 1H), 7.74 (m, 1H), 7.60 (m, 1H), 7.35 (m, 5H), 6.96 (m, 1H), 5.09 (s, 2H), 4.62 (d, 2H). LCMS (M+1⁺): 350.

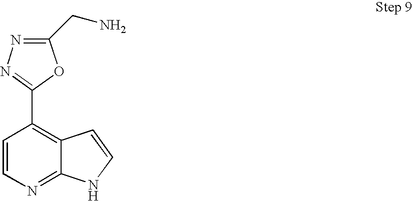

### (5-(1H-Pyrrolo[2,3-b]pyridin-4-yl)-1,3,4-oxadiazol-2-yl)methanamine

A 100 mL round bottom flask was charged with benzyl (5-(1H-pyrrolo[2,3-b]pyridin-4-yl)-1,3,4-oxadiazol-2-yl)methylcarbamate (699 mg, 2.00 mmol), and EtOH (20 mL). Nitrogen was bubbled through the reaction mixture, then Pd/C (10%, Degussa type, 200 mg) was added. The flask was flushed with hydrogen, and stirred for 16h. Nitrogen was bubbled through the reaction mixture, then it was filtered. The filtrate was evaporated giving the title compound. ¹H NMR (300 MHz, DMSO-d₆) δ: 12.14 (bs, 1H), 8.42 (m, 1H), 7.74 (m, 1H), 7.65 (m, 1H), 7.03 (m, 1H), 4.03 (s, 2H).

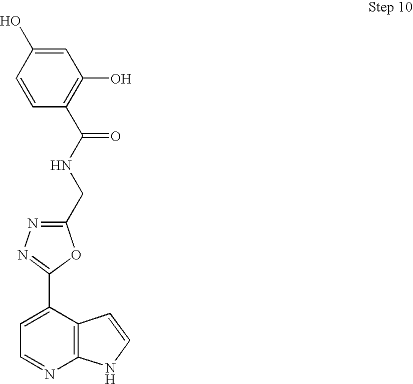

### N-((5-(1H-pyrrolo[2,3-b]pyridin-4-yl)-1,3,4-oxadiazol-2-yl)methyl)-2,4-dihydroxybenzamide

An 8 mL screw cap vial was charged with (5-(1H-pyrrolo[2,3-b]pyridin-4-yl)-1,3,4-oxadiazol-2-yl)methanamine (43 mg, 0.20 mmol), 2,4-dihydroxybenzoic acid (31 mg, 0.20 mmol), 4-methylmorpholine (0.088 mL, 0.80 mmol), N,N′-dicyclohexycarbodiimide (41 mg, 0.20 mmol), 1-hydroxybenzotriazole hydrate (2 mg, 0.01 mmol), DMF (0.5 mL), and acetonitrile (0.5 mL). The resulting mixture was stirred for 16 h, filtered and evaporated. The crude product was purified by C18 reverse phase semi-preparative HPLC, giving the product as a faintly green film (24 mg, 34%.) ¹H NMR (400 MHz, CD₃OD) δ: 8.39 (m, 1H), 7.79 (m, 1H), 7.66 (m, 2H), 7.13 (m, 1H), 6.36 (m, 1H), 6.31 (m, 1H), 4.94 (s, 2H). LCMS (M+1⁺): 352.96

### EXAMPLE 77

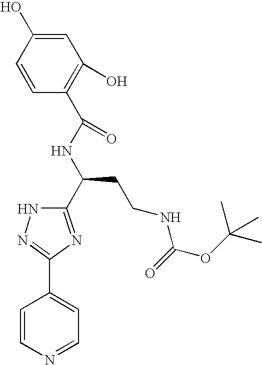

### (S)-tert-Butyl 3-(2,4-dihydroxybenzamido)-3-(3-(pyridin-4-yl)-1H-1,2,4-triazol-5-yl)propylcarbamate

The title compound was prepared analogously to Example 15, where (S)-ethyl 2-amino-4-(tert-butoxycarbonylamino)butanoate and 2,4-bis(benzyloxy)benzoic acid were substituted for ethyl 2-aminoacetate hydrochloride and 2,4-dihydroxybenzoic acid respectively in step 1 of that sequence and the final required debenzylation was carried out under typical hydrogenation conditions as described in step 2 of Example 37. ¹H NMR (400 MHz, DMSO-d₆): δ 14.21 (bs, 1H), 12.46 (bs, 1H), 10.07 (bs, 1H), 8.90 (bm, 1H), 8.65 (m, 2H), 7.90 (m, 2H), 7.82 (m,1H), 6.85 (m, 1H), 6.32 (m, 1H), 6.26 (m, 1H), 5.29 (m, 1H) 3.05 (m, 2H), 2.17 (bm, 2H), 1.36 (s, 9H). LCMS (M+1)⁺: 455.23.

### EXAMPLE 78

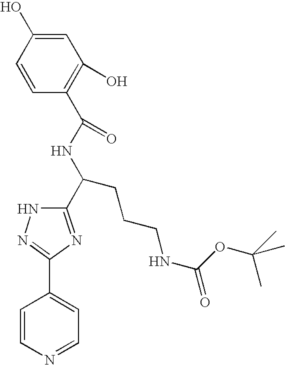

### tert-Butyl 4-(2,4-dihydroxybenzamido)-4-(3-(pyridin-4-yl)-1H-1,2,4-triazol-5-yl)butylcarbamate

The title compound was prepared analogously to Example 15, where ethyl 2-amino-5-(tert-butoxycarbonylamino)pentanoate and 2,4-bis(benzyloxy)benzoic acid were substituted for ethyl 2-aminoacetate hydrochloride and 2,4-dihydroxybenzoic acid respectively in step 1 of that sequence and the final required debenzylation was carried out under typical hydrogenation conditions as described in step 2 of Example 37. ¹H NMR (400 MHz, DMSO-d₆): δ 14.24 (s, 1H), 12.49 (s, 1H), 10.10 (s, 1H), 8.92 (m, 1H), 8.65 (m, 2H), 7.91 (m, 2H), 7.83 (m,1H), 6.82 (m, 1H), 6.32 (m, 1H), 6.26 (m, 1H), 5.25 (m, 1H) 2.97 (m, 2H), 2.01 (bm, 2H), 1.45 (m, 2H) 1.35 (s, 9H). LCMS (M+1)⁺: 469.26.

### EXAMPLE 79

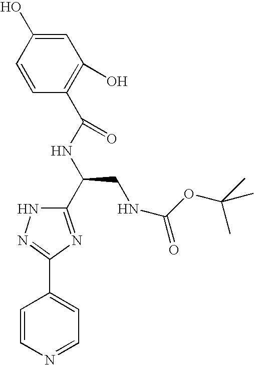

### (S)-tert-Butyl 2-(2,4-dihydroxybenzamido)-2-(3-(pyridin-4-yl)-1H-1,2,4-triazol-5-yl)ethylcarbamate

The title compound was prepared analogously to Example 15, where (S)-ethyl 2-amino-3-(tert-butoxycarbonylamino)propanoate and 2,4-bis(benzyloxy)benzoic acid were substituted for ethyl 2-aminoacetate hydrochloride and 2, 4-dihydroxybenzoic acid respectively in step 1 of that sequence and the final required debenzylation was carried out under typical hydrogenation conditions as described in step 2 of Example 37. ¹H NMR (400 MHz, DMSO-d₆): δ 14.25 (bs, 1H), 12.35 (bs, 1H), 10.08 (bs, 1H), 8.81 (bm, 1H), 8.64 (m, 2H), 7.89 (m, 2H), 7.74 (m,1H), 7.11 (m, 1H), 6.31 (m, 1H), 6.24 (m, 1H), 5.33 (m, 1H) 3.62 (m, 2H), 1.30 (s, 9H). LCMS (M+1)⁺: 441.24.

### EXAMPLE 80

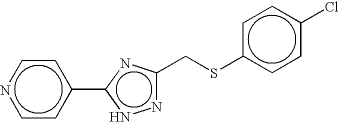

Example 80 is commercially available.

### EXAMPLE 81

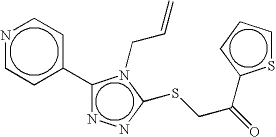

Example 81 is commercially available.

### Compounds Prepared by Parallel Synthesis

The invention is illustrated by the following Schemes:

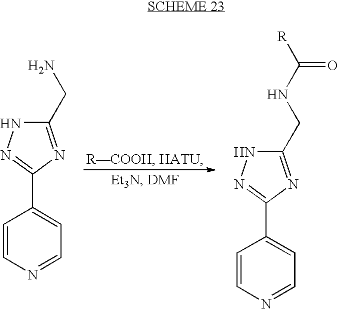

Examples 80-299 can be synthesized using the following general synthetic procedure set forth in Scheme 23.

Starting core: (3-(pyridin-4-yl)-1H-1,2,4-triazol-5-yl)methanamine was prepared as described in Example 16. Where R—COOH is a carboxylic acid selected to afford Examples 77-296, which were prepared by General Procedure 1.

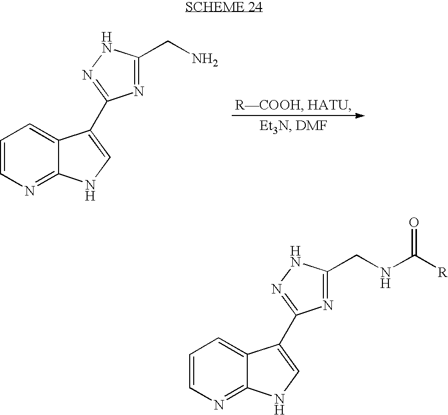

Examples 300-531 can be synthesized using the following general synthetic procedure set forth in Scheme 24.

Starting core: (3-(1H-pyrrolo[2,3-b]pyridin-3-yl)-1H-1,2,4-triazol-5-yl)methanamine was prepared as described in Example 69. Where R—COOH is a carboxylic acid selected to afford Examples 91-324, which were prepared by General Procedure 2.

General Conditions:

General Conditions 1:

Carboxylic acid monomers (4 μmol) in DMF (8 μL) were transferred to each well of 384 well plate, then treated with a solution of core (2.6 μmol) and Et₃N (10.6 μmol) in DMF (16 μL), followed by a solution HATU (3.5 μmol) in DMF (16 μL). The reaction plate was heat sealed and shaken at room temperature for 16 hours. Solvent was removed under vacuum. Products were analyzed for purity by LCMS before testing.

General Conditions 2:

Carboxylic acid monomers (4 μmol) in DMF (8 μL) were transferred to each well of 384 well plate, then treated with a solution of core (2.0 μmol) and Et₃N (2.4 μmol) in DMF (16 μL), followed by a solution HATU (2.2 μmol) in DMF (16 μL). The reaction plate was heat sealed and shaken at room temperature for 16 hours. Solvent was removed under vacuum. Products were analyzed for purity by LCMS before testing.

The invention is further illustrated by the following examples.

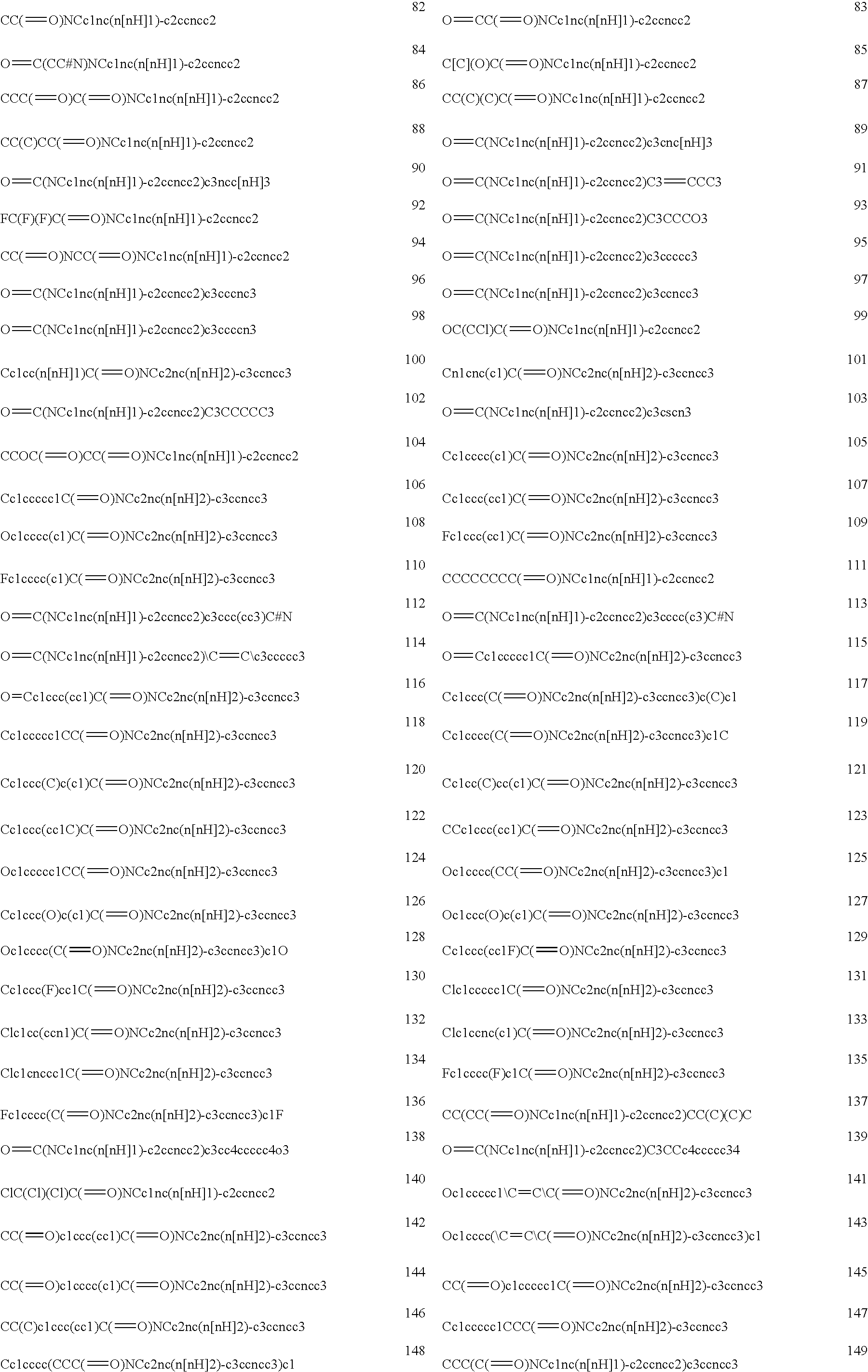

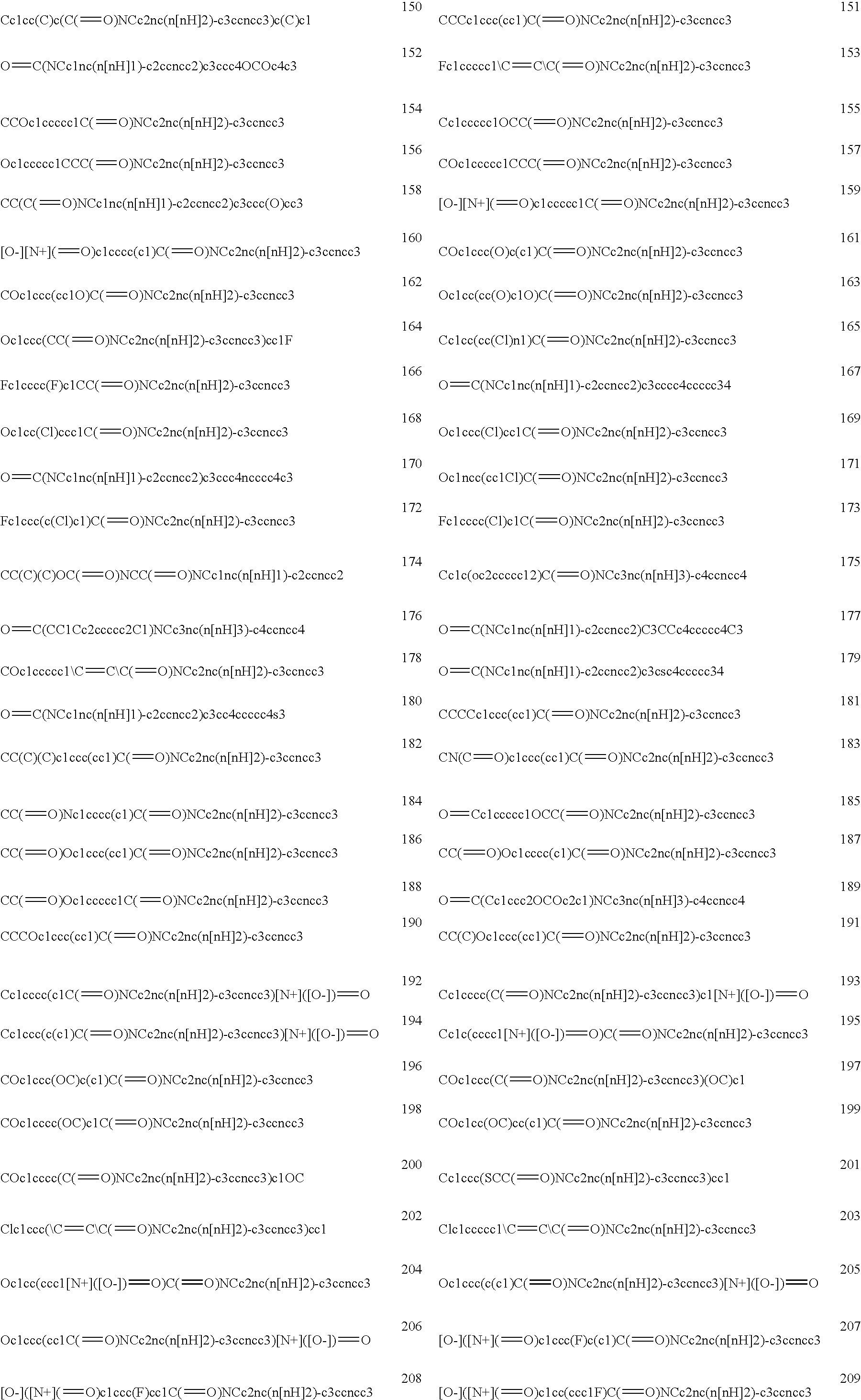

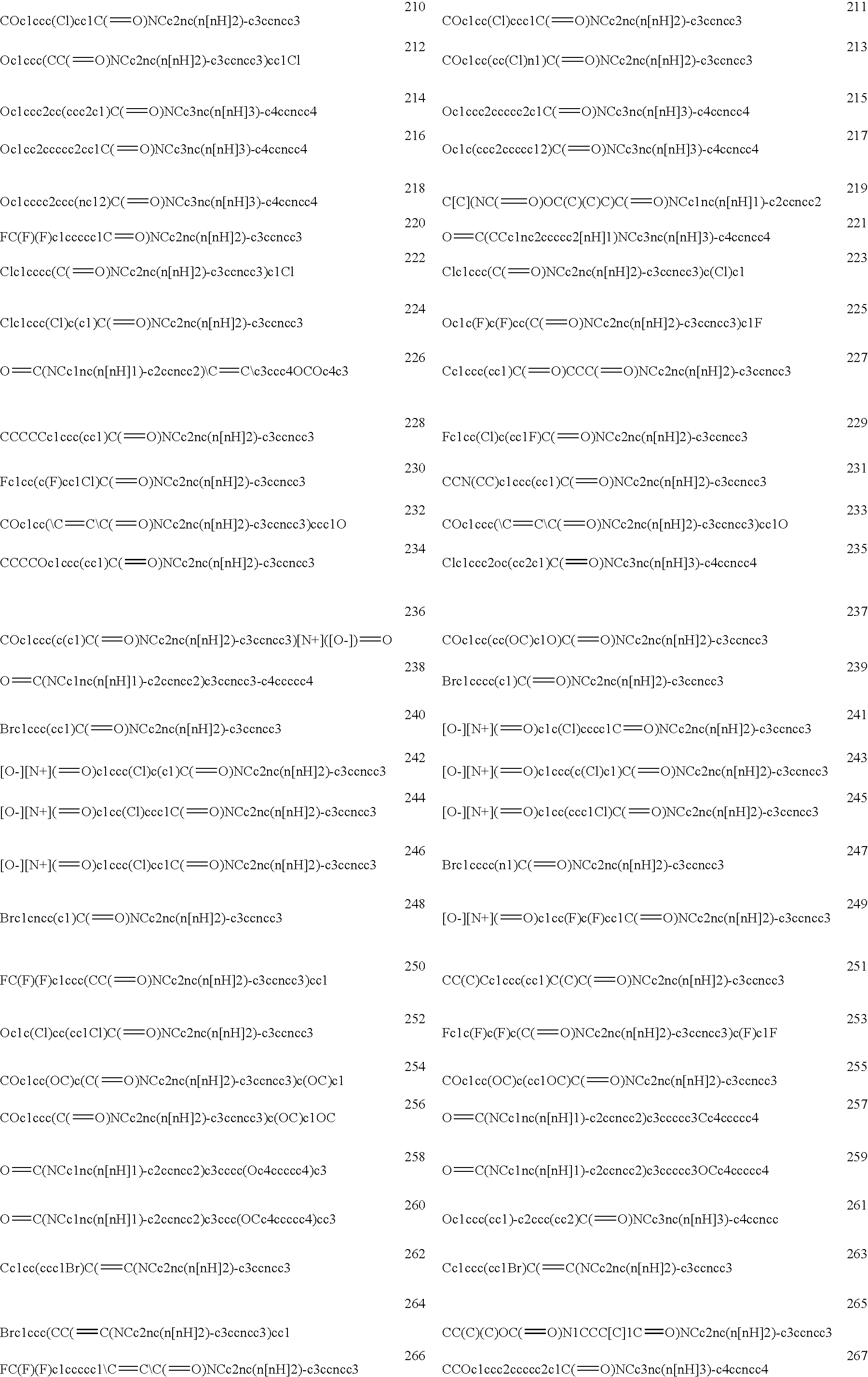

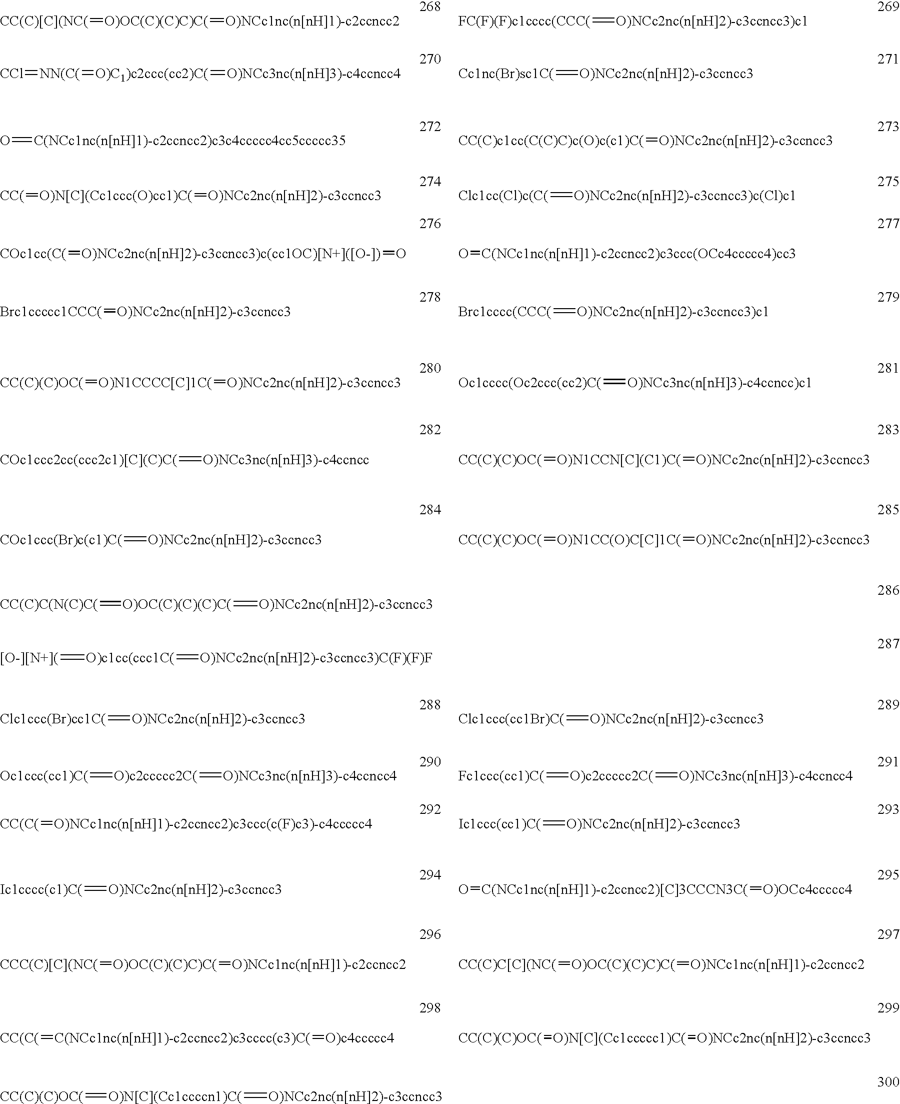

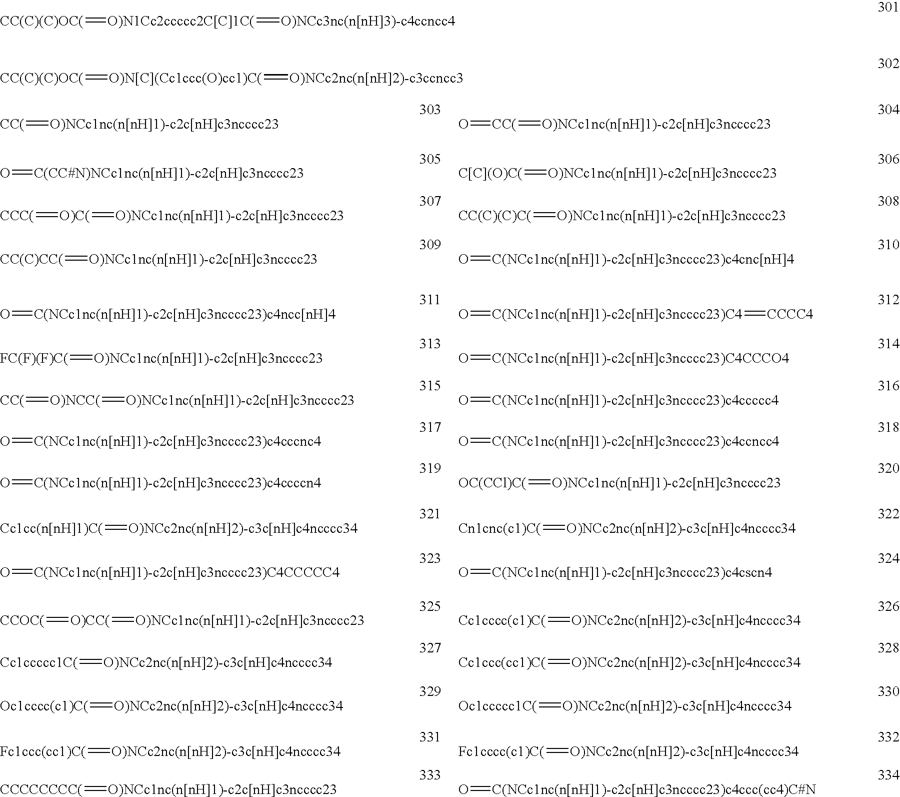

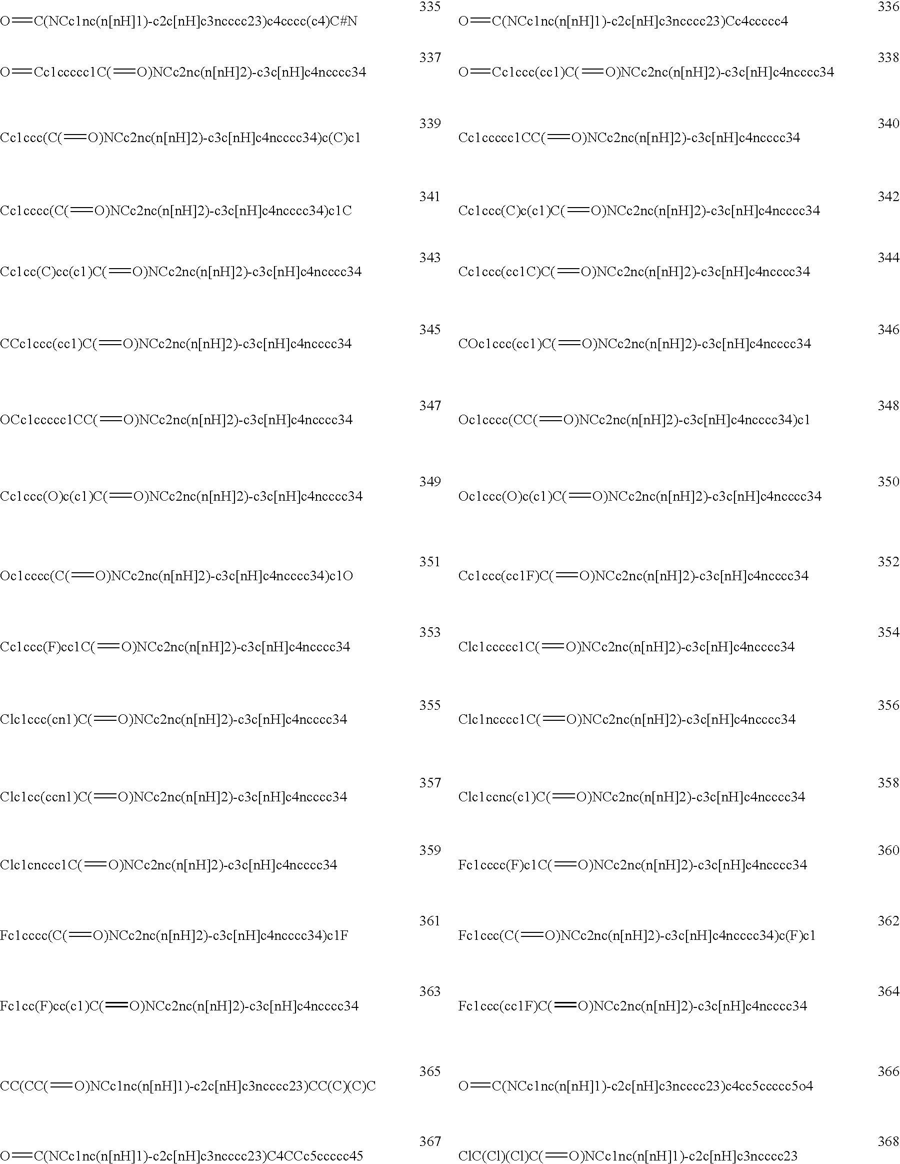

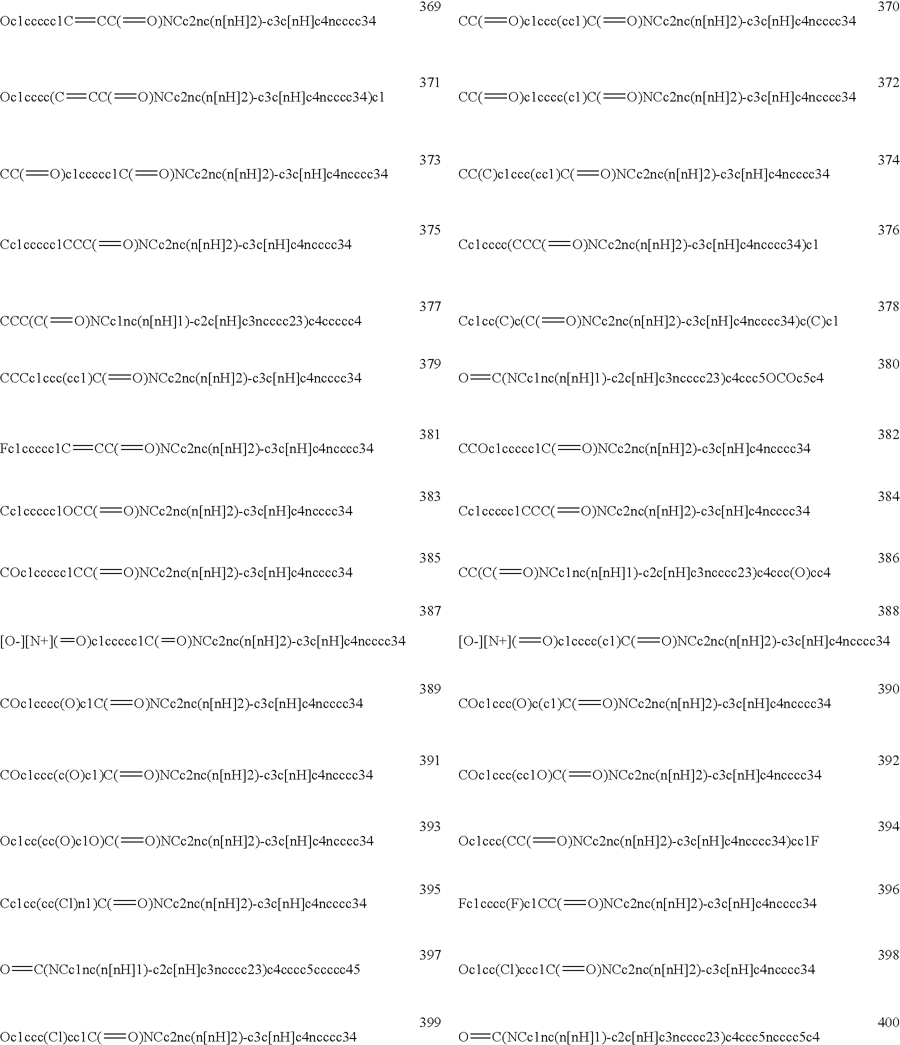

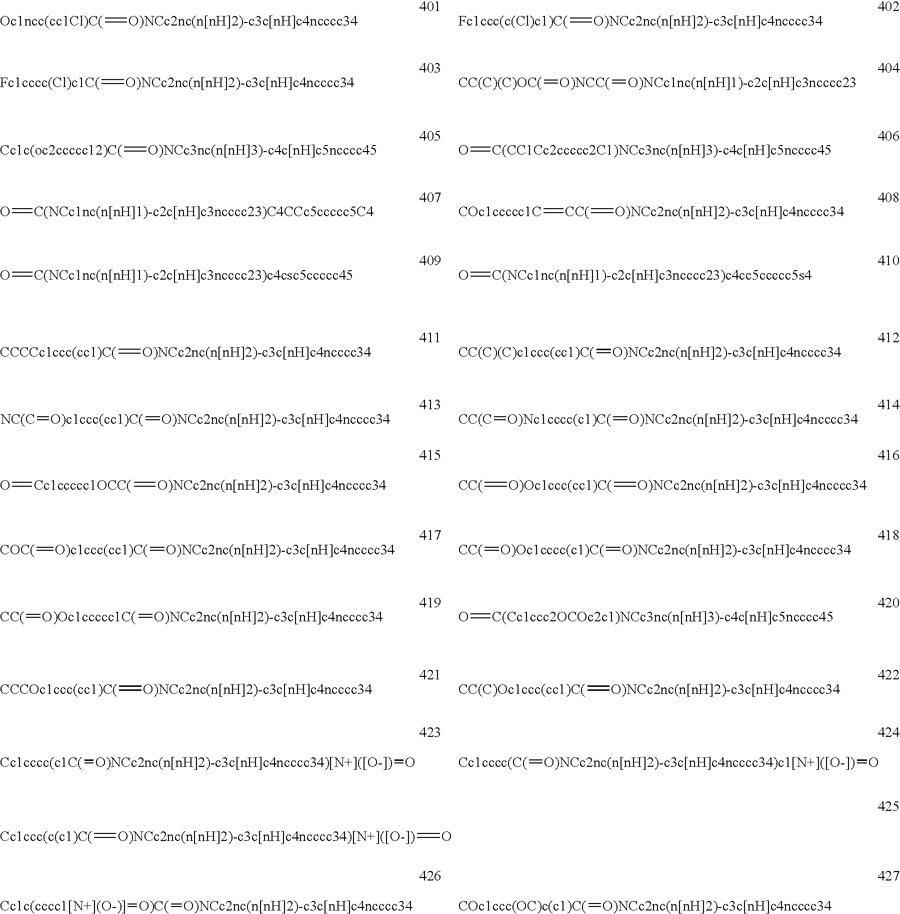

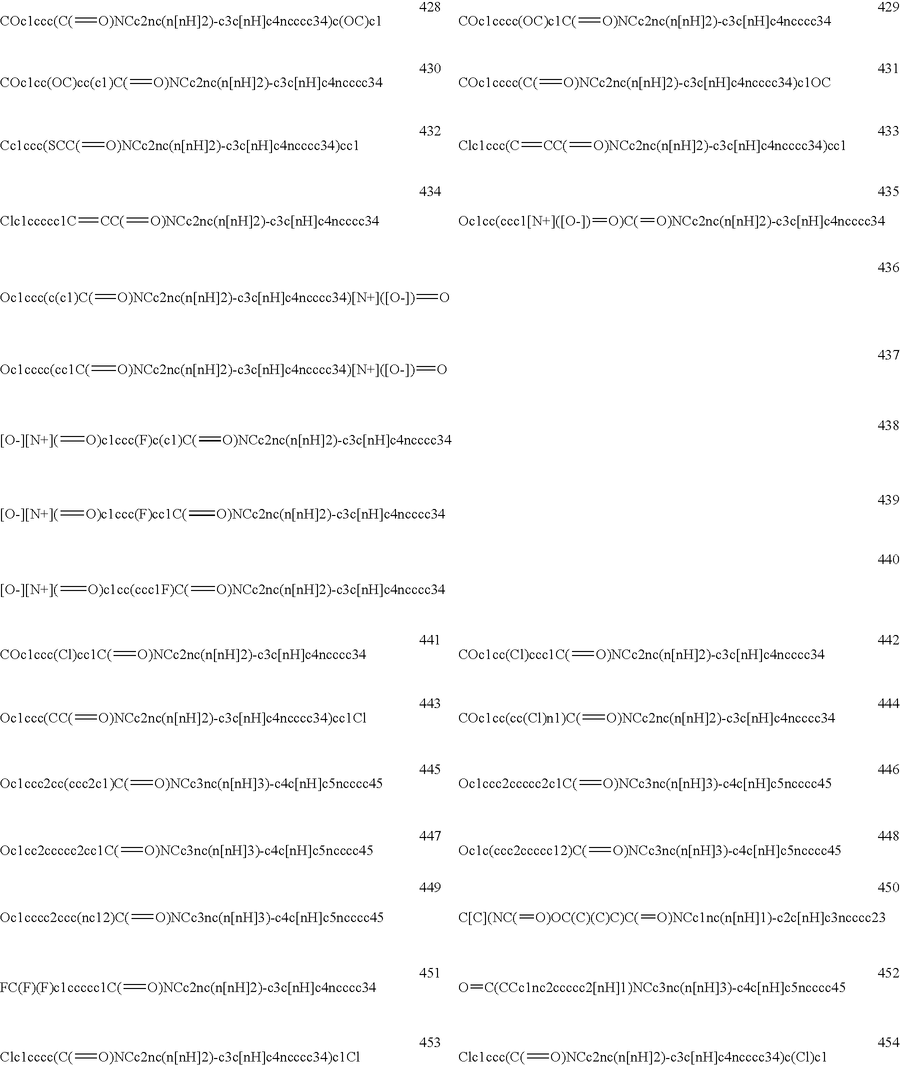

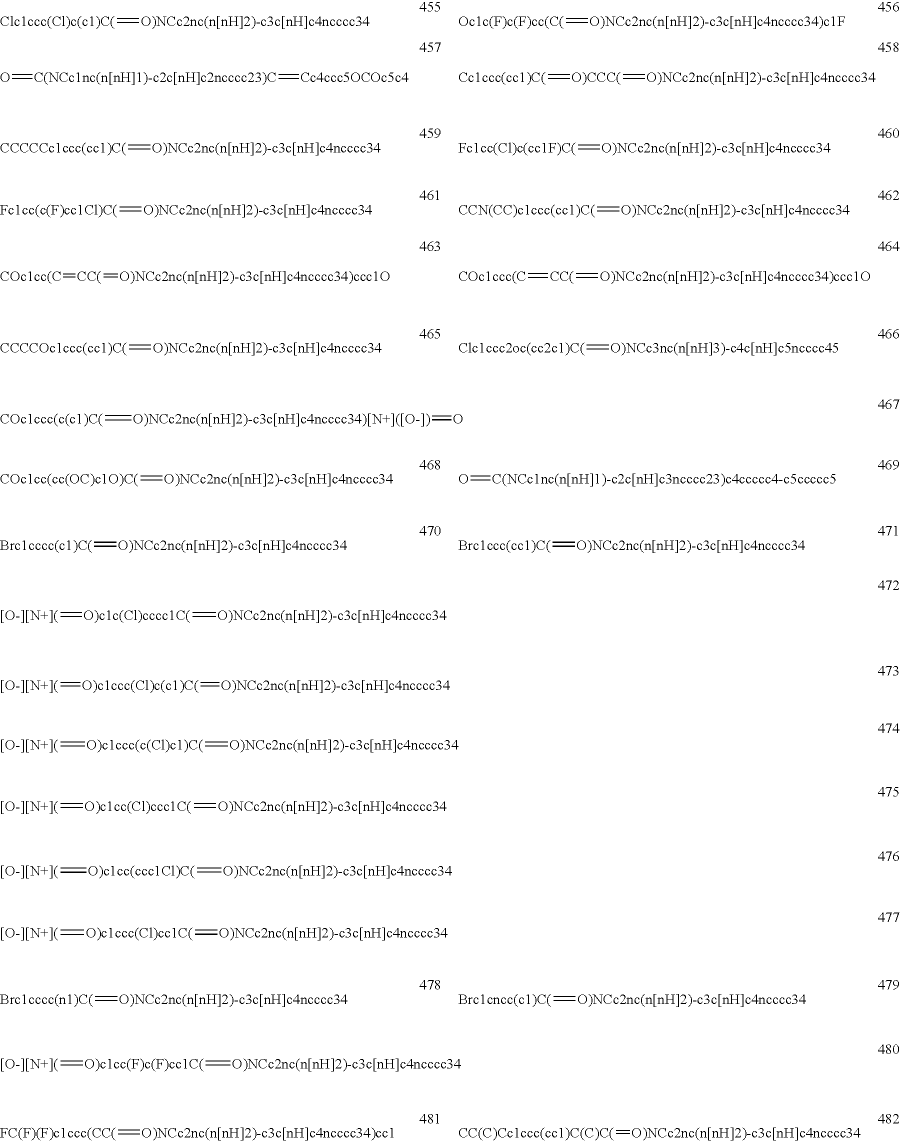

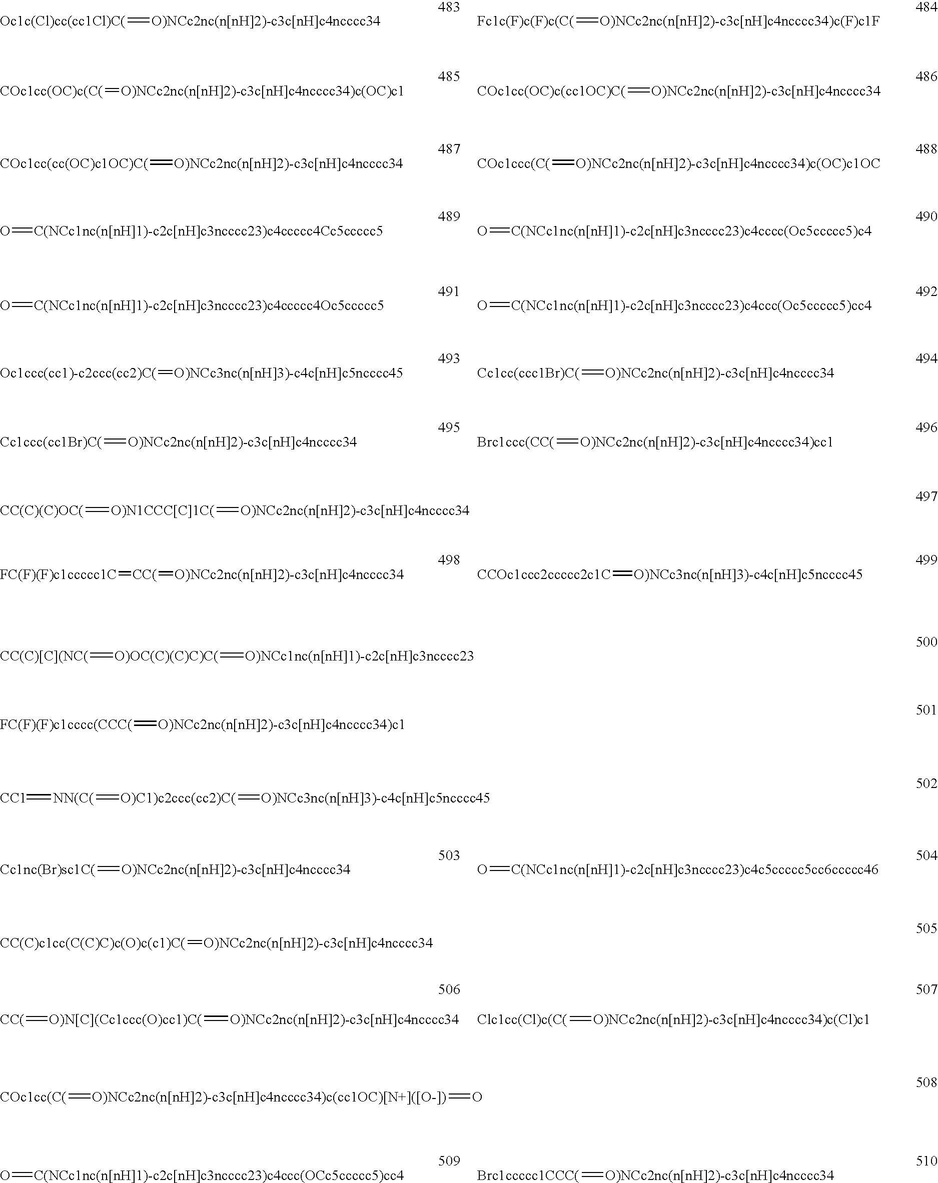

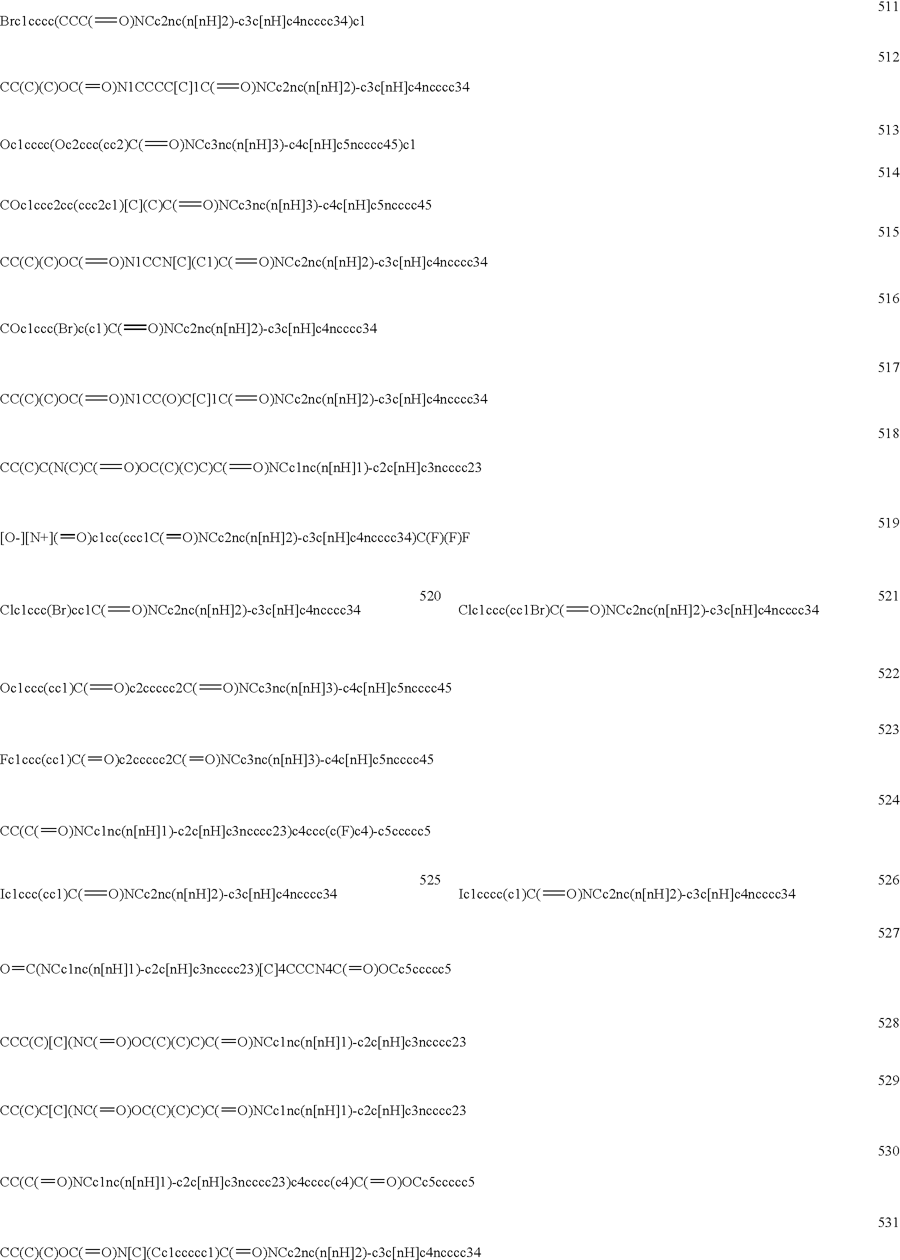

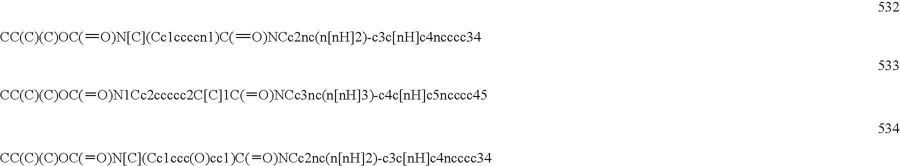

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as many IUPAC names, were generated using CambridgeSoft's ChemDraw 10.0.

The following compounds can generally be made using the methods described above. It is expected that these compounds when made will have activity similar to those that have been made in the examples above.
O═C(C1=C(O)C═C(O)C═C1)N[C@@H](C)C2=NC(C3=CC═NC═C3)=NN2
O═C(C4=C(O)C(C)═C(O)C═C4)N[C@@H](C)C5=NC(C6=CC═NC═C6)=NN5
O═C(C7=CC═C(O)C═C7)N[C@@H](C)C8=NC(C9=CC═NC═C9)=NN8
O═C(C %10=C(O)C═C(O)C═C %10)N[C@H](C)C %11=NC(C %12=CC═NC═C %12)=NN %11
O═C(C %13=C(O)C(C)═C(O)C═C %13)N[C@H](C)C %14=NC(C %15=CC═NC═C %15)=NN %14
O═C(C %16=CC═C(O)C═C %16)N[C@H](C)C %17=NC(C %18=CC═NC═C %18)=NN %17
O═C(C %19=C(O)C(C)═C(O)C═C %19)NCC %20=NC(C %21=CC═NC═C %21)=CN %20
O═C(C %22=CC═C(O)C═C %22)NCC %23=NC(C %24=CC═NC═C %24)=CN %23
O═C(C %25=C(O)C═C(O)C═C %25)N[C@@H](C)C %26=NC(C %27=CC═NC═C %27)=CN %26
O═C(C1=C(O)C(C)═C(O)C═C1)N[C@@H](C)C2=NC(C3=CC═NC═C3)=CN2
O═C(C4=CC═C(O)C═C4)N[C@@H](C)C5=NC(C6=CC═NC═C6)=CN5
O═C(C7=C(O)C═C(O)C═C7)N[C@H](C)C8=NC(C9=CC═NC═C9)=CN8
O═C(C %10=C(O)C(C)═C(O)C═C %10)N[C@H](C)C %11=NC(C %12=CC═NC═C %1 2)=CN %1
O═C(C %13=CC═C(O)C═C %13)N[C@H](C)C %14=NC(C %15=CC═NC═C %15)=CN %14
O═C(C %16=C(O)C(C)═C(O)C═C %16)NCC %17=CC(C %18=CC═NC═C %18)=NN %17
O═C(C %19=CC═C(O)C═C %19)NCC %20=CC(C %21=CC═NC═C %21)=NN %20
O═C(C %22=C(O)C═C(O)C═C %22)N[C@@H](C)C %23=CC(C %24=CC═NC═C %24)=NN %23
O═C(C %25=C(O)C(C)═C(O)C═C %25)N[C@@H](C)C %26=CC(C %27=CC═NC═C %27)=NN %26
O═C(C1=C(O)C(C)═C(O)C═C1)N[C@@H](C)C2=NC(C3=CC═NC═C3)=CN2
O═C(C4=CC═C(O)C═C4)N[C@@H](C)C5=NC(C6=CC═NC═C6)=CN5
O═C(C7=C(O)C═C(O)C═C7)N[C@H](C)C8=NC(C9=CC═NC═C9)=CN8
O═C(C %10=C(O)C(C)═C(O)C═C %10)N[C@H](C)C %11=NC(C %12=CC═NC═C %12)=CN %11
O═C(C %13=CC═C(O)C═C %13)N[C@H](C)C %14=NC(C %15=CC═NC═C %15)=CN %14
O═C(C %16=C(O)C(C)═C(O)C═C %16)NCC %17=CC(C %18=CC═NC═C %18)=NN %17
O═C(C %19=CC═C(O)C═C %19)NCC %20=CC(C %21=CC═NC═C %21)=NN %20
O═C(C %22=C(O)C═C(O)C═C %22)N[C@@H](C)C %23=CC(C %24=CC═NC═C %24)=NN %23
O═C(C %25=C(O)C(C)═C(O)C═C %25)N[C@@H](C)C %26=CC(C %27=CC═NC═C %27)=NN %26
O═C(C1=C(O)C═C(O)C═C1)N[C@H](C)C2=NC(C3=CNC4=C3C═CC═N4)=NN2
O═C(C5=C(O)C(C)═C(O)C═C5)N[C@H](C)C6=NC(C7=CNC8=C7C═CC═N8)=NN6
O═C(C9=CC═C(O)C═C9)N[C@H](C)C %10=NC(C %11=CNC %12=C %11C═CC═N %12)=NN %10
O═C(C %13=C(O)C═C(O)C═C %13)NCC %14=NC(C %15=CNC %16=C %15C═CC═N %16)=CN %14
O═C(C %17=C(O)C(C)═C(O)C═C %17)NCC %18=NC(C %19=CNC %20=C %19C═CC═N %20)=CN %18
O═C(C %21=CC═C(O)C═C %21)NCC %22=NC(C %23=CNC %24=C %23C═CC═N %24)=CN %22
O═C(C %25=C(O)C═C(O)C═C %25)N[C@@H](C)C %26=NC(C %27=CNC %28=C %27C═CC═N %28)=CN %26
O═C(C %29=C(O)C(C)═C(O)C═C %29)N[C@@H](C)C %30=NC(C %31=CNC %32=C %31C═CC═N %32)=CN %30
O═C(C %33=CC═C(O)C═C %33)N[C@@H](C)C %34=NC(C %35=CNC %36=C %35C═CC═N %36)=CN %34
O═C(C1=C(O)C═C(O)C═C1)N[C@H](C)C2=NC(C3=CNC4=C3C═CC═N4)=CN2
O═C(C5=C(O)C(C)═C(O)C═C5)N[C@@H](C)C6=NC(C7=CNC8=C7C═CC═N8)=CN6
O═C(C9=CC═C(O)C═C9)N[C@H](C)C %10=NC(C %11=CNC %12=C %11C═CC═N %12)=CN %10.O═C(C %13=C(O)C═C(O)C═C %13)NCC %14=CC(C %15=CNC %16=C %15C═CC═N %16)=NN %14
O═C(C %17=C(O)C(C)═C(O)C═C %17)NCC %18=CC(C %19=CNC %20=C %19C═CC═N %20)=NN %18
O═C(C %21=CC═C(O)C═C %21)NCC %22=CC(C %23=CNC %24=C %23C═CC═N %24)=NN %22
O═C(C %25=C(O)C═C(O)C═C %25)N[C@@H](C)C %26=CC(C %27=CNC %28=C %27C═CC═N %28)=NN %26
O═C(C %29=C(O)C(C)═C(O)C═C %29)N[C@@H](C)C %30=CC(C %31=CNC %32=C %31C═CC═N %32)=NN %30
O═C(C %33=CC═C(O)C═C %33)N[C@@H](C)C %34=CC(C %35=CNC %36=C %35C═CC═N %36)=NN %34
O═C(C1=C(O)C═C(O)C═C1)N[C@H](C)C2=CC(C3=CNC4=C3C═CC═N4)=NN2
O═C(C5=C(O)C(C)═C(O)C═C5)N[C@H](C)C6=CC(C7=CNC8=C7C═CC═N8)=NN6
O═C(C9=CC═C(O)C═C9)N[C@H](C)C %10=CC(C %11=CNC %12=C %11C═CC═N %12)=NN %10
O═C(C %13=C(O)C═C(O)C═C %13)NCC %14=NC(C %15=CC═NC %16=C %15C═CN %16)=NN %14
O═C(C %17=C(O)C═C(O)C═C %17)NCC %18=NC(C %19=CC(N)═NC═C %19)=NN %18
O═C(C %20=C(O)C═C(O)C═C %20)NCC %21=NC(C %22=CC(N)═NC(N)=C %22)=NN %21
O═C(C %23=C(O)C═C(O)C═C %23)NCC %24=NC(C %25=CC═NC %26=C %25N═CN %26)=NN %24
O═C(C %27=C(O)C═C(O)C═C %27)NCC %28=NC(C %29=NC(N)═NC═C %29)=NN %28
O═C(C %30=C(O)C═C(O)C═C %30)NCC %31=NC(C %32=NON=C %32N)═NN %31
O═C(C1=C(O)C═C(O)C═C1)NCC2=CC(C3=CC═NC═C3)=CN2
O═C(C4=C(O)C═C(O)C═C4)NCC5=NC(C6=CC═NC═C6)=NO5
O═C(C7=C(O)C(C)═C(O)C═C7)NCC8=NC(C9=CC═NC═C9)=CO8
O═C(C %10=C(O)C═C(O)C═C %10)NCC %11=NC(C %12=CC═NC═C %12)=CS %11
O═C(C %13=C(O)C═C(O)C═C %13)NCC %14=CC═C(C %15=CC═NC═C %15)N %14
O═C(C %16=C(O)C═C(O)C═C %16)NCC %17=NN=C(C %18=CC═NC═C %18)O %17
O═C(C %19=C(O)C═C(OC(CN %20CCOCC %20)=O)C═C %19)NCC %21=NC(C %22=CC═NC═C %22)=NN %21
O═C(C %23=C(O)C═C(OC(CCN %24CCN(C)CC %24)=O)C═C %23)NCC %25=NC(C %26=CC═NC═C %26)=NN %25
O═C(C %27=C(O)C═C(OC(CN(CC)CC)═O)C═C %27)NCC %28=NC(C %29=CC═NC═C %29)=NN %28
OC[C@H](NC(C1=CC═C(O)C═C1O)═O)C2=NC(C3=CC═NC═C3)=NN2
O═C(C4=CC═C(O)C═C40)CCC5=NC(C6=CC═NC═C6)=NN5
O═C(C7=CC═C(O)C═C70)N[C@@H](CC8=CC═C(O)C═C8)C9=NC(C %10=CC═NC═C %10)=NN9
O═C(C %11=CC═C(O)C═C %11O)N[C@H](CC %12=CNC %13=C %12C═CC═C %13)C %14=NC(C %15=CC═NC═C %15)=NN %14
O═C(C %16=CC═C(O)C═C %160)N[C@H](CCC(O)═O)C %17=NC( C%18=CC═NC═C %18)=NN %17
OC[C@@H](NC(C %19=CC═C(O)C═C %190)=O)C %20=NC(C %21=CC═NC═C %21)=NN %20
O═C(C %22=CC═C(O)C═C %220)N[C@H](CC %23=CC═C(O)C═C %23)C %24=NC(C %25=CC═NC═C %25)=NN %24
O═C(C %26=CC═C(O)C═C %260)N[C@@H](CC %27=CNC %28=C %27C═CC═C %28)C %29=NC(C %30=CC═NC═C %30)=NN %29
O═C(C %31=CC═C(O)C═C %310)N[C@@H](CCC(O)═O)C %32=NC(C %33=CC═NC═C %33)=NN %32
OC1=CC(O)═CC═C1C2=NC═C(C3=NC(C4=CC═NC═C4)=NN3)N2
O═C(C5=CC═C(O)C═C50)N[C@@H](CCCCN)C6=NC(C7=CC═NC═C7)=NN6
O═C(C8=CC═C(O)C═C80)N[C@@H](CC9=CNC═N9)C %10=NC(C %11=CC═NC═C %11)=NN %10
O═C(C %12=CC═C(O)C═C %120)N[C@H](CC(O)═O)C %13=NC(C %14=CC═NC═C %14)=NN %13
OC %15=CC(O)═CC═C %15C %16=NC(C %17=NC(C %18=CC═NC═C %18)=NN %17)=CO %16
O═C(C %19=CC═C(O)C═C %19O)N[C@H](CCCCN)C %20=NC(C %21=CC═NC═C %21)=NN %20
O═C(C %22=CC═C(O)C═C %220)N[C@H](CC %23=CNC═N %23)C %24=NC(C %25=CC═NC═C %25)=NN %24
O═C(C %26=CC═C(O)C═C %260)N[C@@H](CC(O)═O)C %27=NC(C %28=CC═NC═C %28)=NN %27
O═C(C %29=C(O)C═C(O)C═C %29)N[C@@H](CN)C %30=NC(C %31=CC═NC═C %31)=NN %30
O═C(C1=C(O)C═C(O)C═C1)N[C@@H](CNC)C2=NC(C3=CC═NC═C3)=NN2
O═C(C4=C(O)C═C(O)C═C4)N[C@@H](CN(C)C)C5=NC(C6=CC═NC═C6)=NN5
O═C(C7=C(O)C═C(O)C═C7)N[C@@H](CN8CCOCC8)C9=NC(C %10=CC═NC═C %10)=NN9
O═C(C %11=C(O)C═C(O)C═C %11)N[C@@H](CCN)C %12=NC(C %13=CC═NC═C %13)=NN %12
O═C(C %14=C(O)C═C(O)C═C %14)N[C@H](CN)C %15=NC(C %16=CC═NC═C %16)=NN %15
O═C(C %17=C(O)C═C(O)C═C %17)N[C@H](CNC)C %18=NC(C %19=CC═NC═C %19)=NN %18
O═C(C %20=C(O)C═C(O)C═C %20)N[C@H](CN(C)C)C %21=NC(C %22=CC═NC═C %22)=NN %21
O═C(C %23=C(O)C═C(O)C═C %23)N[C@H](CN %24CCOCC %24)C %25=NC(C %26=CC═NC═C %26)=NN %25
O═C(C %27=C(O)C═C(O)C═C %27)N[C@H](CCN)C %28=NC(C %29=CC═NC═C %29)=NN %28
O═C(C1=C(O)C═C(O)C═C1)N[C@@H](CCN(C)C)C2=NC(C3=CC═NC═C3)=NN2
O═C(C4=C(O)C═C(O)C═C4)N[C@@H](CCN5CCOCC5)C6=NC(C7=CC═NC═C7)=NN6
O═C(C8=C(O)C═C(O)C═C8)N[C@@H](CCCN)C9=NC(C %10=CC═NC═C %10)=NN9
O═C(C %11=C(O)C═C(O)C═C %11)N[C@@H](CCCNC)C %12=NC(C %13=CC═NC═C %13)=NN %12
O═C(C %14=C(O)C═C(O)C═C %14)N[C@H](CCN(C)C)C %15=NC(C %16=CC═NC═C %16)=NN %15
O═C(C %17=C(O)C═C(O)C═C %17)N[C@H](CCN %18CCOCC %18)C %19=NC(C %20=CC═NC═C %20)=NN %19
O═C(C %21=C(O)C═C(O)C═C %21)N[C@H](CCCN)C %22=NC(C %23=CC═NC═C %23)=NN %22
O═C(C %24=C(O)C═C(O)C═C %24)N[C@@H](CCNC)C %25=NC(C %26=CC═NC═C %26)=NN %25
O═C(C %27=C(O)C═C(O)C═C %27)N[C@H](CCNC)C %28=NC(C %29=CC═NC═C %29)=NN %28
O═C(C1=C(O)C═C(O)C═C1)N[C@@H](CCCN(C)C)C2=NC(C3=CC═NC═C3)=NN2
O═C(C4=C(O)C═C(O)C═C4)N[C@@H](CCCN5CCOCC5)C6=NC(C7=CC═NC═C7)=NN6
O═C(C8=C(O)C═C(O)C═C8)N[C@H](CCCNC)C9=NC(C %10=CC═NC═C %10)=NN9
O═C(C %11=C(O)C═C(O)C═C %11)N[C@H](CCCN(C)C)C %12=NC(C %13=CC═NC═C %13)=NN %12
O═C(C %14=C(O)C═C(O)C═C %14)N[C@H](CCCN %15CCOCC %15)C %16=NC(C %17=CC═NC═C %17)=NN %16
O═C(C %18=CC═C(CO)C═C %18)NCC %19=NC(C %20=CC═NC═C %20)=CN %19
O═C(C %21=CC═C(C(C)(C)O)C═C %21)NCC %22=NC(C %23=CC═NC═C %23)=NN %22
O═C(C %24=CC(CO)═CC═C %24)NCC %25=NC(C %26=CC═NC═C %26)=NN %25
O═C(C %27=CC(C(C)(C)O)═CC═C %27)NCC %28=NC(C %29=CC═NC═C %29)=NN %28
OC(C═C1)=CC2=C1C(NCC3=NC(C4=CC═NC═C4)=NN3)=NO2
O═C(C5=C(CO)C═CC═C5)NCC6=NC(C7=CC═NC═C7)=NN6
O═C(C8=CC═CS8)NCC9=CC(C %10=CC═NC═C %10)=NN9
O═C(C %11=CC═CN %11)NCC %12=NC(C %13=CC═NC═C %13)=NN %12
O═C(C %14=CN═CN %14)NCC %15=NC(C %16=CC═NC═C %16)=CN %15
O═C(C %17=C(O)C═CC═C %17O)NCC %18=NC(C %19=CC═NC═C %19)=NN %18
O═C(C %20=C(O)C═C(OC)C═C %20)NCC %21=NC(C %22=CC═NC═C %22)=CN %21
O═C(C %23=CC═C(OC)C═C %23)NCC %24=NC(C %25=CC═NC═C %25)=CN %24
O═C(C %26=C(O)C═C(OC(C)═O)C═C %26)NCC %27=NC(C %28=CC═NC═C %28)=CN %27
O═C(C1=C(O)C═C(C)C═C1)NCC2=NC(C3=CC═NC═C3)=CN2
O═C(C4=CC═C(C)S4)NCC5=NC(C6=CC═NC═C6)=CN5
O═C(C7=C(C)C═CS7)NCC8=NC(C9=CC═NC═C9)=CN8
O═C(C %10=CC═C(C(OC)═O)C═C %10)NCC %11=NC(C %12=CC═NC═C %12)=CN %11
O═C(C %13=CC(C)═CS %13)NCC %14=NC(C %15=CC═NC═C %15)=CN %14
O═C(C %16=CC═C(CO)S %16)NCC %17=NC(C %18=CC═NC═C %18)=NN %17
O═C(C %19=CC(CO)═CS %19)NCC %20=NC(C %21=CC═NC═C %21)=NN %20
O═C(C %22=C(CO)C═CS %22)NCC %23=NC(C %24=CC═NC═C %24)=NN %23
O═C(C %25=C(O)C(C)═CC═C %25)NCC %26=NC(C %27=CNC %28=C %27C═CC═N %28)=CN %26
O═C(C1=C(O)C═C(OC([C@H](CO)N)═O)C═C1)NCC2=NC(C3=CC═NC═C3)=NN2
O═C(C4=C(O)C═C(OC([C@H]5N(C)CCC5)=O)C═C4)NCC6=NC(C7=CC═NC═C7)=NN6
O═C(C8=C(O)C═C(OC([C@@H](CO)N)═O)C═C8)NCC9=NC(C %10=CC═NC═C %10)=NN9
O═C(C %11=C(O)C═C(OC([C@@H]%12N(C)CCC %12)=O)C═C %11)NCC %13=NC(C %14=CC═NC═C %14)=NN %13
O═C(C %15=C(O)C═C(C(OC)═O)C═C %15)NCC %16=NC(C %17=CC═NC═C %17)=NN %16

The activity of the compounds in Examples 1-534 as Rho kinase inhibitor is illustrated in the following assay. The other compounds listed above, which have not yet been made or tested, are predicted to have activity in this assay as well.

### Biological Activity Assay

In Vitro Rho Kinase Assay

Rho kinase biochemical assays described below depend on firefly luciferase-based, indirect measurement of total ATP consumption by the kinase following incubation with substrate and ATP. 25 μl of Rho kinase assay buffer (20 mM Tris-HCL [pH 7.5], 1 mM MgCl₂, 0.4 mM CaCl₂, 0.15 mM EGTA, 0.1 mg/ml bovine serum albumin) containing 0.82 μg/ml of recombinant N-terminal GST-tagged human Rho kinase 1 (ROCK1, amino acids 1-535, Invitrogen Inc., cat. #PV-3691) or recombinant N-terminal GST-tagged human Rho kinase 2 (ROCK2, amino acids 1-552, Invitrogen Inc., cat #PV3759), 100 μg/ml S6 peptide substrate (related to amino acids 218-249 of the human 40S ribosomal protein S6, and suitable for ROCK1 or ROCK2, e.g. Upstate/Millipore Inc., cat #12-420), and 3 M ATP are dispensed to wells of a 384 multi-well opaque plate. The plate is centrifuged for 30 seconds at approximately 200×g. 240 nl of test compound in DMSO is dispensed to each well by passive pin transfer. The lag phase of this in vitro kinase reaction permits addition of compounds soon after the reaction initiates. The reaction is allowed to incubate at 30° C. for 2 hours. The assay plates are sealed and maintained in a humidified environment. After 2 hours, 25 μl of easyLite protein kinase assay reagent (Perkin-Elmer, Inc.) is dispensed. After an additional 10 minute incubation at room temperature (about 22° C.), luminescence activity is measured on a Molecular Devices Analyst multi-mode plate reader or other suitable plate reader. Kinase inhibition results in less ATP consumption, and therefore increased luminescence signal. Negative control activity is measured with DMSO lacking any test compound. The positive control is 2-methyl-1-(4-methylisoquinolin-5-ylsulfonyl)perhydro-1,4-diazepine hydrochloride (aka H-1152P, HCl salt). Efficacy is measured as a percentage of positive control activity. 50% inhibitory concentration of compound (IC50) is measured by assay in dose response. In some cases, kinase reactions and compound testing are performed in 1536 multi-well plates under similar conditions, with assay volumes appropriately scaled. The designated NT means the cited example was not tested.

TABLE 1
Biological Activity

| | ROCK1 IC₅₀ | ROCK2 IC₅₀ |
|---|---|---|
| | + indicates | + indicates |
| | ≦5 uM | ≦5 uM |
| | − indicates | − indicates |
| Example | >5 uM | >5 uM |
| 1 | − | − |
| 2 | − | − |
| 3 | − | − |
| 4 | − | − |
| 5 | + | + |
| 6 | + | + |
| 7 | + | + |
| 8 | + | + |
| 9 | + | + |
| 10 | + | + |
| 11 | − | − |
| 12 | + | + |
| 13 | − | − |
| 14 | − | − |
| 15 | + | + |
| 16 | + | + |
| 17 | + | + |
| 18 | − | − |
| 19 | + | + |
| 20 | + | + |
| 21 | − | − |
| 22 | + | + |
| 23 | + | + |
| 24 | + | + |
| 25 | + | + |
| 26 | + | + |
| 27 | + | + |
| 28 | + | + |
| 29 | − | − |
| 30 | + | + |
| 31 | − | + |
| 32 | − | − |
| 33 | − | + |
| 34 | NA | − |
| 35 | + | + |
| 36 | + | + |
| 37 | − | − |
| 38 | − | − |
| 39 | + | − |
| 40 | − | − |
| 41 | + | + |
| 42 | + | + |
| 43 | + | − |
| 44 | − | − |
| 45 | + | + |
| 46 | + | + |
| 47 | + | + |
| 48 | + | + |
| 49 | + | + |
| 50 | − | − |
| 51 | + | + |
| 52 | + | + |
| 53 | − | − |
| 54 | − | − |
| 55 | − | − |
| 56 | − | − |
| 57 | − | − |
| 58 | − | − |
| 59 | − | − |
| 60 | NA | − |
| 61 | NA | − |
| 62 | − | − |
| 63 | + | + |
| 64 | − | − |
| 65 | + | + |
| 66 | + | + |
| 67 | − | − |
| 68 | NA | − |
| 69 | + | + |
| 70 | + | + |
| 71 | + | − |
| 72 | + | + |
| 73 | + | + |
| 74 | + | + |
| 75 | + | + |
| 76 | + | + |
| 77 | + | + |
| 78 | + | + |
| 79 | + | + |
| 80 | + | + |
| 81 | NA | NA |
| 82 | NA | NA |
| 83 | NA | NA |
| 84 | − | NA |
| 85 | − | NA |
| 86 | − | − |
| 87 | NA | NA |
| 88 | NA | NA |
| 89 | − | − |
| 90 | − | − |
| 91 | − | − |
| 92 | − | − |
| 93 | − | − |
| 94 | − | − |
| 95 | − | − |
| 96 | − | − |
| 97 | − | − |
| 98 | NA | − |
| 99 | NA | − |
| 100 | NA | NA |
| 101 | NA | NA |
| 102 | NA | − |
| 103 | − | − |
| 104 | NA | NA |
| 105 | − | − |
| 106 | − | − |
| 107 | − | − |
| 108 | − | − |
| 109 | − | − |
| 110 | − | − |
| 111 | − | − |
| 112 | − | − |
| 113 | − | − |
| 114 | − | − |
| 115 | − | − |
| 116 | − | − |
| 117 | NA | NA |
| 118 | NA | NA |
| 119 | NA | − |
| 120 | NA | − |
| 121 | NA | − |
| 122 | NA | NA |
| 123 | NA | NA |
| 124 | − | − |
| 125 | − | − |
| 126 | + | − |
| 127 | − | + |
| 128 | − | − |
| 129 | − | − |
| 130 | − | − |
| 131 | − | − |
| 132 | − | − |
| 133 | − | − |
| 134 | − | − |
| 135 | − | − |
| 136 | − | − |
| 137 | − | − |
| 138 | − | − |
| 139 | − | − |
| 140 | − | − |
| 141 | − | − |
| 142 | + | + |
| 143 | − | − |
| 144 | − | − |
| 145 | − | − |
| 146 | − | − |
| 147 | − | − |
| 148 | − | − |
| 149 | − | − |
| 150 | − | − |
| 151 | − | − |
| 152 | − | − |
| 153 | − | − |
| 154 | − | − |
| 155 | − | − |
| 156 | − | − |
| 157 | − | − |
| 158 | − | − |
| 159 | − | − |
| 160 | − | − |
| 161 | + | + |
| 162 | − | − |
| 163 | − | − |
| 164 | − | − |
| 165 | − | − |
| 166 | − | − |
| 167 | NA | − |
| 168 | − | − |
| 169 | − | − |
| 170 | NA | − |
| 171 | − | − |
| 172 | − | − |
| 173 | − | − |
| 174 | − | − |
| 175 | − | − |
| 176 | − | − |
| 177 | − | − |
| 178 | − | − |
| 179 | − | − |
| 180 | + | + |
| 181 | − | − |
| 182 | − | − |
| 183 | − | − |
| 184 | − | − |
| 185 | − | − |
| 186 | + | + |
| 187 | − | − |
| 188 | + | + |
| 189 | − | − |
| 190 | − | − |
| 191 | − | − |
| 192 | − | − |
| 193 | − | − |
| 194 | − | − |
| 195 | − | − |
| 196 | − | − |
| 197 | − | − |
| 198 | − | − |
| 199 | − | − |
| 200 | − | − |
| 201 | − | − |
| 202 | − | − |
| 203 | − | − |
| 204 | + | + |
| 205 | − | − |
| 206 | − | − |
| 207 | − | − |
| 208 | − | − |
| 209 | − | − |
| 210 | − | − |
| 211 | − | − |
| 212 | − | − |
| 213 | − | − |
| 214 | − | + |
| 215 | − | − |
| 216 | + | + |
| 217 | + | + |
| 218 | − | − |
| 219 | − | − |
| 220 | − | − |
| 221 | − | − |
| 222 | − | − |
| 223 | − | − |
| 224 | NA | − |
| 225 | − | − |
| 226 | − | − |
| 227 | − | − |
| 228 | − | − |
| 229 | − | − |
| 230 | − | − |
| 231 | − | − |
| 232 | − | − |
| 233 | − | − |
| 234 | − | − |
| 235 | − | − |
| 236 | − | − |
| 237 | − | − |
| 238 | NA | − |
| 239 | − | − |
| 240 | − | − |
| 241 | + | + |
| 242 | − | − |
| 243 | − | − |
| 244 | − | − |
| 245 | − | − |
| 246 | − | − |
| 247 | NA | − |
| 248 | − | − |
| 249 | − | − |
| 250 | − | − |
| 251 | − | − |
| 252 | − | − |
| 253 | − | − |
| 254 | − | − |
| 255 | − | − |
| 256 | − | − |
| 257 | − | − |
| 258 | − | − |
| 259 | − | − |
| 260 | + | + |
| 261 | − | − |
| 262 | − | − |
| 263 | − | − |
| 264 | − | − |
| 265 | − | − |
| 266 | − | − |
| 267 | − | − |
| 268 | − | − |
| 269 | − | + |
| 270 | − | − |
| 271 | − | − |
| 272 | NA | − |
| 273 | NA | − |
| 274 | NA | − |
| 275 | NA | − |
| 276 | − | − |
| 277 | − | − |
| 278 | − | − |
| 279 | − | − |
| 280 | − | − |
| 281 | + | + |
| 282 | − | − |
| 283 | NA | − |
| 284 | NA | − |
| 285 | NA | − |
| 286 | NA | − |
| 287 | NA | − |
| 288 | NA | − |
| 289 | − | − |
| 290 | − | − |
| 291 | − | − |
| 292 | − | − |
| 293 | − | − |
| 294 | − | − |
| 295 | NA | − |
| 296 | − | − |
| 297 | NA | NA |
| 298 | − | − |
| 299 | − | − |
| 300 | − | − |
| 301 | − | − |
| 302 | − | − |
| 303 | − | − |
| 304 | − | − |
| 305 | − | − |
| 306 | − | − |
| 307 | − | − |
| 308 | − | − |
| 309 | − | − |
| 310 | − | − |
| 311 | − | − |
| 312 | − | − |
| 313 | − | − |
| 314 | − | − |
| 315 | − | − |
| 316 | − | − |
| 317 | − | − |
| 318 | − | − |
| 319 | − | − |
| 320 | − | − |
| 321 | − | − |
| 322 | − | − |
| 323 | − | − |
| 324 | − | − |
| 325 | − | − |
| 326 | − | − |
| 327 | − | − |
| 328 | − | − |
| 329 | − | − |
| 330 | + | − |
| 331 | − | − |
| 332 | − | − |
| 333 | − | − |
| 334 | − | − |
| 335 | − | − |
| 336 | − | − |
| 337 | − | − |
| 338 | − | − |
| 339 | − | − |
| 340 | − | − |
| 341 | − | − |
| 342 | − | − |
| 343 | − | − |
| 344 | − | − |
| 345 | + | − |
| 346 | − | − |
| 347 | − | − |
| 348 | − | − |
| 349 | − | − |
| 350 | − | − |
| 351 | − | − |
| 352 | − | − |
| 353 | − | − |
| 354 | − | − |
| 355 | − | − |
| 356 | − | − |
| 357 | − | − |
| 358 | − | − |
| 359 | − | − |
| 360 | − | − |
| 361 | − | − |
| 362 | − | − |
| 363 | − | − |
| 364 | − | − |
| 365 | − | − |
| 366 | − | − |
| 367 | − | − |
| 368 | − | − |
| 369 | − | − |
| 370 | − | + |
| 371 | − | − |
| 372 | − | − |
| 373 | − | − |
| 374 | − | − |
| 375 | − | − |
| 376 | − | − |
| 377 | − | − |
| 378 | − | − |
| 379 | − | − |
| 380 | − | − |
| 381 | − | − |
| 382 | − | − |
| 383 | − | − |
| 384 | − | − |
| 385 | − | − |
| 386 | − | − |
| 387 | − | − |
| 388 | − | − |
| 389 | − | − |
| 390 | + | − |
| 391 | + | − |
| 392 | − | − |
| 393 | − | − |
| 394 | − | − |
| 395 | − | − |
| 396 | − | − |
| 397 | − | − |
| 398 | − | − |
| 399 | − | − |
| 400 | − | − |
| 401 | − | − |
| 402 | − | − |
| 403 | − | − |
| 404 | − | − |
| 405 | − | − |
| 406 | − | − |
| 407 | − | − |
| 408 | − | − |
| 409 | − | − |
| 410 | − | − |
| 411 | − | − |
| 412 | − | − |
| 413 | − | − |
| 414 | − | − |
| 415 | − | − |
| 416 | + | + |
| 417 | − | − |
| 418 | − | − |
| 419 | + | − |
| 420 | − | − |
| 421 | − | − |
| 422 | − | − |
| 423 | − | − |
| 424 | − | − |
| 425 | − | − |
| 426 | − | − |
| 427 | − | − |
| 428 | − | − |
| 429 | − | − |
| 430 | − | − |
| 431 | − | − |
| 432 | − | − |
| 433 | − | − |
| 434 | − | − |
| 435 | − | − |
| 436 | − | − |
| 437 | − | − |
| 438 | − | − |
| 439 | − | − |
| 440 | − | − |
| 441 | − | − |
| 442 | − | − |
| 443 | − | − |
| 444 | − | − |
| 445 | − | − |
| 446 | − | − |
| 447 | − | − |
| 448 | − | − |
| 449 | − | − |
| 450 | − | − |
| 451 | − | − |
| 452 | − | − |
| 453 | − | − |
| 454 | − | − |
| 455 | − | − |
| 456 | − | − |
| 457 | − | − |
| 458 | − | − |
| 459 | − | − |
| 460 | − | − |
| 461 | + | − |
| 462 | + | − |
| 463 | + | − |
| 464 | − | − |
| 465 | + | − |
| 466 | − | − |
| 467 | − | − |
| 468 | + | − |
| 469 | + | − |
| 470 | + | − |
| 471 | + | − |
| 472 | + | − |
| 473 | + | − |
| 474 | − | − |
| 475 | − | − |
| 476 | + | − |
| 477 | + | − |
| 478 | + | − |
| 479 | − | − |
| 480 | − | − |
| 481 | − | − |
| 482 | − | − |
| 483 | − | − |
| 484 | − | − |
| 485 | − | − |
| 486 | − | − |
| 487 | − | − |
| 488 | − | − |
| 489 | − | − |
| 490 | − | − |
| 491 | − | − |
| 492 | − | − |
| 493 | − | − |
| 494 | − | − |
| 495 | − | − |
| 496 | − | − |
| 497 | NA | − |
| 498 | NA | − |
| 499 | − | − |
| 500 | − | − |
| 501 | − | NA |
| 502 | − | − |
| 503 | − | − |
| 504 | − | − |
| 505 | + | − |
| 506 | + | − |
| 507 | + | − |
| 508 | + | − |
| 509 | + | − |
| 510 | + | − |
| 511 | + | − |
| 512 | + | − |
| 513 | − | − |
| 514 | − | − |
| 515 | − | − |
| 516 | − | − |
| 517 | − | − |
| 518 | − | − |
| 519 | − | − |
| 520 | − | − |
| 521 | − | − |
| 522 | − | − |
| 523 | + | − |
| 524 | − | − |
| 525 | − | − |
| 526 | − | − |
| 527 | − | − |
| 528 | − | − |
| 529 | − | − |
| 530 | − | − |
| 531 | − | − |
| 532 | − | − |
| 533 | − | − |
| 534 | − | − |

### In Vivo Assay

Acute IOP Response in Lasered (Hypertensive) Eyes of Conscious Cynomolgus Monkeys

Intraocular pressure (IOP) can be determined with an Alcon Pneumatonometer after light corneal anesthesia with 0.1% proparacaine. Eyes are washed with saline after each measurement. After a baseline IOP measurement, test compound is instilled in one 30 pL aliquot to the right eyes only of nine cynomolgus monkeys. Vehicle is instilled in the right eyes of six additional animals. Subsequent IOP measurements are taken at 1, 3, and 6 hours, and peak reduction in IOP is reported below in Table 2 as percent of IOP lowering versus the control for each of the given concentrations of compound. NT indicates that the compound was not tested at a given concentration.

TABLE 2

Peak %
Reduction in
IOP vs. Control

| Example No. | at 0.3% | at 1.0% |
|---|---|---|
| 6 | 5.8 | NT |
| 7 | 11.0 | NT |
| 15 | 14.9 | 9.8 |
| Ex. 15 HCl | NT | 8.8 |
| 22 | 11.1 | NT |
| 35 | 8.6 | NT |
| 36 | 8.4 | NT |

A more detailed description of the assay used herein may be found in May et al., “Evaluation of the Ocular Hypotensive Response of Serotonin 5-HT_{1A} and 5-HT₂ Receptor Ligands in Conscious Ocular Hypertensive Cynomolgus Monkeys,” J. of Pharmacology and Experimental Therapeutics, vol. 306(1), pp. 301-309 (2003), the disclosure of which is hereby incorporated by reference as if written herein in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of inhibition of Rho kinase comprising contacting Rho kinase with a compound of structural Formula I 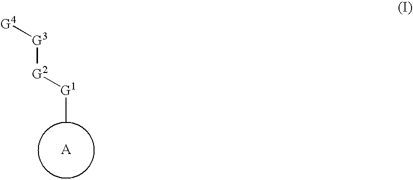
or a salt, ester, or prodrug thereof, wherein:
A is optionally substituted heteroaryl;
G¹ is optionally substituted 5-membered heteroaryl;
G2 is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted.

2. A method of inhibition of Rho kinase comprising contacting Rho kinase with a compound selected from the group consisting of Examples 1 to 534.

3. A method of treatment of a Rho kinase-mediated disease, in a patient in need of such treatment, comprising the administration of a therapeutically effective amount of a compound of Formula I 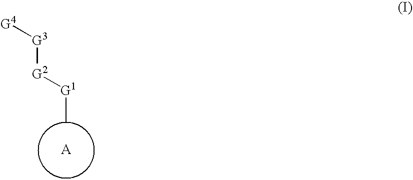
or a salt, ester, or prodrug thereof, wherein:
A is optionally substituted heteroaryl;
G¹ is optionally substituted 5-membered heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted.

4. The method as recited in claim 3 wherein said Rho kinase-mediated disease is selected from the group consisting of angina, coronary artery vasospasm, myocardial infarction, coronary ischemia, congestive heart failure, cardiac allograft vasculopathy, vein graft disease and vascular restenosis, ischemic reperfusion injury, transplant reperfusion injury, cerebral artery vasospasm, stroke, cerebral ischemia, essential hypertension, pulmonary hypertension, renal hypertension, a secondary hypertensive disorder, atherosclerosis, bronchial asthma, an acute or chronic obstructive pulmonary disease, an acute or chronic pulmonary inflammatory disease, erectile dysfunction, a neurodegenerative disorder, Alzheimer's disease, multiple sclerosis, brain or spinal cord injury, a disease or trauma-related neuropathy, neuropathic pain, an autoimmune disease, a chronic musculoskeletal inflammatory disease, rheumatoid arthritis, osteoarthritis, a chronic inflammatory bowel disease, Crohn's disease, ulcerative colitis, acute or chronic inflammatory pain, osteoporosis, a bone disorder, cancer, a disease of pathological angiogenesis, and an ophthalmic disease.

5. The method as recited in claim 4, wherein said Rho kinase-mediated disease is an ophthalmic disease.

6. The method as recited in claim 5, wherein said ophthalmic disease is selected from the group consisting of elevated intraocular pressure and glaucoma.

7. A method of treatment of a Rho kinase-mediated disease, in a patient in need of such treatment, comprising the administration of a therapeutically effective amount of a compound selected from the group consisting of Examples 1 to 534.

8. A method of treatment of a Rho kinase-mediated disease comprising the administration of
a. a therapeutically effective amount of structural Formula I 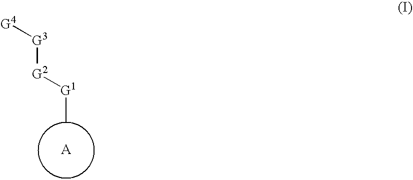
or a salt, ester, or prodrug thereof, wherein:
A is optionally substituted heteroaryl;
G¹ is optionally substituted 5-membered heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted; and
b. another therapeutic agent.

9. A method for
a. reducing apoptosis of human embryonic stem cells;
b. increasing survival of human embryonic stem cells;
c. increasing cloning efficiency of human embryonic stem cells after gene transfer; or
d. enhancing differentiation of cultured human embryonic stem cells, any one of said methods comprising the contacting of at least one human embryonic stem cell with an effective amount of structural Formula I 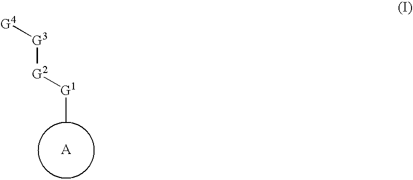
or a salt, ester, or prodrug thereof, wherein:
A is optionally substituted heteroaryl;
G¹ is optionally substituted 5-membered heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted.

10. A compound of either structural Formula II or structural Formula III 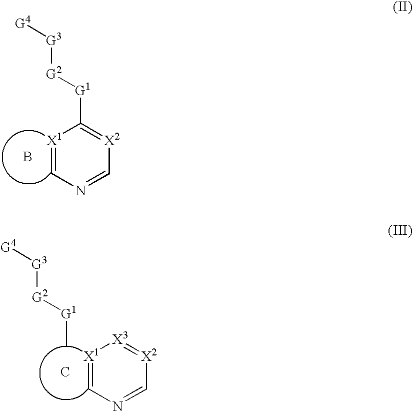
or a salt, ester, or prodrug thereof, wherein:
X¹ is C(R²) or N;
X² is C(R³) or N;
X³ is C(R⁴) or N;
B is selected from the group consisting of optionally substituted 5-membered heteroaryl and null;
C is optionally substituted 5-membered heteroaryl;
G¹ is optionally substituted 5-membered heteroaryl;
G² is selected from the group consisting of (CR^{a}R^{b})ₘZ(CR^{c}R^{d})ₚ and null;
m is 1, 2, 3, or 4;
p is 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted; and
R², R³, and R⁴ are each independently selected from the group consisting of acyl, lower alkenyl, alkynyl, lower alkoxy, lower alkoxyalkyl, lower alkyl, alkylthio, amido, amino, aminoalkyl, aminocarbonyl, aralkyl, arylcarbonyl, arylsulfonyl, cycloalkyl, cycloalkylalkyl, carboxyl, cycloalkenyl, halo, haloalkyl, hydroxyalkyl, heteroaryl, heteroaralkyl, heterocycloalkyl, heterocycloalkylalkyl and hydrogen, any of which may be optionally substituted.

11. A compound of either structural Formula IV or structural Formula V 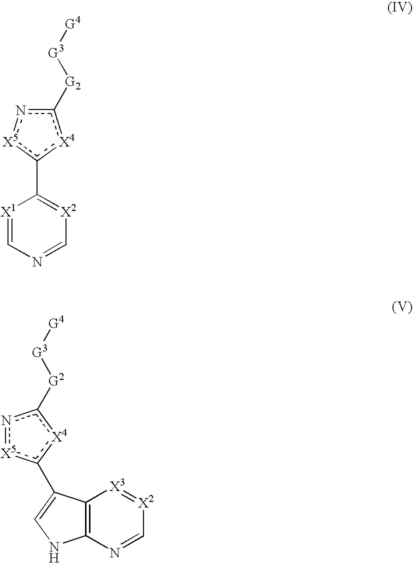
or a salt, ester, or prodrug thereof, wherein:
X¹ is C(R²) or N;
X² is C(R³) or N;
X³ is C(R⁴) or N;
X⁴ is selected from the group consisting of C(R⁵), O, N, and S;
X⁵ is selected from the group consisting of C(R⁶) and N;
G² is selected from the group consisting of (CR^{a}R^{b})ₘZ(CR^{c}R^{d})ₚ and null;
m is 1, 2, 3, or 4;
p is 0, 1, 2, 3, or 4;
Z is selected from the group consisting of O, N(R¹), S(O)ₙ, N(R^{e})CO, CON(R^{e}), N(R^{e})SO₂, SO₂N(R^{e}), carbonyl, optionally substituted cycloalkyl, and null;
R^{e} is selected from the group consisting of hydrogen and C₁-C₄ alkyl;
n is 0, 1 or 2;
R^{a}, R^{b}, R^{c}, and R^{d} are independently selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl;
G³ is selected from the group consisting of alkyl, alkylene, alkylamino, alkynyl, aryl, arylalkyl, arylalkoxy, carbonyl, carboxyl, cycloalkyl, heteroaryl, heteroarylalkyl, and heterocycloalkyl, any of which may be optionally substituted;
G⁴ is selected from the group consisting of hydrogen, hydroxy, C₁-C₄ alkyl, C₁-C₄ alkoxy, C₁-C₄ hydroxyalkyl, alkylcarboxyl, heterocycloalkylalkylamido, and aminoalkylcarboxyl, any of which may be optionally substituted; and
R¹ is selected from the group consisting of alkyl, alkylcarbonyl, alkylene, alkynyl, carbonyl, cycloalkyl, ester, heterocycloalkyl, heterocycloalkylalkyl and hydrogen, any of which may be optionally substituted;
R², R³, and R⁴ are each independently selected from the group consisting of acyl, lower alkenyl, alkynyl, lower alkoxy, lower alkoxyalkyl, lower alkyl, alkylthio, amido, amino, aminoalkyl, aminocarbonyl, aralkyl, arylcarbonyl, arylsulfonyl, cycloalkyl, cycloalkylalkyl, carboxyl, cycloalkenyl, halo, haloalkyl, hydroxyalkyl, heteroaryl, heteroaralkyl, heterocycloalkyl, heterocycloalkylalkyl and hydrogen, any of which may be optionally substituted; and
R⁵ and R⁶ are each independently selected from the group consisting of lower alkenyl, alkynyl, lower alkoxy, lower alkoxyalkyl, lower alkyl, alkylthio, amino, aminoalkyl, aminocarbonyl, arylcarbonyl, cycloalkyl, carboxy, haloalkyl, hydroxyalkyl, heterocycloalkyl and hydrogen, any of which may be optionally substituted.

12. The compound as recited in claim 11, or a salt, ester, or prodrug thereof, wherein:
G² is (CR^{a}R^{b})ₘZ(R^{c}R^{d})ₚ;
p is 0;
m is 1;
Z is N(R^{e})CO; and
R^{e} is hydrogen.

13. The compound as recited in claim 12, wherein:
R^{a} is selected from the group consisting of hydrogen, lower alkyl, amino, aminoalkyl, amidoalkyl, aminoalkylcarboxyl, carboxylalkyl, halo, heterocycloalkylalkyl, hydroxyalkyl, heteroarylalkyl and heterocycloalkylalkylcarboxyl; and
R^{b} is hydrogen.

14. The compound as recited in claim 13, wherein R^{a} is selected from the group consisting of hydrogen, lower alkyl, aminoalkyl, amidoalkyl, carboxylalkyl, heterocycloalkylalkyl, and heteroarylalkyl.

15. The compound as recited in claim 14, or a salt, ester, or prodrug thereof, wherein:
X⁴ is selected from the group consisting of C(R⁵) and N;
G³ is selected from the group consisting of aryl and heteroaryl, any of which may be optionally substituted; and
R⁵ and R⁶ are hydrogen.

16. The compound as recited in claim 15, or a salt, ester, or prodrug thereof, wherein:
X¹ is C(R²);
X² is C(R³);
X³ is C(R⁴); and
R², R³, and R⁴ are hydrogen.

17. The compound as recited in claim 16, or a salt, ester, or prodrug thereof, wherein G³ has a formula selected from the group consisting of: 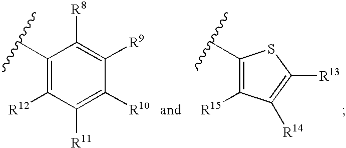
R⁸, R⁹, R¹¹, R¹², R¹³, R¹⁴, and R¹⁵ are independently selected from the group consisting of hydrogen, and at least one substituent selected from the group consisting of acyl, O-acyl, amino, amido, aryl, arylalkoxy, lower alkyl, lower alkoxy, carboxyl, cycloalkyl, hydroxy, halo, lower haloalkyl, lower haloalkoxy, heterocycloalkyl, heteroaryl, nitro, phenoxy and sulfonyl; and
R¹⁰ is selected from the group consisting of acyl, O-acyl, amino, amido, lower alkyl, C₂-C₆ alkoxy, carboxyl, cycloalkyl, fluorine, bromine, iodine, lower haloalkyl, lower haloalkoxy, heterocycloalkyl, heteroaryl, hydrogen, hydroxy, phenoxy, nitro and sulfonyl.

18. A compound selected from the group consisting of Examples 1 to 15, 17, 18, 20 to 79 and 82to 534.

19. A compound as recited in claim 10 for use as a medicament.

20. A compound as recited in claim 10 for use in the manufacture of a medicament for the prevention or treatment of a disease or condition ameliorated by the inhibition of Rho kinase.

21. A pharmaceutical composition comprising a compound as recited in claim 10 together with a pharmaceutically acceptable carrier.
